(19) United States
(12) Patent Application Publication
(10) Pub. No.: US 2025/0059217 A1
(43) Pub. Date: Feb. 20, 2025

(54) HETEROCYCLIC COMPOUNDS AND METHODS OF PREPARATION THEREOF

(71) Applicants: Bright Minds Biosciences Inc., Vancouver (CA); The Medical College of Wisconsin Inc., Milwaukee, WI (US)

(72) Inventors: Alan Kozikowski, Chicago, IL (US); Werner Tueckmantel, Fitchburg, WI (US); John McCorvy, Brookfield, WI (US); Uro Laban, Salvadore (BR)

(21) Appl. No.: 18/562,587

(22) PCT Filed: May 25, 2022
(86) PCT No.: PCT/CA2022/050833

§ 371 Date: Nov. 20, 2023

Related U.S. Application Data
(60) Provisional application No. 63/309,735, filed on Feb. 14, 2022.
(60) Provisional application No. 63/193,062, filed on May 26, 2021.

(51) Int. Cl. C07F 9/572; A61K 31/4045; A61K 31/5383; A61K 31/675; C07D 209/16; C07D 403/06; C07D 498/06

(52) U.S. Cl. CPC C07F 9/5728; A61K 31/4045; A61K 31/5383; A61K 31/675; C07D 209/16; C07D 403/06; C07D 498/06

(57) ABSTRACT

This disclosure relates to heterocyclic compounds of Formula (I), Formula (II), and Formula (III) as well as the preparation and use thereof. As contemplated herein, heterocyclic compounds of Formula (I), Formula (II), and Formula (III) may be used for the treatment of neuropsychiatric, and neurodegenerative, neuroinflammatory and pain disorders including depression, as well as tobacco, opiate, and cocaine addiction, alcoholism, post-traumatic stress disorder (PTSD), and neuropathic pain syndromes including cluster headaches and chemotherapy induced peripheral neuropathy. Formula (I), Formula (II), Formula (III).

### CROSS-REFERENCE

This application claims the benefit of: (i) U.S. Patent Application No. 63/193,062, filed May 26, 2021; and (ii) U.S. Patent Application No. 63/309,735, filed Feb. 14, 2022.

### TECHNICAL FIELD

This present disclosure relates to heterocyclic compounds and methods of preparing the same. This present disclosure also relates to uses of heterocyclic compounds as selective agents at serotonin receptors.

### BACKGROUND

Psilocybin is a naturally occurring psychedelic compound produced by more than 200 species of mushrooms collectively known as “psilocybin mushrooms”. As a prodrug, psilocybin is quickly metabolized by the body to generate the bioactive compound psilocin, which has mind-altering effects not unlike those produced by other psychedelics such as lysergic acid diethylamide (LSD), mescaline, and N,N-dimethyltryptamine (DMT). These effects include, inter alia, euphoria, visual and mental hallucinations, changes in perception, a distorted sense of time, and spiritual experiences, and can also include possible adverse reactions such as nausea and panic attacks. For reference, the chemical structure of psilocin is provided as follows:

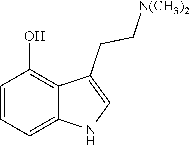 [SMILES: CN(C)CCc1c[nH]c2cccc(O)c12]

As agonists of the 5-HT_{2A} and 5-HT_{2C} receptors, psilocybin and psilocin have been recognized for their therapeutic potential. Since 5-HT_{2A} receptor activation appears to increase locomotor activity, whereas 5-HT_{2C} receptor activation appears to decrease locomotor activity, compounds possessing varying degrees of 5-HT_{2A} and 5-HT_{2C} activity will show varying levels of psychedelic activity (Halberstadt A L, van der Heijden I, Ruderman M A, Risbrough V B, Gingrich J A. Geyer M A, Powell S B, Neuropsychopharmacology, 2009, 34(8):1958-67). While psilocybin, along with other psychedelic drugs, were explored more than 60 years ago by Hofmann and co-workers at Sandoz (see for example, Hofmann, A., Troxler, F. U.S. Pat. Nos. 3,075,992; 3,078,214), clinical investigations into these drugs waned substantially by the early 1970s—particularly after these drugs were placed on Schedule 1 of the Controlled Substance Act in the United States of America. Notwithstanding their listing as controlled substances in certain jurisdictions however, research into psilocybin and other psychedelic drugs never fully stopped, and recent clinical investigations have led to a revived interest in the potential application of psychedelic drugs (including psilocybin) in evolving medical areas, such as the treatment of central nervous system (CNS) diseases. CNS diseases include both difficult-to-treat mental health disorders (Daniel J, Haberman M. Clinical potential of psilocybin as a treatment for mental health conditions. Ment. Health Clin. 2017, 7(1), 24-8), such as treatment resistant depression or drug resistant depression, and neurological disorders such as cluster headaches.

While psilocybin has recognized therapeutic potential for treating certain CNS diseases and disorders, it is also recognized as a 5-HT_{2B} receptor agonist and is therefore cardiotoxic. As such, there is an unmet need for safer drugs and analogs of psilocybin and psilocin that maintain 5-HT_{2A} receptor agonist activity but that lack cardiotoxic 5-HT_{2B} agonist activity; furthermore, and at least in some instances, there is an unmet need for safer drugs that maintain 5-HT_{2A} receptor agonist activity but that lack cardiotoxic 5-HT_{2B} agonist activity.

### SUMMARY

The present disclosure relates to indole compounds, such as 7-substituted indole compounds and 5-substituted indole compounds, that exhibit 5-HT_{2A} receptor agonist activity while exhibiting low 5HT_{2B} receptor agonist activity. In at least some cases, such compounds also show selectivity for the 5-HT_{2A} receptor over the 5-HT_{2C} receptor. The compounds disclosed herein may be useful in the treatment of depression including major depressive disorder, drug resistant depression, and psychotic depression, addiction including alcoholism, tobacco addiction, cocaine addiction, and opioid addiction, pain indications including neuropathic pain, pain from chemotherapy associated neuropathy, phantom limb pain and fibromyalgia, inflammation (including chronic and acute), eating disorders including anorexia, autism, cluster headaches, migraines, dementia including Alzheimer's dementia, Parkinson's disease dementia, and Lewy body dementia, post-traumatic stress disorder, emotional distress associated with cancer, Fragile-X syndrome, autism spectrum disorder, bipolar disease, obsessive compulsive disease, Rett syndrome, and other CNS disorders.

According to a part of the present disclosure, there are chemical entities of Formula I,

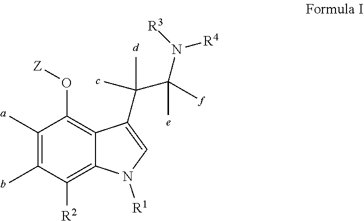

wherein R¹, R², R³, R⁴, a, b, c, d, e, f, and Z are defined hereinafter, and wherein R² is substituted.

The chemical entities of Formula I are 5-HT_{2A} receptor agonists with selectivity over the 5-HT_{2B} subtype. Chemical entities of Formula I, and pharmaceutically acceptable compositions thereof, are potentially useful for treating a variety of diseases and disorders associated with 5-HT2A receptor agonism. Such diseases and disorders include those described herein.

According to a part of the present disclosure, there are chemical entities of Formula II,

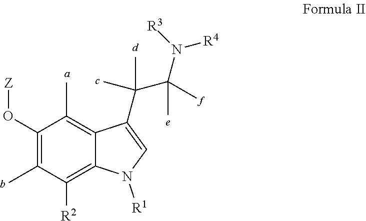

wherein R¹, R², R³, R⁴, a, b, c, d, e, f, and Z are defined hereinafter.

The chemical entities of Formula II are 5-HT_{2A} receptor agonists with selectivity over the 5-HT_{2B} subtype. Chemical entities of Formula II, and pharmaceutically acceptable compositions thereof, are potentially useful for treating a variety of diseases and disorders associated with 5-HT_{2A} receptor agonism. Such diseases and disorders include those described herein.

According to a part of the present disclosure, there are chemical entities of Formula III,

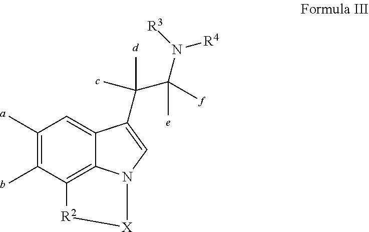

wherein R³, R⁴, a, b, c, d, e, and f are defined hereinafter, and wherein R² is O or S.

The chemical entities of Formula III are 5-HT_{2A} receptor agonists with selectivity over the 5-HT_{2B} subtype. Chemical entities of Formula III, and pharmaceutically acceptable compositions thereof, are potentially useful for treating a variety of diseases and disorders associated with 5-HT_{2A} receptor agonism. Such diseases and disorders include those described herein.

This summary does not necessarily describe the entire scope of all aspects of the disclosure. Other aspects, features and advantages will be apparent to those of ordinary skill in the art upon review of the following description of specific embodiments.

### DETAILED DESCRIPTION

Directional terms such as “top,” “bottom,” “upwards,” “downwards,” “vertically,” and “laterally” are used in the following description for the purpose of providing relative reference only, and are not intended to suggest any limitations on how any article is to be positioned during use, or to be mounted in an assembly or relative to an environment. The use of the word “a” or “an” when used herein in conjunction with the term “comprising” may mean “one,” but it is also consistent with the meaning of “one or more,” “at least one” and “one or more than one.” Any element expressed in the singular form also encompasses its plural form. Any element expressed in the plural form also encompasses its singular form. The term “plurality” as used herein means more than one, for example, two or more, three or more, four or more, and the like.

As used herein and unless otherwise specified, the term “about”, when used to describe a recited value, means within 10% of the recited value.

As used herein and unless otherwise specified, the term “alkenyl” refers to a substituted or unsubstituted, linear or branched, univalent hydrocarbon chain having at least two carbon atoms and at least one carbon-carbon (CC) double bond. Examples of alkenyl groups include allyl, vinyl, 2-propenyl, crotyl, 2-isopentenyl, 1,3-butadien-2-yl, 2,4-pentadien-1-yl, 1,4-pentadien-3-yl, and the like.

As used herein and unless otherwise specified, the term “alkoxy”, used alone or as part of a larger moiety, refers to the groups —O-alkyl and —O-cycloalkyl. As used herein and unless otherwise specified, the term “substituted alkoxy”, used alone or as part of a larger moiety, refers to the groups —O-(substituted alkyl) and —O-(substituted cycloalkyl).

As used herein and unless otherwise specified, the term “alkyl”, used alone or as part of a larger moiety, means a substituted or unsubstituted, linear or branched, univalent hydrocarbon chain that is completely saturated. Unless otherwise specified, an alkyl group contains 1 to 7 carbon atoms (“C₁-C₇ alkyl”). For example, in some embodiments, alkyl groups contain 1 to 6 carbon atoms (“C₁-C₆ alkyl”); in some embodiments, alkyl groups contain 1 to 5 carbon atoms (“C₁-C₅ alkyl”); in some embodiments, alkyl groups contain 1 to 4 carbon atoms (“C₁-C₄ alkyl”, alternatively “lower alkyl”); and in some embodiments, alkyl groups contain 3 to 7 carbon atoms (“C₃-C₇ alkyl”). Non-limiting examples of saturated alkyl groups include methyl, ethyl, n-propyl, i-propyl, n-butyl, t-butyl, i-butyl, s-butyl, homologs and isomers of, for example, n-pentyl, n-hexyl, n-heptyl, n-octyl, and the like. Examples of lower alkyl groups include methyl, ethyl, n-propyl, i-propyl, n-butyl, s-butyl, i-butyl, and t-butyl. A substituted alkyl group is one having at least one but no more than five substituents, and no more substituents than the number of hydrogen atoms in the unsubstituted group. In some embodiments, the substituents are fluorine atoms. Non-limiting examples of substituted alkyl groups include 2-hydroxyethyl, 2-methoxyethyl, CHF₂, CF₃, CH₂CF₃, CF₂CF₃, 4-fluorobutyl, and the like.

As used herein and unless otherwise specified, the term “alkynyl” refers to a substituted or unsubstituted, linear or branched, univalent hydrocarbon chain having at least two carbon atoms and at least one carbon-carbon triple bond. Non-limiting examples of alkynyl groups include ethynyl, 1- and 3-propynyl, 3-butyn-1-yl, and the like.

As used herein and unless otherwise specified, the term “aryl”, used alone or as part of a larger moiety (for example, “(aryl)alkyl”) refers to a univalent monocyclic or bicyclic carbocyclic aromatic ring system. Unless otherwise specified, aryl groups contain 6 or 10 ring members. Non-limiting examples of aryl include phenyl, naphthyl, and the like. The term “aryl” also refers to aryl groups that may be unsubstituted or substituted. For example, aryl groups can be unsubstituted or can be substituted with one, two, or three groups selected independently from the group consisting of halogen, OH, C₁-C₆ alkoxy, substituted C₁-C₆ alkoxy, C₁-C₆ alkylthio, substituted C₁-C₆ alkylthio, C₁-C₆ alkyl, substituted C₁-C₆ alkyl, C₃-C₆ cycloalkyl, substituted C₃-C₆ cycloalkyl, C(O)OH, C(O)(C₁-C₆ alkyl), C(N—OH)(C₁-C₆ alkyl), C(O)(C₁-C₆ alkoxy), C(O)NH₂, C(O)NH(C₁-C₆ alkyl), C(O)N(C₁-C₄ alkyl)(C₁-C₄ alkyl), C(O)-heterocyclyl, NHC(O)(C₁-C₆ alkyl), N(CH₃)C(O)(C₁-C₆ alkyl), and cyano.

As used herein and unless otherwise specified or clear from context, the term “chemical entity” refers to a compound having the indicated structure, whether in its “free” form (e.g., “free compound” or “free base” or “free acid” form, as applicable), or in a salt form, particularly a pharmaceutically acceptable salt form, and furthermore whether in solid state form or otherwise. In some embodiments, a solid state form is an amorphous (i.e., non-crystalline) form; in some embodiments, a solid state form is a crystalline form (e.g., a polymorph, pseudohydrate, hydrate, or solvate). Similarly, the term encompasses the compound whether provided in solid form or otherwise. Unless otherwise specified, all statements made herein regarding “compounds” apply to the associated chemical entities, as defined.

As used herein and unless otherwise specified, the terms “comprising”, “having”, “including”, “containing”, and grammatical variations thereof, are inclusive or open-ended and do not exclude additional, un-recited elements and/or method steps. For example, “A includes 1, 2, and 3” means that A includes but is not limited to 1, 2, and 3.

As used herein and unless otherwise specified, the term “consisting essentially of” when used herein in connection with a composition, use, or method, denotes that additional elements, method steps or both additional elements and method steps may be present, but that these additions do not materially affect the manner in which the recited composition, method, or use functions.

As used herein and unless otherwise specified, the term “consisting of” when used herein in connection with a composition, use, or method, excludes the presence of additional elements and/or method steps.

As used herein and unless otherwise specified, the term “cycloalkyl”, used alone or as part of a larger moiety, for example “(cycloalkyl)alkyl”, refers to: (i) a substituted or unsubstituted, univalent monocyclic hydrocarbon radical that is completely saturated or that contains one or more units of unsaturation, but which is not aromatic; or (ii) bicyclo[m.n.o]alkyl wherein each of “m”, “n”, and “o” is independently an integer ranging from zero to 5, and the sum “m”+“n”+“o” ranges from 2 to 6. In some embodiments, cycloalkyl groups contain 3 to 8 ring carbon atoms (“C₃-C₈ cycloalkyl”). Non-limiting examples of cycloalkyl include cyclopropyl, cyclobutyl, cyclopentyl, cyclohexyl, 1-cyclohexenyl, 3-cyclohexenyl, cycloheptyl, and the like, as well as bicyclo[2.2.1]heptyl (also called norbornyl) and bicyclo[1.1.1]pentyl. A substituted cycloalkyl group is one having at least one but no more than five substituents. In some embodiments, the substituents are fluorine atoms. Non-limiting examples of substituted cycloalkyl groups include 2-methylcyclopropyl, 4-hydroxycyclohexyl, 2-methoxycyclopentyl, 4,4-difluorocyclohexyl, and the like.

As used herein and unless otherwise specified, the term “halogen” or “halo”, used alone or as part of a larger moiety, refers to fluoro, chloro, bromo, or iodo.

As used herein and unless otherwise specified, the term “heteroalkyl” refers to a substituted or unsubstituted, saturated or unsaturated alkyl group, as defined herein, in which one or more of the constituent carbon atoms have been replaced by nitrogen, oxygen, or sulfur.

As used herein and unless otherwise specified, the term “heteroaryl”, used alone or as part of a larger moiety, e.g., “(heteroaryl)alkyl”, refers to a univalent monocyclic or bicyclic group having 5 to 10 ring atoms, preferably 5, 6, 9, or 10 ring atoms, having 6 or 10 π electrons shared in a cyclic array, and having, in addition to ring carbon atoms, from one to four ring heteroatoms. Examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolyl, indolizinyl, benzofuranyl, benzothiophenyl, benzimidazolyl, benzoxazolyl, benzothiazolyl, benzotriazolyl, quinolyl, isoquinolyl, purinyl, naphthyridinyl, pteridinyl, and the like. Heteroaryl groups may be unsubstituted or may be substituted with one, two, or three groups selected independently from halogen, OH, C₁-C₆ alkoxy, substituted C₁-C₆ alkoxy, C₁-C₆ alkylthio, substituted C₁-C₆ alkylthio, C₁-C₆ alkyl, substituted C₁-C₆ alkyl, C₃-C₆ cycloalkyl, substituted C₃-C₆ cycloalkyl, C(O)OH, C(O)(C₁-C₆ alkoxy), C(O)NH₂, C(O)NH(C₁-C₆ alkyl), C(O)N(C₁-C₄ alkyl)(C₁-C₄ alkyl), C(O)-heterocyclyl, NHC(O)(C₁-C₆ alkyl), N(CH₃)C(O)(C₁-C₆ alkyl), and cyano.

As used herein and unless otherwise specified, the term “heterocyclyl”, used alone or as part of a larger moiety (for example, “(heterocyclyl)alkyl”) refers to a univalent stable 4- to 7-membered monocyclic or 7- to 10-membered bicyclic heterocyclic moiety that is either saturated or partially unsaturated, and has, in addition to ring carbon atoms, one to four heteroatoms. Non-limiting examples of heterocyclyl groups include tetrahydrofuranyl, pyrrolidinyl, tetrahydropyranyl, piperidinyl, morpholinyl, and the like. Heterocyclyl groups can be unsubstituted or can be substituted. For example, heterocyclyl groups can be unsubstituted or can be substituted with one, two, or three groups selected independently from the group consisting of halogen, OH, O(C₁-C₆ alkyl), O(substituted C₁-C₆ alkyl), C₁-C₆ alkyl, substituted C₁-C₆ alkyl, and C₃-C₆ cycloalkyl.

As used herein and unless otherwise specified, the term “inactive” (and all related terms thereto including “inactivity”), when used the context of “EC₅₀ (nM)” and “Eff %” as such terms would be understood by a person skilled in the art or equivalent skilled person, and when used in reference to the activity at the 5-HT_{2B} receptor, means a concentration of greater than 10,000 nM (when used in the context of “EC₅₀ (nM)”) or an efficacy of 30% or lower (when used in the context of “Eff %”).

As used herein and unless otherwise specified, the term “isotopologue” refers to a species that differs from a specific compound only in the isotopic composition thereof. For example, all hydrogen atoms in a compound are independently of natural isotopic composition or of any isotopic composition enriched or depleted in one or both of the heavy isotopes, ²H (D, deuterium) and ³H (T, tritium), ranging from a depletion to zero % to an enrichment to 100%.

As used herein and unless otherwise specified, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts of the compounds provided in this disclosure include salts derived from suitable inorganic and organic acids and bases. Non-limiting examples of pharmaceutically acceptable salts include salts of compounds comprising an amino group that are formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid. Other non-limiting examples of pharmaceutically acceptable salts include adipate, alginate, ascorbate, aspartate, benzenesulfonate, benzoate, bisulfate, borate, butyrate, camphorate, camphorsulfonate, cyclopentanepropionate, digluconate, dodecylsulfate, ethanesulfonate, formate, fumarate, glucoheptonate, glycerophosphate, gluconate, hemisulfate, heptanoate, hexanoate, hydriodide, 2-hydroxyethanesulfonate, lactobionate, lactate, laurate, lauryl sulfate, malate, methanesulfonate, 2-naphthalenesulfonate, nicotinate, nitrate, oleate, palmitate, pamoate, pectinate, persulfate, 3-phenylpropionate, pivalate, propionate, stearate, thiocyanate, p-toluenesulfonate, undecanoate, valerate salts, and the like. Other pharmaceutically acceptable salts include those that are derived from appropriate bases such as alkali metal, alkaline earth metal, ammonium, and N⁺(C₁₄ alkyl)₄ salts. Representative alkali or alkaline earth metal salts include sodium, lithium, potassium, calcium, magnesium, and the like. Further non-limiting examples of pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein and unless otherwise specified, the term “subject” includes a mammal (e.g., a human, and in some embodiments including prenatal human forms). In some embodiments, a subject suffers from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is a mammal with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, a subject is a fetus, an infant, a child, a teenager, an adult, or a senior citizen (i.e., the subject is of advanced age, such as older than 50). In some embodiments, a child refers to a human that is between two and 18 years of age. In some embodiments, an adult refers to a human that is eighteen years of age or older.

As used herein and unless otherwise specified, the phrase “such as” is intended to be open-ended. For example, the phrase “A can be a halogen, such as chlorine or bromine” means that “A” can be, but is not limited to, chlorine or bromine.

Reference to specific moieties, functional groups, or substituents contemplates (where applicable) tautomers thereof.

Unless otherwise stated, structures depicted herein include all isomeric (e.g., enantiomeric, diastereomeric, and geometric (or conformational)) forms of the structure (e.g., the R and S configurations for each asymmetric center, Z and E double bond isomers, and Z and E conformational isomers). Unless otherwise stated, the compounds disclosed, taught, or otherwise suggested in this disclosure contemplate all single stereochemical isomers as well as enantiomeric, diastereomeric, and geometric (or conformational) mixtures thereof. Unless otherwise stated, the compounds disclosed, taught, or suggested in this disclosure contemplate all tautomeric forms thereof. Additionally, unless otherwise stated, structures depicted herein include compounds that differ only in the presence of one or more isotopically enriched atoms. Such compounds may be useful, for example, as analytical tools, as probes in biological assays, or as therapeutic agents. Additionally, incorporation of heavier isotopes such as deuterium (²H) may afford certain therapeutic advantages resulting from greater metabolic stability, for example, increase in vivo half-life, or reduced dosage requirements.

Chemical entities described herein are further illustrated by the classes, subclasses, and species disclosed herein. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. In this disclosure, any atom not specifically designated as a particular isotope is meant to represent any stable isotope of that atom.

Unless otherwise stated, structures depicted herein are also meant to include all stereoisomeric (e.g., enantiomeric, diastereomeric, and geometric (or conformational)) forms of the structure; for example, the R and S configurations for each asymmetric center, Z and E double bond isomers, and Z and E conformational isomers. Therefore, the present compounds contemplate all single stereochemical isomers as well as enantiomeric, diastereomeric, and geometric (or conformational) mixtures thereof. Unless otherwise stated, the present compounds contemplate all tautomeric forms thereof. Additionally, unless otherwise stated, structures depicted herein are also meant to include compounds that differ only in the presence of one or more isotopically enriched atoms. Such compounds may be useful, for example, as analytical tools, as probes in biological assays, or as therapeutic agents. Additionally, incorporation of heavier isotopes such as deuterium (²H) may afford certain therapeutic advantages resulting from greater metabolic stability, for example, increase in vivo half-life, or reduced dosage requirements.

According to some embodiments of the chemical entities disclosed herein, there are chemical entities of Formula I:

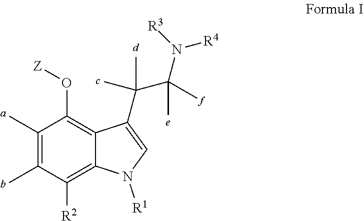

wherein:
R¹: (i) is selected from the group consisting of H, C₁-C₆ alkyl, C₁-C₆ substituted alkyl, C₂-C₆ alkenyl, C₂-C₆ alkynyl, C₃-C₆ cycloalkyl, (C₃-C₆ cycloalkyl)(C₁-C₆ alkyl), C₃-C₆ heterocyclyl, (C₃-C₆ heterocyclyl)(C₁-C₆ alkyl), aryl(C₁-C₆ alkyl), and heteroaryl(C₁-C₆ alkyl); or (ii) together with R² form a chain of 2 to 4 carbon atoms to which are attached substituents independently selected from the group consisting of H, C₁-C₆ alkyl, aryl, heteroaryl, and any combination thereof;
R²: (i) is selected from the group consisting of C₁-C₆ alkyl, C₁-C₆ substituted alkyl, C₂-C₆ alkenyl, C₂-C₆ alkynyl, C₃-C₆ cycloalkyl, (C₃-C₆ cycloalkyl)(C₁-C₆ alkyl), C₃-C₆ heterocyclyl, (C₃-C₆ heterocyclyl)(C₁-C₆ alkyl), aryl, aryl(C₁-C₆ alkyl), heteroaryl, heteroaryl(C₁-C₆ alkyl), CN, C(O)NH₂, C(O)NH(C₁-C₆ alkyl), C(O)N(C₁-C₃ alkyl)(C₁-C₆ alkyl), C(═NOH)(C₁-C₆ alkyl), and C(═NOH)(C₁-C₆ substituted alkyl); or (ii) together with R¹ form a chain of 2 to 4 carbon atoms to which are attached substituents independently selected from the group consisting of H, C₁-C₆ alkyl, aryl, and heteroaryl; or (iii) together with b form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCF₃, cyano, and oxo; or (iv) is selected from the group consisting of H, C₁-C₆ alkyl, C₁-C₆ substituted alkyl, C₂-C₆ alkenyl, C₂-C₆ alkynyl, C₃-C₆ cycloalkyl, (C₃-C₆ cycloalkyl)(C₁-C₆ alkyl), C₃-C₆ heterocyclyl, (C₃-C₆ heterocyclyl)(C₁-C₆ alkyl), aryl, aryl(C₁-C₆ alkyl), heteroaryl, heteroaryl(C₁-C₆ alkyl), CN, C(O)NH₂, C(O)NH(C₁-C₆ alkyl), C(O)N(C₁-C₃ alkyl)(C₁-C₆ alkyl), C(═NOH)(C₁-C₆ alkyl), and C(═NOH)(C₁-C₆ substituted alkyl), if b is halogen, CH₃, CHF₂, CF₃, OCH₃, OCHF₂, OCF₃, SCH₃, SCHF₂, SCF₃, or cyano. In some embodiments, R² together with b form any one of CH₂CH₂, CH₂CH₂CH₂, CH₂CH₂CH₂CH₂, CH═CHCH═CH, OCH₂CH₂, CH₂OCH₂, CH₂CH₂O, OCH═CH, CH═CHO, OCH₂O, SCH₂CH₂, CH₂SCH₂, CH₂CH₂S, SCH═CH, CH═CHS, NHCH₂CH₂, CH₂NHCH₂, CH₂CH₂NH, NHCH═CH, CH═CHNH, ON═CH, CH═NO, OCH═N, N═CHO, SN═CH, CH═NS, SCH═N, N═CHS, NHN═CH, CH═NNH, NHCH═N, N═CHNH, NHN═N, N═NNH, OCH₂CH₂CH₂, CH₂OCH₂CH₂, CH₂CH₂OCH₂, CH₂CH₂CH₂O, SCH₂CH₂CH₂, CH₂SCH₂CH₂, CH₂CH₂SCH₂, CH₂CH₂CH₂S NHCH₂CH₂CH₂, CH₂NHCH₂CH₂, CH₂CH₂NCH₂, CH₂CH₂CH₂NH, N═CHCH═CH, CH═NCH═CH, CH═CHN═CH, CH═CHCH═N. In some embodiments, R² together with b form any one of CH₂CH₂, CH₂CH₂CH₂, CH₂CH₂CH₂CH₂, CH═CHCH═CH, OCH₂CH₂, CH₂OCH₂, CH₂CH₂O, OCH═CH, CH═CHO, OCH₂O, SCH₂CH₂, CH₂SCH₂, CH₂CH₂S, SCH═CH, CH═CHS, NHCH₂CH₂, CH₂NHCH₂, CH₂CH₂NH, NHCH═CH, CH═CHNH, ON═CH, CH═NO, OCH═N, N═CHO, SN═CH, CH═NS, SCH═N, N═CHS, NHN═CH, CH═NNH, NHCH═N, N═CHNH, NHN═N, N═NNH, OCH₂CH₂CH₂, CH₂OCH₂CH₂, CH₂CH₂OCH₂, CH₂CH₂CH₂O, SCH₂CH₂CH₂, CH₂SCH₂CH₂, CH₂CH₂SCH₂, CH₂CH₂CH₂S NHCH₂CH₂CH₂, CH₂NHCH₂CH₂, CH₂CH₂NCH₂, CH₂CH₂CH₂NH, N═CHCH═CH, CH═NCH═CH, CH═CHN═CH, CH═CHCH═N, wherein one hydrogen atom or two hydrogen atoms, if present on a moiety, are replaced with substituents selected independently from the group consisting of halogen, OH, C₁-C₆ alkoxy, C₁-C₆ alkyl, C₃-C₆ cycloalkyl, CHF₂, CF₃, OCHF₂, OCF₃, SCH₃, SCF₃, and cyano, or wherein two hydrogens, if attached to the same carbon atom, are replaced with an oxo group;
a: (i) is selected from the group consisting of H, halogen, lower alkyl, CHF₂, CF₃, OCH₃, OCHF₂, OCF₃, SCHF₂, SCH₃, SCF₃, and cyano; or (ii) together with Z form one of (A) a saturated chain of one oxygen and one carbon atom (with oxygen connected to the 5-position of the indole ring of Formula I), and (B) a chain of 2 or 3 carbon atoms, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo, and (C) a chain of 2 or 3 carbon atoms containing one double bond, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCHF₂, SCH₃, SCF₃, cyano, and oxo; or (iii) together with b form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo; and
b: (i) is selected from a group consisting of H, halogen, CH₃, CHF₂, CF₃, OCH₃, OCHF₂, OCF₃, SCH₃, SCHF₂, SCF₃, and cyano; or (ii) together with a form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo; or (iii) together with R² form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo;
   wherein:
R³: (i) is selected from the group consisting of H, C₁-C₆ alkyl, C₂-C₆ alkenyl, C₂-C₆ alkynyl, C₃-C₆ cycloalkyl, (C₃-C₆ cycloalkyl)(C₁-C₆ alkyl), aryl(C₁-C₆ alkyl), acetyl, and heteroaryl(C₁-C₆ alkyl); or (ii) together with R⁴ and the N atom to which they are attached form a 4-7 membered heterocyclyl ring; or (iii) together with f and the N atom to which R³ is attached form an azetidine or pyrrolidine ring, such ring carrying substituents independently selected from the group consisting of H, aryl, heteroaryl, C₁-C₆ alkyl, and C₃-C₆ cycloalkyl; or (iv) together with c and the N atom to which R³ is attached form an azetidine or pyrrolidine ring, such ring carrying substituents independently selected from the group consisting of H, aryl, heteroaryl, halogen, C₁-C₆ alkyl, and C₃-C₆ cycloalkyl;
R⁴: (i) is selected from the group consisting of H, C₁-C₆ alkyl, C₂-C₆ alkenyl, C₂-C₆ alkynyl, C₃-C₆ cycloalkyl, (C₃-C₆ cycloalkyl)(C₁-C₆ alkyl), aryl(C₁-C₆ alkyl), acetyl, and heteroaryl(C₁-C₆ alkyl); or (ii) together with R³ and the N atom to which they are attached form a 4-7 membered heterocyclyl ring;
   wherein:
c, d, e, and f are each H; or three of c, d, e, and f are H and the remaining substituent is a lower alkyl group; or c and f are each H, and d and e together are —CH₂— or —CH₂CH₂—, thereby giving rise to a cyclopropane or cyclobutane ring; or c, d, and e are each H, and f, R³, and the N atom to which R³ is attached form together an azetidine or pyrrolidine ring, such ring carrying substituents independently selected from the group consisting of H, aryl, heteroaryl, C₁-C₆ alkyl, and C₃-C₆ cycloalkyl; or d, e, and f are each H, and c, R³, and the N atom to which R³ is attached form together an azetidine or pyrrolidine ring, such ring carrying substituents independently selected from the group consisting of H, aryl, heteroaryl, halogen, C₁-C₆ alkyl, and C₃-C₆ cycloalkyl; or d, e, and f are each H, and c and Z together comprise 1 or 2 carbon atoms so as to give rise to a pyran or oxepan ring, such ring carrying substituents independently selected from the group consisting of H, halogen, C₁-C₆ alkyl, and C₃-C₆ cycloalkyl; and
   wherein:
Z: (i) is selected from the group consisting of H, R⁵, (R⁶)(R⁷)N—C(O)—, C₁-C₆ alkyl-C(O), C₃-C₆ cycloalkyl-C(O), aryl-C(O), and heteroaryl-C(O), wherein R⁵ is selected from the group consisting of C₁-C₆ alkyl, C₂-C₆ alkenyl, C₂-C₆ alkynyl, C₃-C₆ cycloalkyl, (C₃-C₆ cycloalkyl)(C₁-C₆ alkyl), aryl(C₁-C₆ alkyl), and heteroaryl(C₁-C₆ alkyl), and wherein R⁶ and R⁷ are each independently selected from the group consisting of H, C₁-C₄ alkyl, and C₃—C₆ cycloalkyl or are joined to form a 4-7 membered heterocyclyl group; or (ii) is (R⁸O)(R⁹O)P(O)—, wherein R⁸ and R⁹ are each independently H or a cationic counterion of a phosphate salt form such as sodium, potassium, one-half of magnesium, one-half of calcium, ammonium, or ammonium substituted with one or more alkyl or cycloalkyl groups; or (iii) together with c form a linkage that gives rise to a pyran or oxepan ring comprising substituents independently selected from the group consisting of H, halogen, C₁-C₆ alkyl, and C₃-C₆ cycloalkyl; or (iv) together with a form one of (A) a saturated chain of one oxygen and one carbon atom (with oxygen connected to the 5-position of the indole ring of Formula I), and (B) a chain of 2 or 3 carbon atoms, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo, and (C) a chain of 2 or 3 carbon atoms containing one double bond and carrying substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo.

According to other embodiments of the chemical entities disclosed herein, there are chemical entities of Formula II:

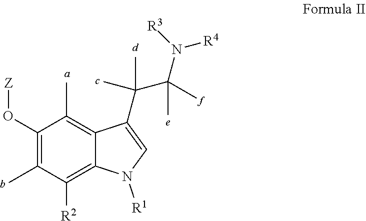

wherein:
R¹: (i) is selected from the group consisting of H, C₁-C₆ alkyl, C₁-C₆ substituted alkyl, C₂-C₆ alkenyl, C₂-C₆ alkynyl, C₃-C₆ cycloalkyl, (C₃-C₆ cycloalkyl)(C₁-C₆ alkyl), C₃-C₆ heterocyclyl, (C₃-C₆ heterocyclyl)(C₁-C₆ alkyl), aryl(C₁-C₆ alkyl), or heteroaryl(C₁-C₆ alkyl); or (ii) together with R² form a chain of 2 to 4 carbon atoms to which are attached substituents independently selected from the group consisting of H, C₁-C₆ alkyl, aryl, and heteroaryl;
R²: (i) is selected from the group consisting of C₁-C₆ alkyl, C₁-C₆ substituted alkyl, C₂-C₆ alkenyl, C₂-C₆ alkynyl, C₃-C₆ cycloalkyl, (C₃-C₆ cycloalkyl)(C₁-C₆ alkyl), C₃-C₆ heterocyclyl, (C₃-C₆ heterocyclyl)(C₁-C₆ alkyl), aryl, aryl(C₁-C₆ alkyl), heteroaryl, heteroaryl(C₁-C₆ alkyl), cyano, C(O)NH₂, C(O)NH(C₁-C₆ alkyl), C(═NOH)(C₁-C₆ alkyl), and C(═NOH)(C₁-C₆ substituted alkyl); or (ii) together with R² form a C₂-C₄ alkyl linkage comprising substituents independently selected from the group consisting of H, C₁-C₆ alkyl, aryl, and heteroaryl; or (iii) together with b form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo; or (iv) is H, if b is not H. In some embodiments, R² together with b form any one of CH₂CH₂, CH₂CH₂CH₂, CH₂CH₂CH₂CH₂, CH═CHCH═CH, OCH₂CH₂, CH₂OCH₂, CH₂CH₂O, OCH═CH, CH═CHO, OCH₂O, SCH₂CH₂, CH₂SCH₂, CH₂CH₂S, SCH═CH, CH═CHS, NHCH₂CH₂, CH₂NHCH₂, CH₂CH₂NH, NHCH═CH, CH═CHNH, ON═CH, CH═NO, OCH═N, N═CHO, SN═CH, CH═NS, SCH═N, N═CHS, NHN═CH, CH═NNH, NHCH═N, N═CHNH, NHN═N, N═NNH, OCH₂CH₂CH₂, CH₂OCH₂CH₂, CH₂CH₂OCH₂, CH₂CH₂CH₂O, SCH₂CH₂CH₂, CH₂SCH₂CH₂, CH₂CH₂SCH₂, CH₂CH₂CH₂S NHCH₂CH₂CH₂, CH₂NHCH₂CH₂, CH₂CH₂NCH₂, CH₂CH₂CH₂NH, N═CHCH═CH, CH═NCH═CH, CH═CHN═CH, CH═CHCH═N. In some embodiments, R² together with b form any one of CH₂CH₂, CH₂CH₂CH₂, CH₂CH₂CH₂CH₂, CH═CHCH═CH, OCH₂CH₂, CH₂OCH₂, CH₂CH₂O, OCH═CH, CH═CHO, OCH₂O, SCH₂CH₂, CH₂SCH₂, CH₂CH₂S, SCH═CH, CH═CHS, NHCH₂CH₂, CH₂NHCH₂, CH₂CH₂NH, NHCH═CH, CH═CHNH, ON═CH, CH═NO, OCH═N, N═CHO, SN═CH, CH═NS, SCH═N, N═CHS, NHN═CH, CH═NNH, NHCH═N, N═CHNH, NHN═N, N═NNH, OCH₂CH₂CH₂, CH₂OCH₂CH₂, CH₂CH₂OCH₂, CH₂CH₂CH₂O, SCH₂CH₂CH₂, CH₂SCH₂CH₂, CH₂CH₂SCH₂, CH₂CH₂CH₂S NHCH₂CH₂CH₂, CH₂NHCH₂CH₂, CH₂CH₂NHCH₂, CH₂CH₂CH₂NH, N═CHCH═CH, CH═NCH═CH, CH═CHN═CH, CH═CHCH═N, wherein one hydrogen atom or two hydrogen atoms, if present on a moiety, are replaced with substituents selected independently from the group consisting of halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, and cyano, and wherein two hydrogens, if attached to the same carbon atom, are replaced with an oxo group;
a: (i) is selected from the group consisting of H, halogen, CH₃, CHF₂, CF₃, OCH₃, OCHF₂, OCF₃, SCH₃, SCF₃, and cyano; or (ii) together with Z form one of (A) a saturated chain of 2 or 3 carbon atoms, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo, and (B) a chain of 2 or 3 carbon atoms containing one double bond, such chain carrying substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCF₃, cyano, and oxo; or (iii) together with b form any one of a saturated alkyl linkage, an unsaturated alkyl linkage, a saturated heteroalkyl linkage, and an unsaturated heteroalkyl linkage, comprising substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCF₃, cyano, and oxo; or (iv) together with c form one of (A) a saturated chain of one or two carbon atoms and one oxygen atom, and (B) a saturated chain of 2 or 3 carbon atoms, to which chain are attached substituents independently selected from the group consisting of H, halogen, C₁-C₆ alkyl, and C₃-C₆ cycloalkyl;
b: (i) is selected from the group consisting of H, halogen, CH₃, CHF₂, CF₃, OCH₃, OCHF₂, OCF₃, SCH₃, SCHF₂, SCF₃, and cyano; or (ii) together with R² form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo; or (iii) together with Z form one of (A) a saturated chain of one oxygen and one carbon atom (with oxygen connected to the 6-position of the indole ring of Formula II), and (B) a chain of 2 or 3 carbon atoms, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, oxo, and cyano, and (C) a chain of 2 or 3 carbon atoms containing one double bond and carrying substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, oxo, and cyano. In some embodiments, b together with R² form any one of CH₂CH₂, CH₂CH₂CH₂, CH₂CH₂CH₂CH₂, CH═CHCH═CH, OCH₂CH₂, CH₂OCH₂, CH₂CH₂O, OCH═CH, CH═CHO, OCH₂O, SCH₂CH₂, CH₂SCH₂, CH₂CH₂S, SCH═CH, CH═CHS, NHCH₂CH₂, CH₂NHCH₂, CH₂CH₂NH, NHCH═CH, CH═CHNH, ON═CH, CH═NO, OCH═N, N═CHO, SN═CH, CH═NS, SCH═N, N═CHS, NHN═CH, CH═NNH, NHCH═N, N═CHNH, NHN═N, N═NNH, OCH₂CH₂CH₂, CH₂OCH₂CH₂, CH₂CH₂OCH₂, CH₂CH₂CH₂O, SCH₂CH₂CH₂, CH₂SCH₂CH₂, CH₂CH₂SCH₂, CH₂CH₂CH₂S NHCH₂CH₂CH₂, CH₂NHCH₂CH₂, CH₂CH₂NCH₂, CH₂CH₂CH₂NH, N═CHCH═CH, CH═NCH═CH, CH═CHN═CH, and CH═CHCH═N. In some embodiments, b together with R² form any one of CH₂CH₂, CH₂CH₂CH₂, CH₂CH₂CH₂CH₂, CH═CHCH═CH, OCH₂CH₂, CH₂OCH₂, CH₂CH₂O, OCH═CH, CH═CHO, OCH₂O, SCH₂CH₂, CH₂SCH₂, CH₂CH₂S, SCH═CH, CH═CHS, NHCH₂CH₂, CH₂NHCH₂, CH₂CH₂NH, NHCH═CH, CH═CHNH, ON═CH, CH═NO, OCH═N, N═CHO, SN═CH, CH═NS, SCH═N, N═CHS, NHN═CH, CH═NNH, NHCH═N, N═CHNH, NHN═N, N═NNH, OCH₂CH₂CH₂, CH₂OCH₂CH₂, CH₂CH₂OCH₂, CH₂CH₂CH₂O, SCH₂CH₂CH₂, CH₂SCH₂CH₂, CH₂CH₂SCH₂, CH₂CH₂CH₂S NHCH₂CH₂CH₂, CH₂NHCH₂CH₂, CH₂CH₂NCH₂, CH₂CH₂CH₂NH, N═CHCH═CH, CH═NCH═CH, CH═CHN═CH, and CH═CHCH═N, wherein one hydrogen atom or two hydrogen atoms, if present on a moiety, are replaced with substituents selected independently from the group consisting of halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, and cyano, and wherein two hydrogens, if attached to the same carbon atom, are replaced with an oxo group;
   wherein:
R³: (i) is selected from the group consisting of H, C₁-C₆ alkyl, C₂-C₆ alkenyl, C₂-C₆ alkynyl, C₃-C₆ cycloalkyl, (C₃-C₆ cycloalkyl)(C₁-C₆ alkyl), aryl(C₁-C₆ alkyl), and heteroaryl(C₁-C₆ alkyl); or (ii) together with R⁴ and the N atom to which they are attached form a 4-7 membered heterocyclyl group; or (iii) together with f and the N atom to which R³ is attached form an azetidine or pyrrolidine ring, such ring comprising substituents independently selected from the group consisting of H, aryl, heteroaryl, C₁-C₆ alkyl, and C₃-C₆ cycloalkyl; or (iv) together with c and the N atom to which R³ is attached form an azetidine or pyrrolidine ring, such ring carrying substituents independently selected from the group consisting of H, aryl, heteroaryl, halogen, C₁-C₆ alkyl, and C₃-C₆ cycloalkyl;
R⁴: (i) is selected from the group consisting of H, C₁-C₆ alkyl, C₂-C₆ alkenyl, C₂-C₆ alkynyl, C₃-C₆ cycloalkyl, (C₃-C₆ cycloalkyl)(C₁-C₆ alkyl), aryl(C₁-C₆ alkyl), and heteroaryl(C₁-C₆ alkyl); or (ii) together with R³ and the N atom to which they are attached form a 4-7 membered heterocyclyl group;
   wherein:
c, d, e, and f are each H; or three of c, d, e, and f are each H, and the remaining substituent is a lower alkyl group; or c and f are H, and d and e together are —CH₂— or —CH₂CH₂—, thereby giving rise to a cyclopropane or cyclobutane ring; or c, d, and e are each H, and f, R³, and the N atom to which R³ is attached form together an azetidine or pyrrolidine ring, such ring carrying substituents independently selected from the group consisting of H, aryl, heteroaryl, C₁-C₆ alkyl, and C₃-C₆ cycloalkyl; or d, e, and f are each H, and c, R³, and the N atom to which R³ is attached form together an azetidine or pyrrolidine ring, such ring carrying substituents independently selected from the group consisting of H, aryl, heteroaryl, halogen, C₁-C₆ alkyl, and C₃-C₆ cycloalkyl; or d, e, and f are each H, and c and a together form a saturated chain of one or two carbon atoms and one oxygen atom, or a saturated chain of 2 or 3 carbon atoms, to which chain are attached substituents independently selected from the group consisting of H, aryl, heteroaryl, halogen, C₁-C₆ alkyl, and C₃-C₆ cycloalkyl;
Z: (i) is selected from the group consisting of H, R⁵, (R⁶)(R⁷)N—C(O)—, C₁-C₆ alkyl-C(O), C₃-C₆ cycloalkyl-C(O), aryl-C(O), or heteroaryl-C(O), wherein R⁵ is selected from the group consisting of C₁-C₆ alkyl, C₂-C₆ alkenyl, C₂-C₆ alkynyl, C₃-C₆ cycloalkyl, (C₃-C₆ cycloalkyl)(C₁-C₆ alkyl), aryl, aryl(C₁-C₆ alkyl), heteroaryl, and heteroaryl(C₁-C₆ alkyl), and wherein R⁶ and R⁷ are independently selected from the group consisting of H, C₁-C₄ alkyl, and C₃-C₆ cycloalkyl, or R⁶ and R⁷ may be joined to form a 4-7 membered heterocyclyl group; or (ii) is (R⁸O)(R⁹O)P(O)—, wherein R⁸ and R⁹ are independently H or a cationic counterion of a phosphate salt form such as sodium, potassium, one-half of magnesium, one-half of calcium, ammonium, or ammonium substituted with one or more alkyl or cycloalkyl groups; or (iii) together with a form one of (A) a saturated chain of 2 or 3 carbon atoms, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo, and (B) a chain of 2 or 3 carbon atoms containing one double bond and carrying substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo; or (iv) together with b form one of (A) a saturated chain of one oxygen and one carbon atom (with oxygen connected to the 6-position of the indole ring of Formula II), and (B) a chain of 2 or 3 carbon atoms, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, oxo, and cyano, and (C) a chain of 2 or 3 carbon atoms containing one double bond and carrying substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, oxo, and cyano; and
   wherein: (i) if Z is methyl, then R² is not methyl; and (ii) if R² is methyl, then Z is not methyl.

For certainty, the chemical entities of Formula II do not include 5-methoxy-7,N,N-trimethyltryptamine of natural hydrogen isotope composition, as such specific compound was disclosed in Glennon et al., J. Med. Chem., 1980, 23(11), 1222.

According to some embodiments of the chemical entities disclosed herein, there are chemical entities of Formula III:

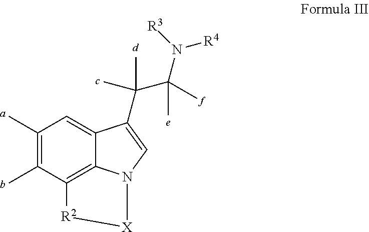

wherein:
R² is O or S;
X is a carbon chain that bonds together R² and the 1^{st} position of the indole ring structure and that contains 2 to 4 carbon atoms, to which carbon chain are attached substituents independently selected from the group consisting of H, C₁-C₆ alkyl, aryl, and heteroaryl;
a: (i) is selected from the group consisting of H, halogen, lower alkyl, CHF₂, CF₃, OCH₃, OCHF₂, OCF₃, SCHF₂, SCH₃, SCF₃, and cyano; or (ii) together with Z form one of (A) a saturated chain of one oxygen and one carbon atom (with oxygen connected to the 5-position of the indole ring of Formula I), and (B) a chain of 2 or 3 carbon atoms, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo, and (C) a chain of 2 or 3 carbon atoms containing one double bond, to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCHF₂, SCH₃, SCF₃, cyano, and oxo; or (iii) together with b form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo; and
b: (i) is selected from a group consisting of H, halogen, CH₃, CHF₂, CF₃, OCH₃, OCHF₂, OCF₃, SCH₃, SCHF₂, SCF₃, and cyano; or (ii) together with a form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo; or (iii) together with R² form a chain of 3 or 4 atoms, one atom of which is selected from the group consisting of C, N, O, and S, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₃-C₆ cycloalkyl, CHF₂, CF₃, OCHF₂, OCF₃, SCH₃, SCHF₂, SCF₃, cyano, and oxo;
   wherein:
R³: (i) is selected from the group consisting of H, C₁-C₆ alkyl, C₂-C₆ alkenyl, C₂-C₆ alkynyl, C₃-C₆ cycloalkyl, (C₃-C₆ cycloalkyl)(C₁-C₆ alkyl), aryl(C₁-C₆ alkyl), acetyl, and heteroaryl(C₁-C₆ alkyl); or (ii) together with R⁴ and the N atom to which they are attached form a 4-7 membered heterocyclyl ring; or (iii) together with f and the N atom to which R³ is attached form an azetidine or pyrrolidine ring, such ring carrying substituents independently selected from the group consisting of H, aryl, heteroaryl, C₁-C₆ alkyl, and C₃-C₆ cycloalkyl; or (iv) together with c and the N atom to which R³ is attached form an azetidine or pyrrolidine ring, such ring carrying substituents independently selected from the group consisting of H, aryl, heteroaryl, halogen, C₁-C₆ alkyl, and C₃-C₆ cycloalkyl;
R⁴: (i) is selected from the group consisting of H, C₁-C₆ alkyl, C₂-C₆ alkenyl, C₂-C₆ alkynyl, C₃-C₆ cycloalkyl, (C₃-C₆ cycloalkyl)(C₁-C₆ alkyl), aryl(C₁-C₆ alkyl), acetyl, and heteroaryl(C₁-C₆ alkyl); or (ii) together with R³ and the N atom to which they are attached form a 4-7 membered heterocyclyl ring; and
   wherein:
c, d, e, and f are each H; or three of c, d, e, and f are H and the remaining substituent is a lower alkyl group; or c and f are each H, and d and e together are —CH₂— or —CH₂CH₂—, thereby giving rise to a cyclopropane or cyclobutane ring; or c, d, and e are each H, and f, R³, and the N atom to which R³ is attached form together an azetidine or pyrrolidine ring, such ring carrying substituents independently selected from the group consisting of H, aryl, heteroaryl, C₁-C₆ alkyl, and C₃-C₆ cycloalkyl; or d, e, and f are each H, and c, R³, and the N atom to which R³ is attached form together an azetidine or pyrrolidine ring, such ring carrying substituents independently selected from the group consisting of H, aryl, heteroaryl, halogen, C₁-C₆ alkyl, and C₃-C₆ cycloalkyl; or d, e, and f are each H, and c and Z together comprise 1 or 2 carbon atoms so as to give rise to a pyran or oxepan ring, such ring carrying substituents independently selected from the group consisting of H, halogen, C₁-C₆ alkyl, and C₃-C₆ cycloalkyl.

### Examples of Embodiments of Chemical Entities

Examples of chemical entities of Formula I are shown in Table 1 below.

TABLE 1
Examples of Compounds of Formula I *

| | 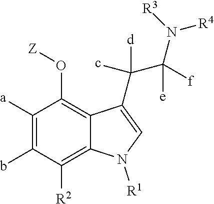 Formula I | | | | | | | | | | |
|---|---|---|---|---|---|---|---|---|---|---|---|
| cpd# | R¹ | R² | b | a | Z | c | d | e | f | R³ | R⁴ |
| 1 | H | Me | H | H | H | H | H | H | H | Me | Me |
| 2 | Me | Me | H | H | H | H | H | H | H | Me | Me |
| 3 | CD₃ | Me | H | H | H | H | H | H | H | Me | Me |
| 4 | H | Me | H | H | H | D | D | D | D | Me | Me |
| 5 | Me | Et | H | H | H | H | H | H | H | CD₃ | CD₃ |
| 6 | CD₃ | CD₃ | H | H | H | D | D | D | D | CD₃ | CD₃ |
| 7 | H | (CH₂)₂OH | H | H | H | H | H | H | H | Me | Me |
| 8 | H | (CH₂)₂F | H | H | H | H | H | H | H | Me | Me |
| 9 | H | Et | H | H | H | H | H | H | H | Me | Me |
| 10 | H | i-Pr | H | H | H | H | H | H | H | Me | Me |
| 11 | Me | i-Pr | H | H | H | H | H | H | H | Me | Me |
| 12 | (CH₂)₃ | | H | H | H | H | H | H | H | Me | Me |
| 13 | H | Me | H | H | H | H | H | H | Me | H | Cypr |
| 14 | H | Me | H | H | H | H | H | H | H | Me | Et |
| 15 | H | Me | H | H | H | H | H | H | H | Me | n-Pr |
| 16 | H | Me | H | H | H | H | H | H | H | Me | i-Pr |
| 17 | H | Me | H | H | H | H | H | H | H | Me | Cypr |
| 18 | H | Me | H | H | H | H | H | H | H | Me | CyprCH₂ |
| 19 | H | Me | H | H | H | H | H | H | H | Et | Et |
| 20 | H | Me | H | H | Ac | H | H | H | H | Me | Me |
| 21 | Me | Me | H | H | Piv | H | H | H | H | Me | Me |
| 22 | H | Me | H | H | Z¹ | H | H | H | H | Me | Me |
| 23 | Me | Me | H | H | Z² | H | H | H | H | Me | Me |
| 24 | H | Me | H | H | Z³ | H | H | H | H | Me | Me |
| 25 | Me | Me | H | H | Z⁴ | H | H | H | H | Me | Me |
| 26 | H | Me | H | H | Me | H | H | H | H | Me | Me |
| 27 | H | CD₃ | H | H | Me | H | H | H | H | Me | Me |
| 28 | H | Bn | H | H | H | H | H | H | H | Me | Me |
| 29 | Et | Me | H | H | H | H | H | H | H | Me | Me |
| 30 | H | Me | H | F | H | H | H | H | H | Me | Me |
| 31 | Me | Me | H | Cl | H | H | H | H | H | Me | Me |
| 32 | H | Me | F | H | H | H | H | H | H | H | Me |
| 33 | H | Me | F | H | H | H | H | H | H | Me | Me |
| 34 | H | Me | F | H | Me | H | H | H | H | Me | Me |
| 35 | H | Me | Cl | H | H | H | H | H | H | Me | Me |
| 36 | H | H | F | H | H | H | H | H | H | Me | Me |
| 37 | H | H | Me | H | H | H | H | H | H | Me | Me |
| 38 | H | Me | F | H | Bn | D | D | D | D | Me | Me |
| 39 | H | Me | H | H | F₃CBn | H | H | H | H | Me | Me |
| 40 | H | Me | H | CH₂CH₂ | | H | H | H | H | Me | Me |
| 41 | H | Me | H | CH₂CH₂CH₂ | | H | H | H | H | Me | Me |
| 42 | H | Me | H | CH═CH | | H | H | H | H | Me | Me |
| 43 | H | Me | H | CH₂C(O) | | H | H | H | H | Me | Me |
| 44 | H | Me | H | OCH₂ | | H | H | H | H | Me | Me |
| 45 | H | Me | CHF₂ | H | H | H | H | H | H | Me | Me |
| 46 | H | Me | CF₃ | H | H | H | H | H | H | Me | Me |
| 47 | H | Me | CN | H | H | H | H | H | H | Me | Me |
| 48 | H | Me | OMe | H | H | H | H | H | H | Me | Me |
| 49 | H | Me | OMe | H | H | H | H | H | H | H | Me |
| 50 | H | Me | H | H | H | H | H | H | H | H | ClBn |
| 51 | H | n-Pr | H | H | H | H | H | H | H | H | MeBn |
| 52 | H | Me | H | H | H | H | CH₂ | | H | Me | Me |
| 53 | Me | Me | H | H | H | H | CH₂CH₂ | | H | Me | Me |
| 54 | H | Me | H | H | CH₂ | | H | H | H | Me | Me |
| 55 | Me | Me | H | H | CH₂CH₂ | | H | H | H | Me | Me |
| 56 | H | Me | H | H | H | H | H | H | H | CH₂CH₂CH₂ | |
| 57 | H | Me | H | H | H | H | H | H | CH₂CH₂ | | H |
| 58 | H | Me | H | H | H | H | H | H | CH₂CH₂CH₂ | | H |
| 59 | H | Me | H | H | H | H | H | H | CH₂CH₂ | | Me |
| 60 | H | Me | H | H | H | H | H | H | CH₂CH₂CH₂ | | Me |
| 61 | H | FPh | H | H | H | H | H | H | H | Me | Me |
| 62 | H | Me | H | H | Ph | H | H | H | H | Me | Me |
| 63 | H | CF₃ | H | H | H | H | H | H | H | Me | Me |
| 64 | H | CN | H | H | H | H | H | H | H | Me | Me |
| 65 | H | C(O)NH₂ | H | H | H | H | H | H | H | Me | Me |
| 66 | H | 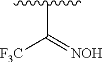 | H | H | H | H | H | H | H | Me | Me |
| 67 | H | (CH₂)₃ | | H | H | H | H | H | H | Me | Me |
| 68 | H | CH₂CH₂O | | H | H | H | H | H | H | Me | Me |
| 69 | H | CH═CHO | | H | H | H | H | H | H | Me | Me |
| 70 | H | (CH₂)₃O | | H | H | H | H | H | H | Me | Me |
| 71 | H | ON═C(CF₃) | | H | H | H | H | H | H | Me | Me |
| cpd# | R¹ | R² | b | a | Z | c | d | R³ | R⁴ | f | e |

| | | | | | | | | | | |
|---|---|---|---|---|---|---|---|---|---|---|
| 72 | H | Me | H | H | H | H | H | Me | CH₂CH₂ | H |
| 73 | H | Me | F | H | H | H | H | Me | CH₂CH₂ | H |

| | | | | | | | | | | |
|---|---|---|---|---|---|---|---|---|---|---|
| 74 | H | Me | H | H | H | H | CH₂ | H | H | H |
| 75 | H | Me | H | H | H | H | CH₂CH₂ | H | H | H |
| 76 | H | Me | H | H | H | H | CH₂ | Me | H | H |
| 77 | H | Me | H | H | H | H | CH₂CH₂ | Me | H | H |

*Abbreviations used in the table above have the following meanings: Ac = acetyl; Bn = benzyl; Bu = butyl; ClBn = m-chlorobenzyl; Cypr = cyclopropyl; Me = methyl; Et = ethyl; FPh = p-fluorophenyl; MeBn = m-methylbenzyl; Ph = phenyl; Piv = pivaloyl; Pr = propyl; Z¹ = C(O)N(H)-i-Pr; Z² = C(O)NMe₂; Z³ = P(O)(OH)₂; Z⁴ = P(O)(ONa)₂.

Examples of chemical entities of Formula II are shown in Table 2 below.

TABLE 2
Examples of Compounds of Formula II *

| | 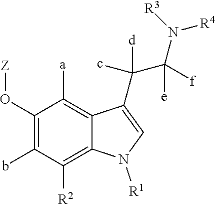 Formula II | | | | | | | | | | |
|---|---|---|---|---|---|---|---|---|---|---|---|
| cpd# | R¹ | R² | b | Z | a | c | d | e | f | R³ | R⁴ |
| 78 | H | Me | H | H | H | H | H | H | H | Me | Me |
| 79 | Me | Me | H | H | H | H | H | H | H | Me | Me |
| 80 | CD₃ | Me | H | H | H | H | H | H | H | Me | Me |
| 81 | H | Me | H | H | H | D | D | D | D | Me | Me |
| 82 | Me | Et | H | H | H | H | H | H | H | CD₃ | CD₃ |
| 83 | CD₃ | CD₃ | H | H | H | D | D | D | D | CD₃ | CD₃ |
| 84 | H | n-Bu | H | H | H | H | H | H | H | Me | Me |
| 85 | H | i-Pr | H | H | H | H | H | H | H | Me | Me |
| 86 | Me | i-Pr | H | H | H | H | H | H | H | Me | Me |
| 87 | (CH₂)₃ | | H | H | H | H | H | H | H | Me | Me |
| 88 | H | Me | H | H | H | H | H | H | Me | H | Cypr |
| 89 | H | Me | H | Ac | H | H | H | H | H | Me | Me |
| 90 | Me | Me | H | Piv | H | H | H | H | H | Me | Me |
| 91 | H | Me | H | Z¹ | H | H | H | H | H | Me | Me |
| 92 | Me | Me | H | Z² | H | H | H | H | H | Me | Me |
| 93 | H | Me | H | Z³ | H | H | H | H | H | Me | Me |
| 94 | Me | Me | H | Z⁴ | H | H | H | H | H | Me | Me |
| 95 | H | Me | H | H | H | H | H | H | H | Me | Et |
| 96 | H | Me | H | H | H | H | H | H | H | Me | n-Pr |
| 97 | H | Me | H | H | H | H | H | H | H | Me | i-Pr |
| 98 | H | Me | H | H | H | H | H | H | H | Me | Cypr |
| 99 | H | Me | H | H | H | H | H | H | H | Me | CyprCH₂ |
| 100 | H | Me | H | H | H | H | H | H | H | Et | Et |
| 101 | H | Bn | H | H | H | H | H | H | H | Me | Me |
| 102 | Et | Me | H | H | H | H | H | H | H | Me | Me |
| 103 | H | Me | H | CD₃ | H | H | H | H | H | Me | Me |
| 104 | Me | Me | H | H | Cl | H | H | H | H | Me | Me |
| 105 | H | Me | F | H | H | H | H | H | H | Me | Me |
| 106 | H | Me | F | Bn | H | D | D | D | D | Me | Me |
| 107 | H | Me | H | CH₂CH₂ | | H | H | H | H | Me | Me |
| 108 | H | Me | H | CH(Me)CH₂ | | H | H | H | H | Me | Me |
| 109 | H | Me | H | CH₂CH₂CH₂ | | H | H | H | H | Me | Me |
| 110 | H | Me | H | CH₂CH₂CH₂ | | H | H | H | H | H | H |
| 111 | H | Me | H | CH═CH | | H | H | H | H | Me | Me |
| 112 | H | Me | CHF₂ | H | H | H | H | H | H | Me | Me |
| 113 | Me | Me | CF₃ | H | H | H | H | H | H | Me | Me |
| 114 | H | Me | H | H | H | H | H | H | H | H | ClBn |
| 115 | H | n-Pr | H | H | H | H | H | H | H | H | MeBn |
| 116 | H | Me | H | H | H | H | CH₂ | | H | Me | Me |
| 117 | Me | Me | H | H | H | H | CH₂CH₂ | | H | Me | Me |
| 118 | H | Me | H | H | CH₂CH₂ | | H | H | H | Me | Me |
| 119 | Me | Me | H | H | CH₂CH₂CH₂ | | H | H | H | Me | Me |
| 120 | H | Me | H | H | CH₂OCH₂ | | H | H | H | Me | Me |
| 121 | H | Me | H | H | H | H | H | H | H | CH₂CH₂CH₂ | |
| 122 | H | Me | H | H | H | H | H | H | CH₂CH₂ | | H |
| 123 | H | Me | H | H | H | H | H | H | CH₂CH₂CH₂ | | H |
| 124 | H | Me | H | H | H | H | H | H | CH₂CH₂ | | Me |
| 125 | H | Me | H | H | H | H | H | H | CH₂CH₂CH₂ | | Me |
| 126 | H | FPh | H | H | H | H | H | H | H | Me | Me |
| 127 | H | 2-Py | H | H | H | H | H | H | H | Me | Me |
| 128 | H | Me | H | Ph | H | H | H | H | H | Me | Me |
| cpd# | R¹ | R² | b | Z | a | c | d | R³ | R⁴ | f | e |

| | | | | | | | | | | |
|---|---|---|---|---|---|---|---|---|---|---|
| 129 | H | Me | H | H | H | H | H | Me | CH₂CH₂ | H |
| 130 | H | Me | F | H | H | H | H | Me | CH₂CH₂ | H |

| | | | | | | | | | | |
|---|---|---|---|---|---|---|---|---|---|---|
| 131 | H | Me | H | H | H | H | CH₂ | H | H | H |
| 132 | H | Me | H | H | H | H | CH₂CH₂ | Me | H | H |
| 133 | H | Me | H | H | H | H | CH₂ | H | H | H |
| 134 | H | Me | H | H | H | H | CH₂CH₂ | Me | H | H |

*Abbreviations used in the table above have the following meanings: Ac = acetyl; Bn = mbenzyl; Bu = butyl; CIBn = m-chlorobenzyl; Cypr = cyclopropyl; Me = methyl; Et = ethyl; FPh = p-fluorophenyl; MeBn = m-methylbenzyl; Ph = phenyl; Piv = pivaloyl; Pr = propyl; Py = pyridyl; Z¹ = C(O)N(H)-i-Pr; Z² = C(O)NMe₂; Z³ = P(O)(OH)₂; Z⁴ = P(O)(ONa)₂.

Examples of chemical entities of Formula III are shown in Table 3 below.

TABLE 3
Examples of Compounds of Formula III *

| | 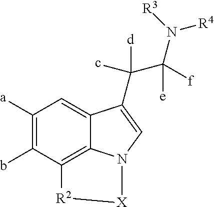 Formula II | | | | | | | | |
|---|---|---|---|---|---|---|---|---|---|
| cpd# | R² | a | b | c | d | e | f | R³ | R⁴ |
| 135 | O(CH₂)₂ | H | H | H | H | H | H | CH₃ | CH₃ |
| 136 | O(CH₂)₃ | H | H | H | H | H | H | CH₃ | CH₃ |
| 137 | S(CH₂)₂ | H | H | H | H | H | H | CH₃ | CH₃ |
| 138 | S(CH₂)₃ | H | H | H | H | H | H | CH₃ | CH₃ |

### Pharmacology

### Serotonin Receptor 5-HT₂ Functional Assays

Non-limiting examples of methods of measuring serotonin receptor functional activation are described as follows.

To measure serotonin receptor functional activation, either Gq dissociation by bioluminescence resonance energy transfer (BRET) or Gq-dependent calcium flux was performed for selected compounds. To measure 5-HT₂ receptor-mediated Gq activation via Gq/γ1 dissociation as measured by BRET (McCorvy J D, Wacker D, Wang S, Agegnehu B, Liu J, Lansu K, Tribo A R, Olsen R H J, Che T, Jin J, Roth B L. Structural determinants of 5-HT2B receptor activation and biased agonism. Nat Struct Mol Biol. 2018; 25(9):787-96), HEK293T cells were sub-cultured in Dulbecco's Modified Eagle Medium (DMEM) supplemented with 10% dialyzed fetal bovine serum (FBS) and were co-transfected in a 1:1:1:1 ratio with RLuc8-fused human Gαq (σaq-RLuc8), a GFP²-fused to the C-terminus of human Gγ1(Gγ1-GFP²), human Gβ1, and 5-HT₂ receptor using TransiT-2020. After at least 18-24 hours, transfected cells were plated in poly-lysine coated 96-well white clear bottom cell culture plates in DMEM containing 1% dialyzed FBS at a density of 25,000-40,000 cells in 200 μL per well and incubated overnight. The next day, medium was decanted, and cells were washed with 60 μL of drug buffer (1×HBSS, 20 mM HEPES, pH 7.4), then 60 μL of drug buffer was added per well. Cells were pre-incubated in a humidified atmosphere at 37° C. before receiving drug stimulation. Drug stimulation utilized 30 μL addition of drug (3×) diluted in McCorvy buffer (1×HBSS, 20 mM HEPES, pH 7.4, supplemented with 0.3% BSA fatty acid free, 0.03% ascorbic acid), and plates were incubated for 1 hour at 37° C. Substrate addition occurred 15 minutes before reading and utilized 10 μL of the RLuc substrate coelenterazine 400a for Gq dissociation BRET² (Prolume/Nanolight, 5 μM final concentration). Plates were read for luminescence at 400 nm and fluorescent GFP² emission at 510 nm at 1 second per well using a Mithras LB940 (multimode microplate reader (e.g. one provided by Berthold)). The BRET ratios of fluorescence/luminescence were calculated per well and were plotted as a function of drug concentration using Graphpad Prism 8 (Graphpad Software Inc., San Diego, CA). Data were normalized to % 5-HT stimulation and analyzed using nonlinear regression “log(agonist) vs. response” to yield Eₘₐₓ and EC₅₀ parameter estimates.

Calcium flux was measured using stable-expressing 5-HT₂ Flp-In 293 T-Rex Tetracycline inducible system by methods known in the art (e.g. Investigation of the Structure-Activity Relationships of Psilocybin Analogues, ACS Pharmacol. Transl. Sci. 2020, Publication Date: Dec. 14, 2020, https://doi.org/10.1021/acsptsci.0c00176). Cell lines were maintained in DMEM containing 10% FBS, 10 μg/mL Blasticidin, and 100 μg/mL hygromycin B. At least 20-24 hours before the assay, receptor expression was induced with tetracycline (2 μg/mL), and cells were seeded into 384-well poly-L-lysine-coated black plates at a density of 7,500 cells/well in DMEM containing 1% dialyzed FBS. On the day of the assay, the cells were incubated for 1 hour at 37° C. with Fluo-4 Direct dye (Invitrogen, 20 μL/well) reconstituted in drug buffer (20 mM HEPES-buffered HBSS, pH 7.4) containing 2.5 mM probenecid. Drug dilutions were prepared at 5× final concentration in McCorvy buffer (20 mM HEPES-buffered HBSS, 0.1% BSA, 0.01% ascorbic acid, pH 7.4). After dye load, cells were allowed to equilibrate to room temperature for 15 minutes, and then placed in a FLIPR^{TETRA} fluorescence imaging plate reader (Molecular Devices). The FLIPR^{TETRA} was programmed to read baseline fluorescence for 10 s (1 read/s), and afterward 5 μL of drug per well was added, and fluorescence was read for a total of 5-10 min (1 read/s). Fluorescence in each well was normalized to the average of the first 10 reads for baseline fluorescence, and then either maximum-fold peak increase over baseline or area under the curve (AUC) was calculated. Either peak or AUC was plotted as a function of drug concentration, and data were normalized to percent 5-HT stimulation. Data were plotted, and non-linear regression was performed using “log(agonist) vs. response” in Graphpad Prism 8 to yield Eₘₐₓ and EC₅₀ parameter estimates.

The functional activity of various compounds disclosed herein at each of the 5-HT2A, 5-HT2B, and 5-HT2C receptors was measured against and relative to the functional activity of 4-hydroxytryptamine at those receptors. A comparison of the functional activities is provided in Table 4 as follows. At least three replicate trials were performed for each compound:

TABLE 4

| | | 5-HT2A | | 5-HT2B | | | |
|---|---|---|---|---|---|---|---|
| | | BRET | | BRET | | 5-HT2C BRET | |
| Compound | Identification | EC₅₀ | | EC₅₀ | | EC₅₀ | |
| — | — | (nM) | Eff % | (nM) | Eff % | (nM) | Eff % |
| 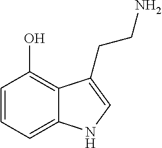 [SMILES: NCCc1c[nH]c2cccc(O)c12] | 4- hydroxytryptamine (reference compound) | 2.9 | 100 | 0.8 | 100 | 0.35 | 99 |
| 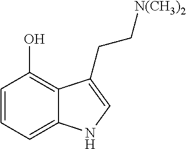 [SMILES: CN(C)CCc1c[nH]c2cccc(O)c12] | psilocin (reference compound) | 8.3 | 82 | 1.07 | 63 | 7.8 | 95 |
| 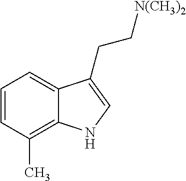 | 7,N,N-TMT (reference compound) | 232 | 80 | 74 | 36 | 11.7 | 79 |
| 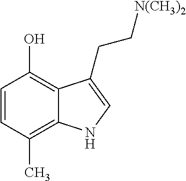 [SMILES: Cc1ccc(O)c2c(CCN(C)C)c[nH]c12] | Cpd #1 | 66 | 72 | inactive | | 6.9 | 79 |
| 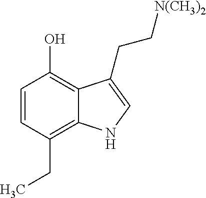 [SMILES: CCc1ccc(O)c2c(CCN(C)C)c[nH]c12] | Cpd #7 | 75 | 81 | inactive | | 5.6 | 76 |
| 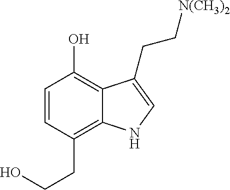 [SMILES: CN(C)CCc1c[nH]c2c(CCO)ccc(O)c12] | Cpd #8 | inactive | | inactive | | 3930 | 56 |
| 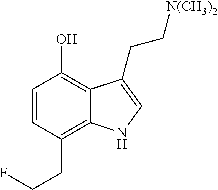 [SMILES: CN(C)CCc1c[nH]c2c(CCF)ccc(O)c12] | Cpd #9 | 154 | 78 | inactive | | 28 | 67 |
| 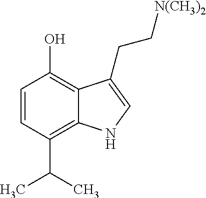 [SMILES: CC(C)c1ccc(O)c2c(CCN(C)C)c[nH]c12] | Cpd #10 | 1070 | 40 | inactive | | 34 | 60 |
| 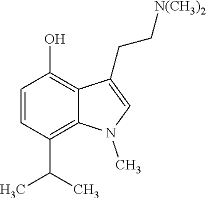 [SMILES: CC(C)c1ccc(O)c2c(CCN(C)C)cn(C)c12] | Cpd #11 | 1160 | 66 | inactive | | 22 | 88 |
| 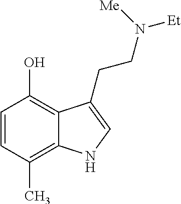 [SMILES: CCN(C)CCc1c[nH]c2c(C)ccc(O)c12] | Cpd #14 | 82 | 63 | inactive | | 4.2 | 108 |
| 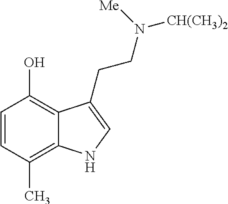 [SMILES: Cc1ccc(O)c2c(CCN(C)C(C)C)c[nH]c12] | Cpd #16 | 150 | 77 | inactive | | 53 | 103 |
| 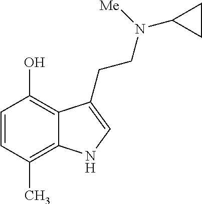 [SMILES: Cc1ccc(O)c2c(CCN(C)C3CC3)c[nH]c12] | Cpd #17 | 12.9 | 54 | inactive | | 3.2 | 111 |
| 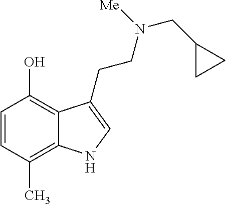 [SMILES: Cc1ccc(O)c2c(CCN(C)CC3CC3)c[nH]c12] | Cpd #18 | 92 | 76 | inactive | | 25 | 114 |
| 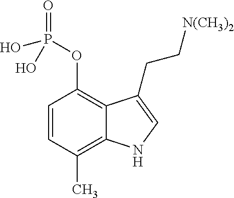 [SMILES: Cc1ccc(OP(=O)(O)O)c2c(CCN(C)C)c[nH]c12] | Cpd #24 | 1150 | 69 | inactive | | 370 | 69 |
| 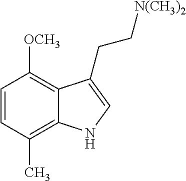 [SMILES: COc1ccc(C)c2[nH]cc(CCN(C)C)c12] | Cpd #26 | 95 | 68 | inactive | | 3.5 | 113 |
| 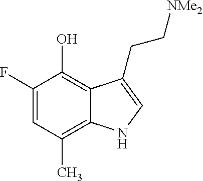 [SMILES: Cc1cc(F)c(O)c2c(CCN(C)C)c[nH]c12] | Cpd #30 | 13.4 | 64 | 0.2 | | 8 | 1.6 |
| 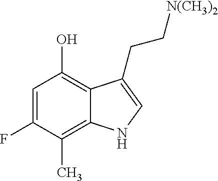 [SMILES: Cc1c(F)cc(O)c2c(CCN(C)C)c[nH]c12] | Cpd #33 | 20 | 78 | inactive | | 1.4 | 102 |
| 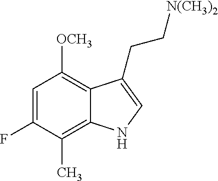 [SMILES: COc1cc(F)c(C)c2[nH]cc(CCN(C)C)c12] | Cpd #34 | 16.9 | 84 | inactive | | 1.8 | 94 |
| 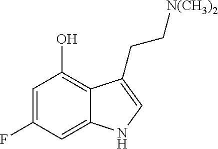 [SMILES: CN(C)CCc1c[nH]c2cc(F)cc(O)c12] | Cpd #36 | 2.9 | 92 | 4.2 | 62 | 1.7 | 93 |
| 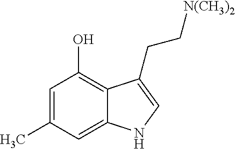 [SMILES: Cc1cc(O)c2c(CCN(C)C)c[nH]c2c1] | Cpd #37 | 59 | 88 | 189 | 41 | 1.5 | 95 |
| 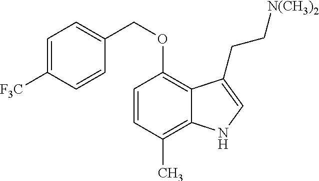 [SMILES: Cc1ccc(OCc2ccc(C(F)(F)F)cc2)c2c(CCN(C)C)c[nH]c12] | Cpd #39 | 7660 | 90 | inactive | | 131 | 81 |
| 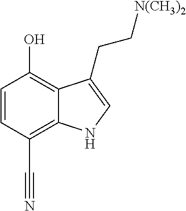 [SMILES: CN(C)CCc1c[nH]c2c(C#N)ccc(O)c12] | Cpd #64 | 267 | 49 | inactive | | 88 | 79 |
| 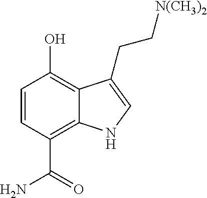 [SMILES: CN(C)CCc1c[nH]c2c(C(N)=O)ccc(O)c12] | Cpd #65 | inactive | | inactive | | 9960 | 69 |
| 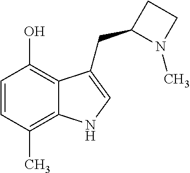 [SMILES: Cc1ccc(O)c2c(C[C@H]3CCN3C)c[nH]c12] | Cpd #77 (in the depicted enantiomeric form) | 49 | 69 | 57 | 41 | 7.3 | 66 |
| 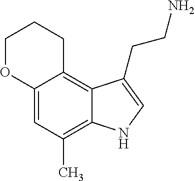 [SMILES: Cc1cc2c(c3c(CCN)c[nH]c13)CCCO2] | Cpd #110 | 277 | 91 | 139 | 63 | 69 | 96 |
| 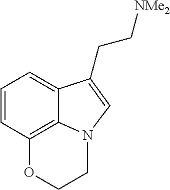 [SMILES: CN(C)CCc1cn2c3c(cccc13)OCC2] | Cpd #135 | 767 | 76 | 10.7 | 42 | 9 | 96 |

The compounds in Table 4 suggest that substitution of the 7-position on the indole ring leads to a corresponding decrease in functional activity at the 5-HT_{2B} receptor.

The functional activity of the Cpd #1 compound at other receptor sites was also measured against and relative to the functional activity of psilocin at those receptors. A comparison of the functional activities is provided in Table 5 as follows.

TABLE 5

| | 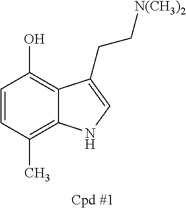 [SMILES: Cc1ccc(O)c2c(CCN(C)C)c[nH]c12] | | 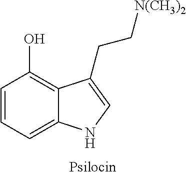 [SMILES: CN(C)CCc1c[nH]c2cccc(O)c12] | |
|---|---|---|---|---|
| | EC₅₀ (nM) | Eff % | EC₅₀ (nM) | Eff % |
| 5-HT1A | 12,600 | 76 | 46.9 | 93 |
| 5-HT1B | 309 | 105 | 14.3 | 94 |
| 5-HT1D | 749 | 112 | 2.2 | 102 |
| 5-HT1E | 167 | 102 | 10.4 | 90 |
| 5-HT1F | 23 | 92 | 9.3 | 93 |
| 5-HT2A | 66 | 73 | 8.3 | 82 |
| 5-HT2B | 3.6 | 16 | 1.1 | 63 |
| 5-HT2C | 6.9 | 79 | 7.8 | 95 |
| 5-HT4 | 257 | 13 | 563 | 81 |
| 5-HT5A | 23,000 | 20 | 349 | 48 |
| 5-HT6 | 9.0 | 48 | 183 | 74 |
| 5-HT7 | 5.2 | 3.4 | 525 | 86 |

Functional activity at the 5-HT_{1F} receptor has been reported to be relevant in the treatment of migraine. Functional activity at the 5-HT₆ receptor has been reported to be relevant in the treatment of cognitive disorders (e.g. dementia, Alzheimer's disease).

### Methods of Chemical Synthesis

Non-limiting examples of procedures for preparing the compounds described herein are provided below.

The indole core of the compounds of Formula I is numbered as follows:

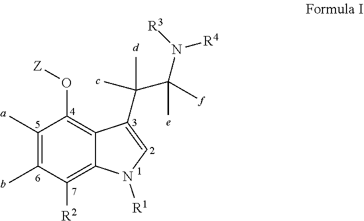

Analogous numbering applies for the compounds of Formula II, as the numbers are attached to the indole ring regardless of its substituents. The synthesis of these compounds comprises several steps, namely: (1) construction of the heterocyclic core (if not commercially available); (2) installation or modification of the 3-substituent; and (3) functional group transformations.

Indole compounds may be prepared using a variety of methods. Some indoles may be synthesized, for example, through the Leimgruber-Batcho indole synthesis and its modifications, as shown by way of example below:

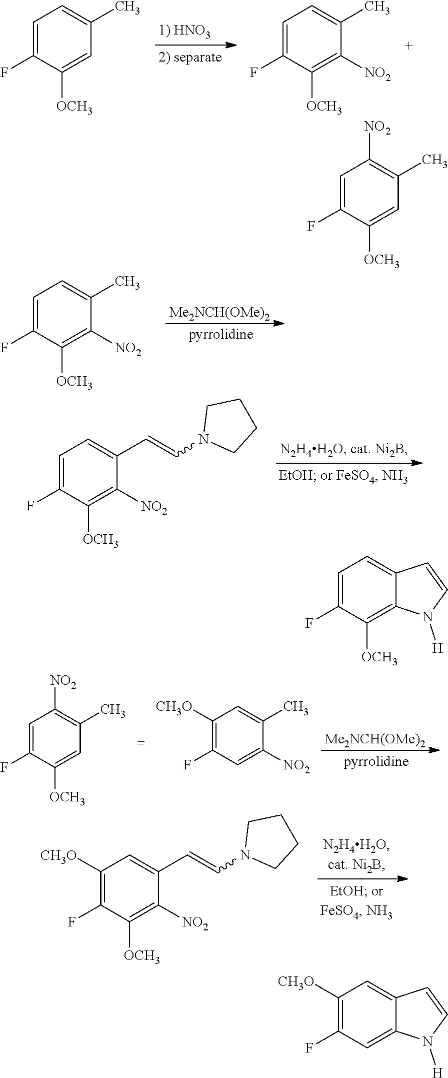

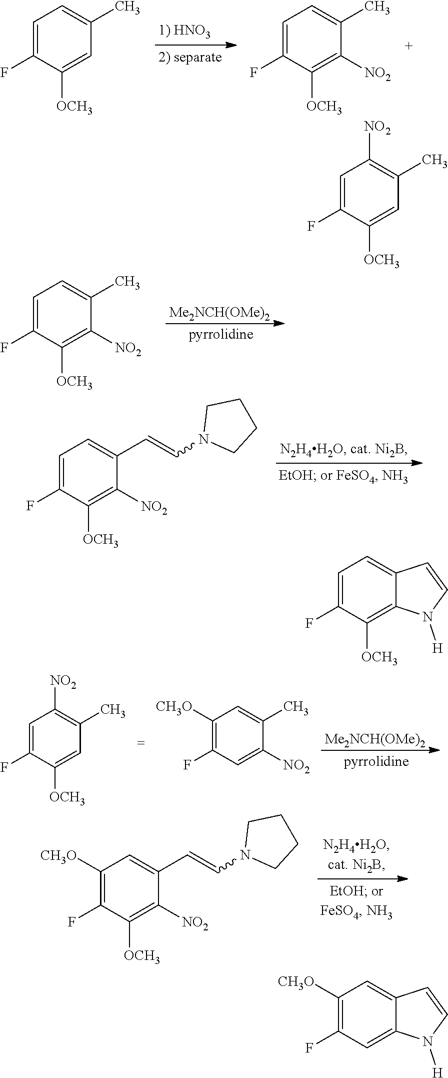

Substituted 2-nitrobenzaldehydes undergo a nitroaldol reaction with nitromethane to form substituted ortho,beta-dinitrostyrenes, whose reduction then delivers substituted indoles.

The N-alkylation of N-alkylanilines with ethyl 4-bromoacetoacetate followed by cyclization with ZnCl₂ represents another versatile approach of indole synthesis, as shown by way of example below:

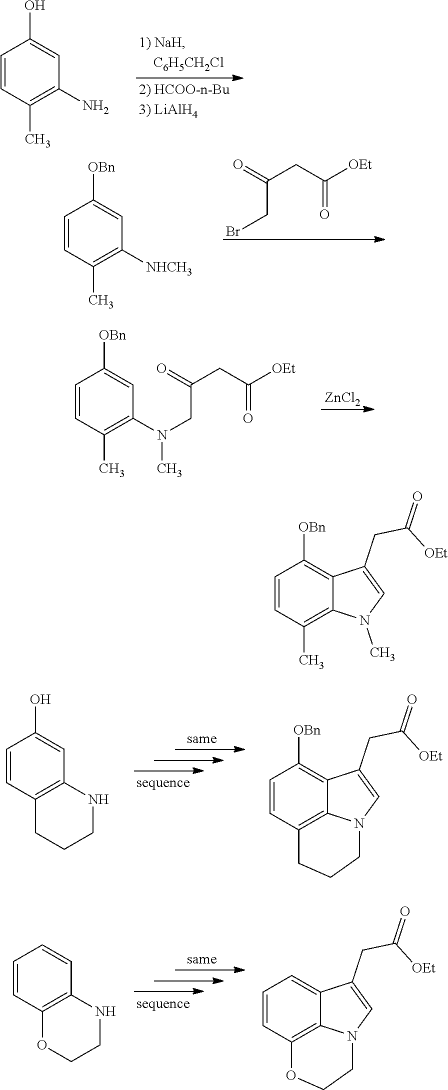

Yet other indoles may be prepared through a Bartoli reaction, as shown byway of example below:

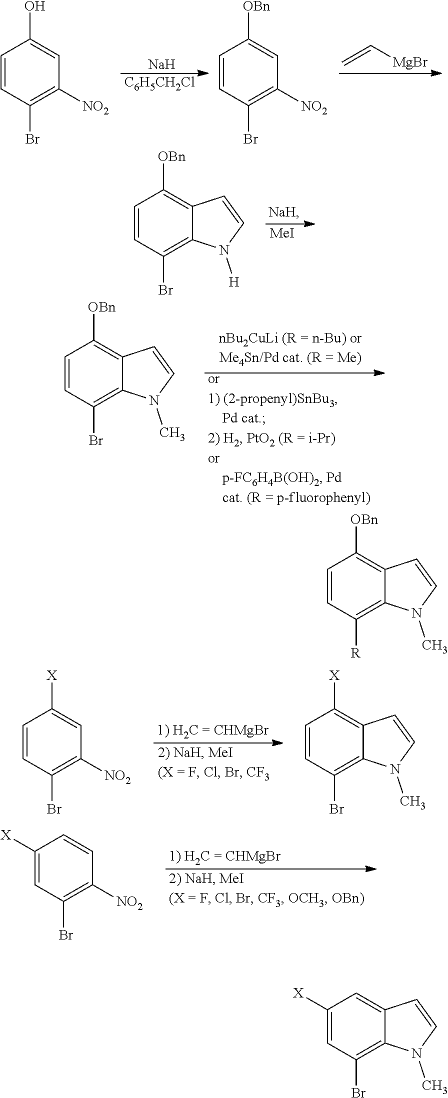

Methods for side chain installation and/or modification may also be used. If no substituent is present in the 3-position, indoles (with or without a 1-alkyl group) may be acylated with oxalyl chloride, the remaining second acyl halide function aminolyzed, and the carbonyl groups reduced to methylene groups with LiAlH₄ or borane. Products of partial reduction retaining a hydroxyl group in the benzylic position relative to the indole ring may be encountered as byproducts, and such products may be removed chromatographically by virtue of their higher polarity and may furthermore be converted to additional fully reduced material by catalytic hydrogenolysis or (especially in the presence of additional hydrogenolyzable functionality such as OBn) with Et₃SiH/CF₃COOH. Use of the deuterated form of the reducing agent gives access to compounds with a tetradeuterated side chain, as shown by way of example below:

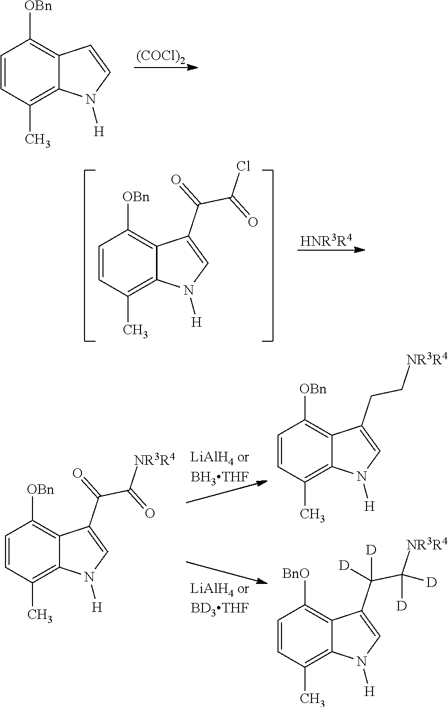

In another approach, the 3-position is formylated using the Vilsmeier-Haack protocol, the side chain is extended by one carbon atom with a Wittig reagent, and the resulting indolylacetaldehyde is reductively aminated, as shown by way of example below:

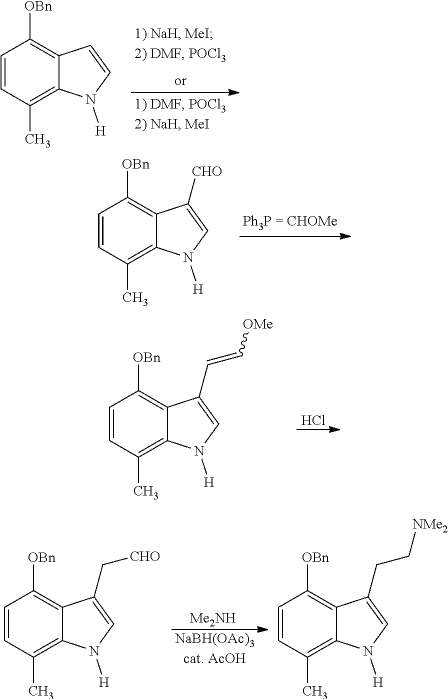

It is also feasible to use an (alkoxycarbonylmethylene)triphenylphosphorane as a Wittig reagent. After saturation of the double bond, the additional carbon atom is replaced with nitrogen, for example, by Hofmann degradation of the derived amide or, as shown, through a Curtius reaction. As an alternative to reductive alkylation with an aldehyde, the amino group may be acylated, followed by amide reduction with a hydride reagent such as LiAlH₄ or borane-THF. The use of a deuterated reducing agent in this step offers another handle for the deuteration of certain compounds disclosed herein. It is also possible to further alkylate the acylated nitrogen by deprotonation and reaction with, for example, an alkyl halide or sulfonate. Reduction of the alkylated amide as before leads to compounds bearing a dialkylated side chain N atom.

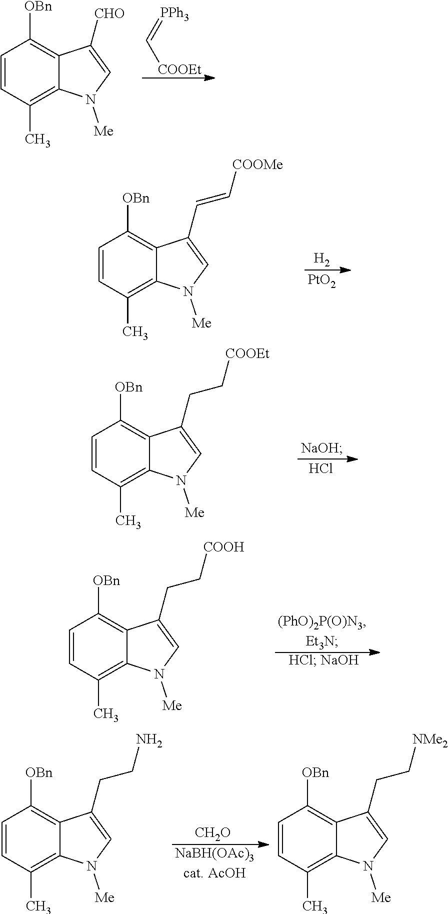

In another synthetic sequence, an indole-3-carboxaldehyde is chain-extended through a nitroaldol reaction. The nitrovinyl group is exhaustively reduced to aminoethyl, for example, with LiAlH₄, or by catalytic hydrogenation if the remaining functionality present in the compound permits. In the present scheme, N-alkylation of the amino group is performed indirectly by acylation followed by reduction of amide to amine.

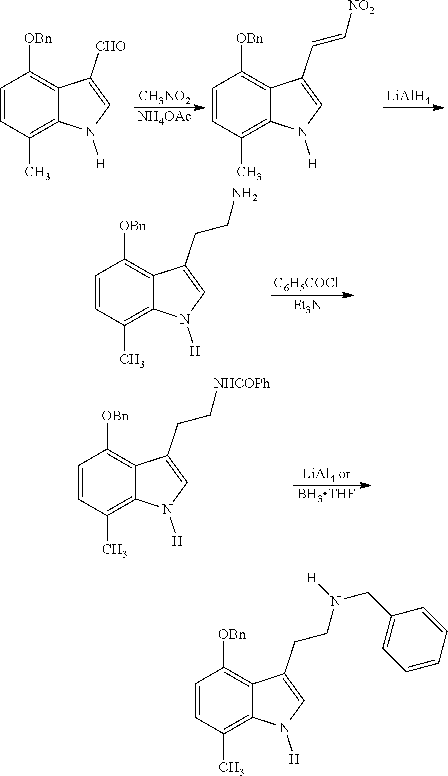

Certain synthetic methods for the preparation of indoles provide these in the form of their 3-acetic acids or esters thereof. Acids may be esterified by a variety of procedures, and the esters then aminolyzed by treatment with an amine in a polar solvent. Esters may conversely be hydrolyzed to acids, and the acids transformed to amides by treatment with an activating agent, many of which are known from the art of peptide synthesis, and the appropriate amine. The amides resulting from either procedure are then reduced to amines with a reactive hydride, such as LiAlH₄ or borane-THF. This sequence is suited for the synthesis of compounds containing a partially or completely deuterated ethylene (CH₂CH₂) moiety, by base-catalyzed H-D exchange adjacent to the amide carbonyl group and/or by employing deuterated hydride reagents in the reduction step.

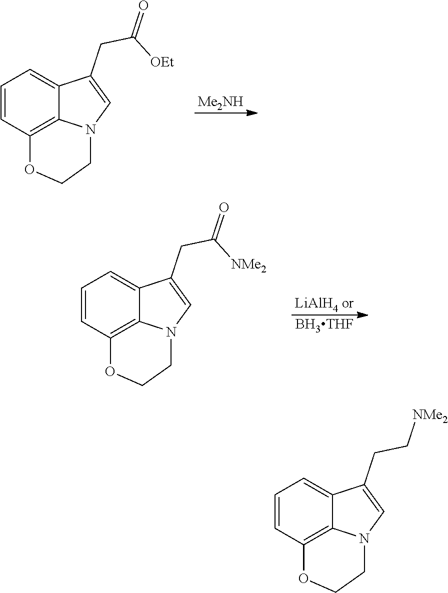 [SMILES: "Me2NH" ; CCOC(=O)Cc1cn2c3c(cccc13)OCC2 ; CN(C)C(=O)Cc1cn2c3c(cccc13)OCC2 ; "LiAlH4 or" ; CN(C)CCc1cn2c3c(cccc13)OCC2 ; "BH3·THF"]

Cyclopropane-containing side chains may be obtained by Kulinkovich aminocyclopropanation, as shown by way of example below:

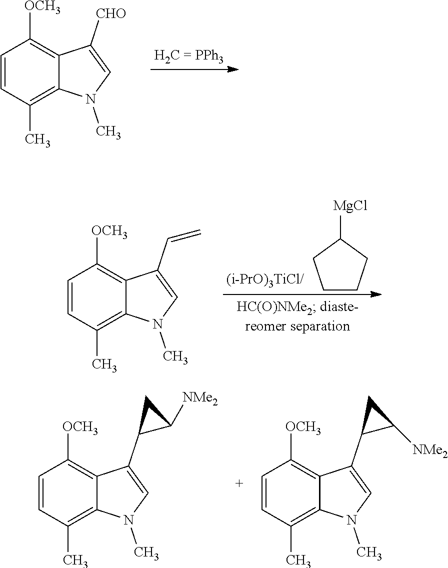

The cyclopropanes are formed as a mixture of two diastereoisomers (cis and trans), each of which is composed of equal amounts of its enantiomers. The diastereoisomers, and for each diastereoisomer its enantiomers, may be separated using standard techniques, such as crystallization, crystallization of diastereoisomeric salts with homochiral acids, chromatography, or chromatography on chiral stationary phases.

To prepare compounds whose side chains contain 2-substituted azetidine or pyrrolidine rings, a substituted indole is deprotonated with ethylmagnesium bromide. N-Boc-azetidine-2-carboxylic acid and N-Boc-proline are commercially available in both enantiomeric forms; in the accompanying scheme, the S-isomer is shown. From these building blocks, the acid chlorides are formed in situ and then are reacted with the deprotonated indole to form a 3-acylated indole. Upon exhaustive reduction, an N-methylazetidine or -pyrrolidine is obtained, wherein the methyl group is derived from reduction of the Boc group. Removal of the Boc group prior to reduction produces, on the other hand, the secondary amine lacking an N-methyl group.

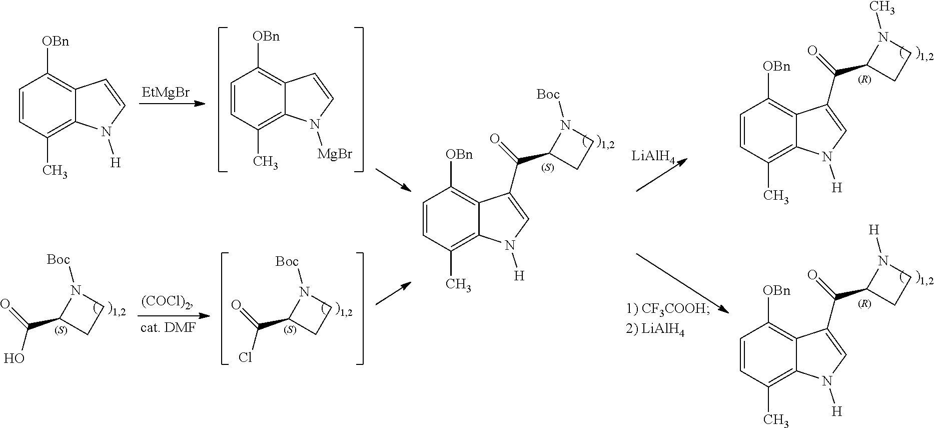

To prepare compounds whose side chains contain 3-substituted azetidine rings, a deprotonated or 3-lithiated substituted indole is alkylated with 1-azabicyclo[1.1.0]butane, prepared in situ from 2,3-dibromopropylamine hydrobromide.

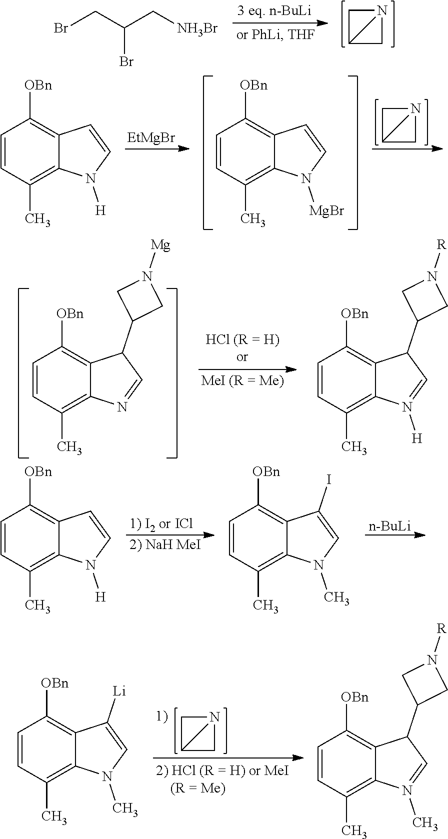

To prepare compounds whose side chains contain 3-substituted pyrrolidine rings, a substituted indole is alkylated with an N-substituted maleimide, followed by reduction with LiAlH₄, as shown by way of example below:

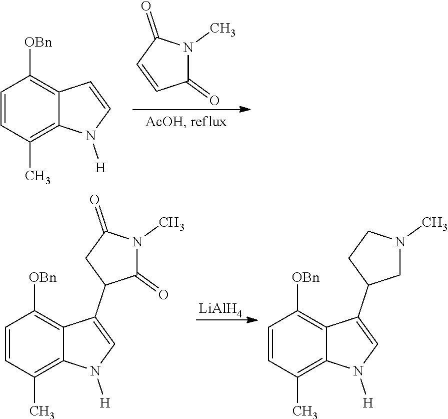

Additional and concluding functional group transformations may also be used herein. To effect (cyclo-)alkylation of the indole nitrogen, the indole is usually deprotonated and the alkyl group introduced as a halide (or sulfonate, or sulfate). Alternatively, a (cyclo-)alkylboronic acid or -boronic acid derivative can be employed in the presence of air as oxidant under copper catalysis (the Chan-Lam reaction). This protocol is also applicable to (cyclo-)alkyl groups whose corresponding halides or sulfonates are little or not electrophilic, such as cyclopropyl:

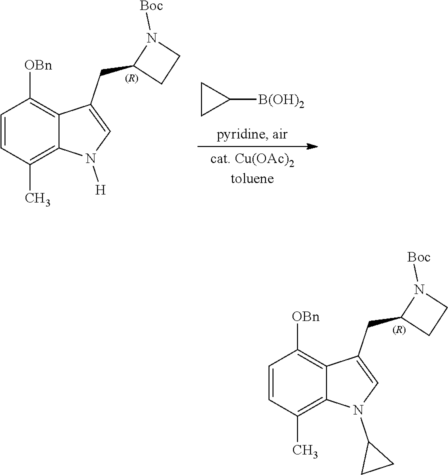

When a free hydroxyl group is desired on the indole ring, a widely applicable protected precursor is the benzyl ether. O-Benzyl groups are more readily hydrogenolyzed than benzyl groups at either the indole or side-chain nitrogen; undesired N-debenzylation can be limited or suppressed through proper choice of parameters such as hydrogen pressure, catalyst load, reaction temperature, reaction time, and solvent. If carbon-carbon double or triple bonds are present, these bonds are expected to undergo saturation concomitantly with O-debenzylation. If this is undesired, the O-benzyl groups can alternatively be removed by a variety of reagents, including Lewis acids such as boron tribromide, 2-bromo-1,3,2-benzodioxaborole, and bromodimethylborane. On the other hand, a particular type of (cyclo-)alkenyl electrophiles, namely allylic electrophiles, are far more reactive than their saturated analogs and can advantageously be employed to improve the yields achievable with (cyclo-)alkyl halides of lower reactivity, such as isobutyl iodide. Thus, methallyl bromide is used in its place, and the additional double bond is removed at the same time as the O-benzyl group; or either functionality susceptible to hydrogenation or hydrogenolysis over a Pd catalyst can be maintained through the choice of alternative reaction conditions while the other functionality is transformed:

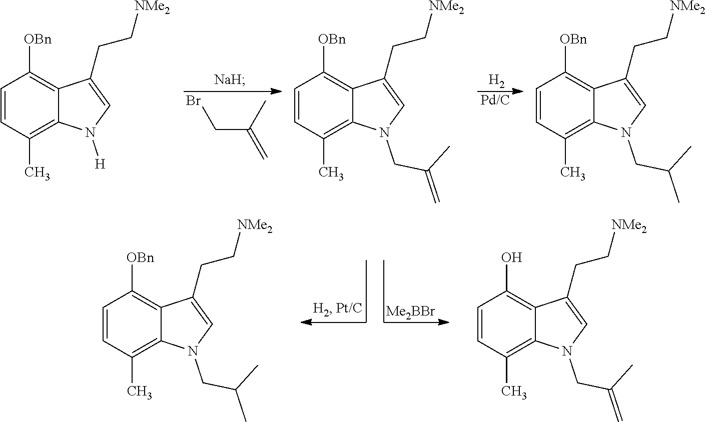

Similarly, 3-bromocyclopentene may serve as a precursor for an N-cyclopentyl group, and 3-bromocyclohexene for a cyclohexyl group. A leaving group on the 1-substituent may be used to effect ring formation with a hydroxyl group in position 7, as shown by way of example below:

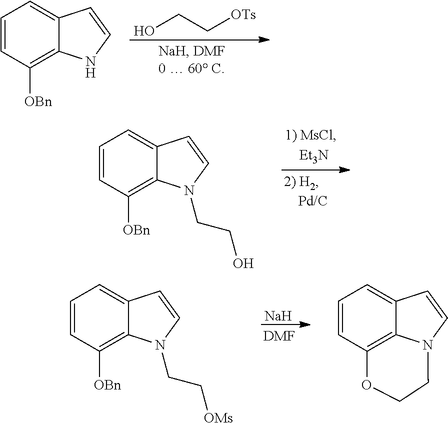

A common side reaction in the 1-alkylation of indoles with the 3-side chain already in place is overalkylation to form a quaternary ammonium salt. The excessive N-alkyl group, if it is the same as those already in place or is of greater reactivity than those, may be removed by treatment with a strong, soft nucleophile such as a thiolate anion, as shown by way of example below:

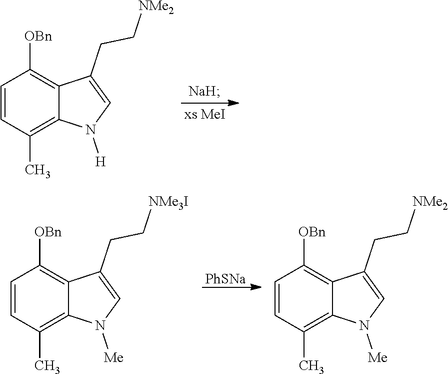

Extended reaction periods or elevated temperatures may also cause partial or complete removal of the phenol protecting group.

In some cases, indoles lacking a 7-substituent are readily available, and the subsequent introduction of a 7-substituent is a viable synthetic method to arrive at 7-substituted indoles. The literature (Hartung, C. G.; Fecher, A.; Chapell, B.; Snieckus, V. Org. Lett. 2003, 5, 1899) reports the implementation of this strategy for the example of 1-(diethylcarbamyl)indoles. The 2-position is the most reactive one towards metalation and is first blocked by silylation; the second metalation occurs in the 7-position, and the resulting organolithium intermediate can directly be alkylated as shown below:

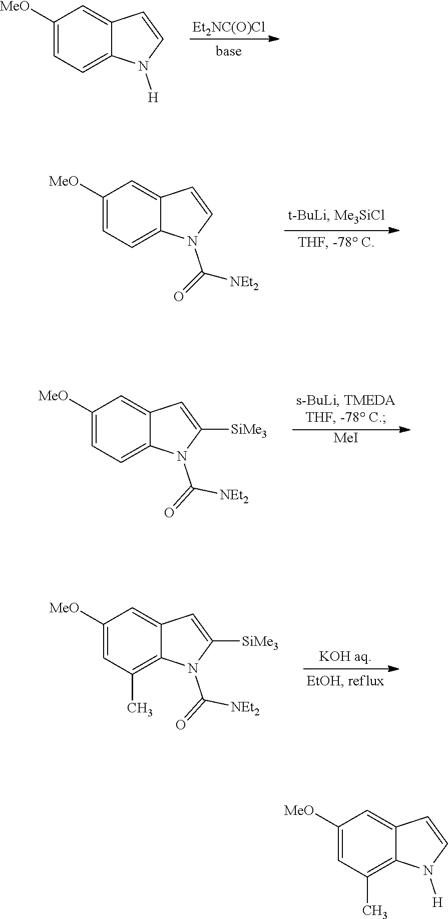

Removal of both the carbamyl and silyl groups is effected by treatment with KOH. If a halogenating agent (e.g., I₂ or BrCH₂CH₂Br) is used as the electrophile, the resulting aryl halide may be utilized as a reaction partner in transition-metal-catalyzed coupling reactions as already mentioned in the discussion of the Bartoli indole synthesis. 4,7- and 5,7-dibromoindole, available through the Bartoli synthesis, exhibit after 1-protection with a bulky acyl group differential reactivity of the two Br atoms towards halogen-metal exchange (Li, L.; Martins, A. Tetrahedron Lett. 2003, 44, 5987-5990). The 7-Br reacts selectively, and the resulting bromolithioindole can be trapped by addition of appropriate electrophiles. The 4- or 5-Br can be retained or utilized in further halogen-metal exchange or coupling reactions. Phenols can be obtained from metalated indoles by reaction with borate esters, followed by oxidation. With tert-butyl peroxybenzoate, metalated indoles react to form tert-butoxyindoles, which both represent specific embodiments of the invention as well as protected precursors (by acid treatment) of free phenols, thus offering an alternative to benzyl protecting groups.

The annulation of an oxygen heterocycle onto the benzene portion of the indole ring can be accomplished by appending the requisite additional carbon atoms through a Claisen rearrangement. The allyl migration from an oxygen in position 5 regioselectively occurs to the 4-position when available. Two additional steps are needed to cyclize the allylphenol intermediate to form a dihydropyran ring. As 7-substituted 5-hydroxyindoles are not at present commercially available, the introduction of the 7-substituent is in this case effected through the directed metalation approach introduced above.

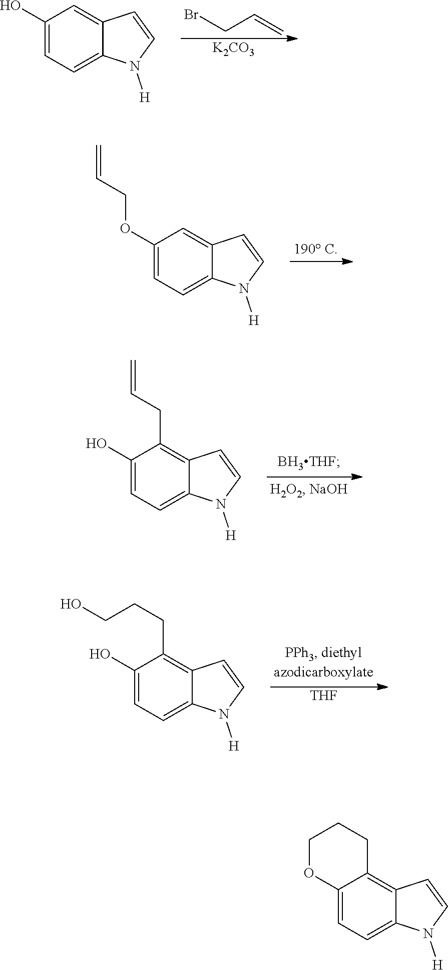

The corresponding propargyl ether leads directly to the depicted pyran through spontaneous cyclization of the intermediate allenylphenol. In this case, the introduction of an electron-withdrawing 3-formyl group, later serving as a handle for side chain installation, allows for a smoother rearrangement. The additional double bond may be hydrogenated at a later stage.

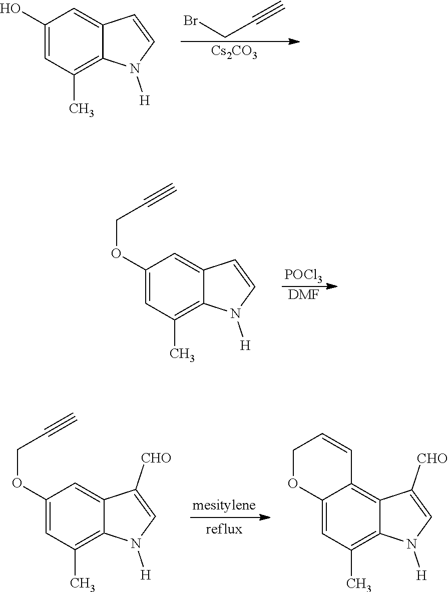

Compounds with linked 3- and 4-substituents are, for example, accessible through intramolecular Friedel-Crafts acylation.

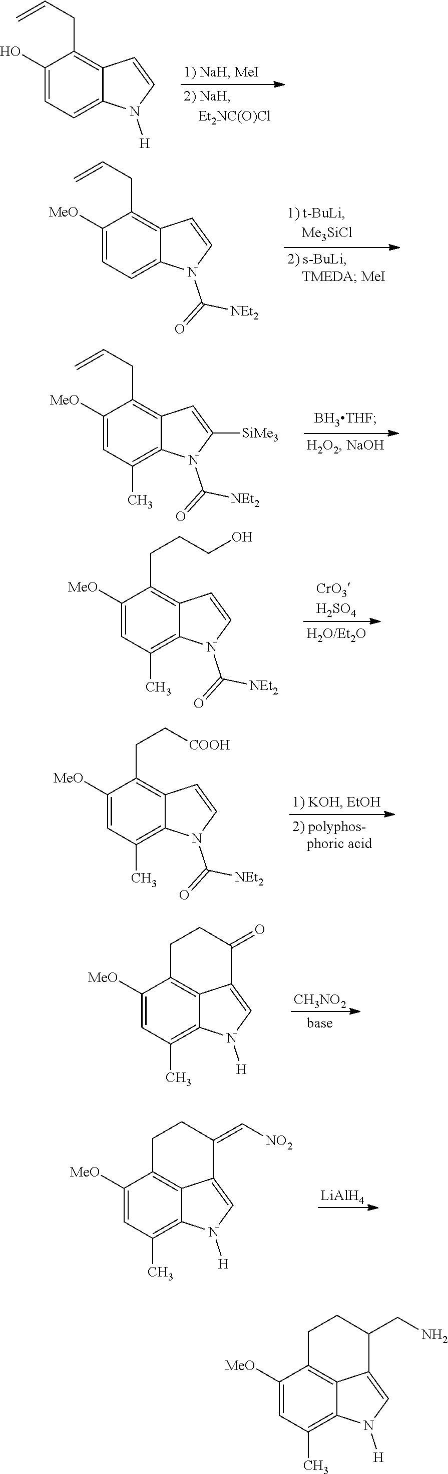

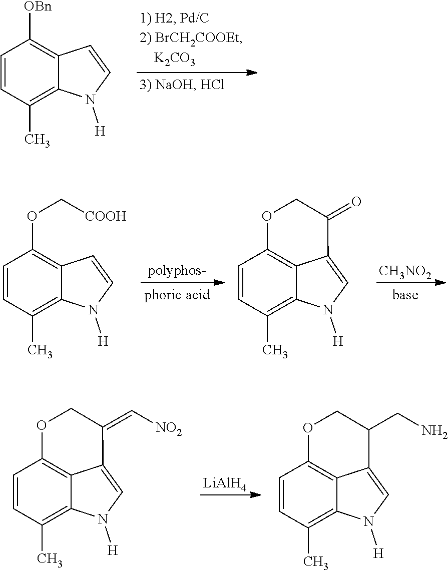

Deuterium may be incorporated into the compounds described herein in various ways, using deuterated versions of reagents and building blocks under the same or similar conditions as those employed for their counterparts with natural hydrogen isotope composition. The reduction of 3-acyl groups on the indole nucleus and of carboxamides with commercially available LiAlD₄ or BD₃-THF complex has already been mentioned. LiAlD₄ can be used in the same manner to reduce urethane functions, such as Boc- or Cbz-derivatized amines, to N-CD₃. The building blocks methyl-d₃ iodide, ethyl-d₅ iodide, allyl-d₅ bromide, formaldehyde-d₂ aqueous solution, paraformaldehyde-d₂, and dimethylamine-d₆ (free base and hydrochloride) are commercially available, as are the reducing agents commonly employed in reductive aminations/alkylations, NaBD₄ and NaBD₃CN. Deuterium gas is available for the catalytic deuteration and deuterolysis of CC multiple bonds and C-heteroatom bonds, respectively. Indole-d₇ is commercially available. Electron-rich aromatics, of which indoles are an example, can be ring-deuterated with D₂O in the presence of the catalyst, B(C₆F₅)₃, specifically in those positions that are more susceptible to electrophilic attack than an unactivated aromatic ring (Li, W.; Wang, M.-M.; Hu, Y.; Werner, T. Org. Lett. 2017, 19, 5768).

Aromatics and heteroaromatics may also be deuterated by reaction with an excess of D₂O in the presence of a heterogeneous transition metal catalyst (Sawama, Y.; Park, K.; Yamada, T.; Sajiki, H. Chem. Pharm. Bull. 2018, 66, 21-28). Deuteration of specific positions in the indole ring is achievable by halogen-metal exchange reactions on compounds that bear a halogen atom (typically Br or I) at the position to be deuterated, followed by quenching of the indolylmetal intermediate with a deuterating agent such as D₂O or CH₃OD; or by free-radical deuterodehalogenation of the same precursors with Bu₃SnD and a radical starter such as azobis(isobutyronitrile) or dibenzoyl peroxide; or by reaction of the same precursors with a deuteride source such as Bu₃SnD or formic acid-d₂ and a transition metal catalyst.

Compounds disclosed herein that possess a free phenolic hydroxyl group are more prone to air or enzymatic oxidation than the derived phenol ethers, and may be more polar than desirable for optimum brain penetration. To alleviate these and other concerns, the free phenolic hydroxyl groups may be protected by electron-withdrawing moieties, such as acyl, carbamyl, or phosphoryl derivatives, that are cleaved by hydrolytic enzymes to return the free phenols. Esterification of the phenolic hydroxyl can be accomplished with acyl halides or acid anhydrides, usually in the presence of a base, or with free carboxylic acids in the presence of a suitable activating agent with which the carboxylic acid undergoes an initial reaction to form a more electrophilic derivative. Urethanes (carbamyl derivatives) are obtained from phenols by reaction with isocyanates or N,N-dialkylcarbamyl halides in the presence of a base or other catalyst. Phosphoric acid derivatives suitable for phosphorylation include POCl₃ (Kargbo, R. B. et al. ACS Omega 2020, 5, 16959-16966), di“alkyl” chlorophosphates, and tetra“alkyl” diphosphates, where “alkyl” denotes hydrocarbon residues of various structures that are chosen in such a way as to be easily removable. A commonly used “alkyl” group for this purpose is benzyl. It has been observed that one of the benzyl groups of the resulting aryl dibenzyl phosphate tends to quaternize the side chain amine function, resulting in the formation of a zwitterion, which subsequently undergoes hydrogenolytic cleavage of both differential benzyl groups with hydrogen in the presence of a transition metal catalyst (Shirota, O. et al. J. Nat. Prod. 2003, 66, 885-887; Sherwood, A. M. et al. Synthesis 2020, 52, 688-694).

### Example A of Chemical Synthesis of Compound 1

### Compound 1: 3-[2-(Dimethylamino)ethyl]-7-methylindol-4-ol

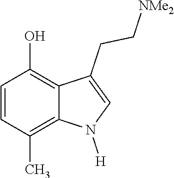 [SMILES: Cc1ccc(O)c2c(CCN(C)C)c[nH]c12]

An example of a synthesis of 3-[2-(Dimethylamino)ethyl]-7-methylindol-4-ol is provided as follows:

### 4-(Benzyloxy)-1-methyl-2-nitrobenzene

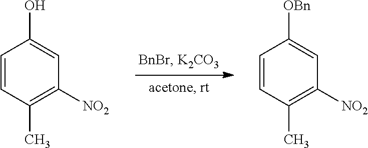 [SMILES: Cc1ccc(O)cc1[N+](=O)[O-] ; Cc1ccc(OCc2ccccc2)cc1[N+](=O)[O-]]

Benzyl bromide (333 g, 1.46 mol, 1.5 equiv.) was added to a stirred mixture comprising 4-methyl-3-nitrophenol (200 g, 1.31 mol), K₂CO₃ (541 g, 3.91 mol, 3.0 equiv.), and acetone (2.0 L). The resulting mixture was stirred at 20-25° C. for 16 hours and then filtered. The filtrate was concentrated under vacuum. The residue was combined with that from another batch (same scale), and the product was precipitated with hexane (100 mL) to yield 4-(benzyloxy)-1-methyl-2-nitrobenzene (470 g, 74%) as a light yellow solid. ¹H NMR (300 MHz, DMSO-d₆) δ 7.61 (d, J=2.7 Hz, 1H), 7.51-7.25 (m, 7H), 5.19 (s, 2H), 2.42 (s, 3H).

### 4-(Benzyloxy)-7-methylindole

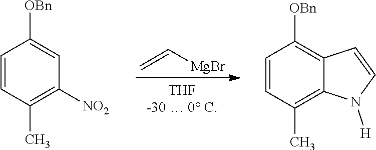

Vinylmagnesium bromide (1.0 M in THF, 1.64 L, 1.64 mol, 4.0 equiv.) was added dropwise to a stirred solution of 4-(benzyloxy)-1-methyl-2-nitrobenzene (100 g, 0.41 mol) in THF (2.0 L) at −30° C. under a N₂ atmosphere. The resulting mixture was stirred at 0° C. for 2 hours and then quenched with a NH₄Cl solution. The organic layer was separated and concentrated under vacuum. The residue was combined with those from another three batches (same scale) and purified by flash chromatography on silica gel (0-20% EtOAc in petroleum ether) to yield 4-(benzyloxy)-7-methylindole (134 g, 34%) as a yellow oil. MS (ESI, m/z): 238 (M+H)⁺.

### 2-[4-(Benzyloxy)-7-methylindol-3-yl]-N,N-dimethylglyoxylamide

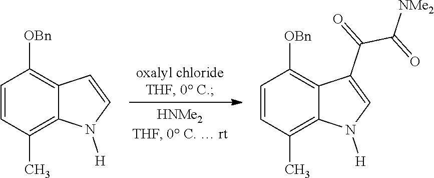 [SMILES: Cc1ccc(OCc2ccccc2)c2cc[nH]c12 ; Cc1ccc(OCc2ccccc2)c2c(C(=O)C(=O)N(C)C)c[nH]c12]

Oxalyl chloride (64.3 g, 0.51 mol, 3.0 equiv.) was added dropwise to a stirred solution of 4-(benzyloxy)-7-methylindole (40.0 g, 0.17 mol) in THF (800 mL) at 0° C. under a N₂ atmosphere. The resulting mixture was stirred at 0° C. for 2 h. A solution of dimethylamine in THF (2.0 M, 0.51 L, 1.02 mol, 6.0 equiv.) was added dropwise. The resulting mixture was stirred at 20-25° C. for an additional 1 hour and then extracted three times with EtOAc. The combined organic layers were washed three times with brine, dried over Na₂SO₄, and concentrated under vacuum. The residue was combined with those from another two batches (same scale) and purified by flash chromatography on silica gel (0-100% EtOAc containing 0.05% triethylamine in petroleum ether containing 0.05% triethylamine) to yield 2-[4-(benzyloxy)-7-methyl-1H-indol-3-yl]-N,N-dimethylglyoxylamide (90.0 g, 52%) as a brown solid. MS (ESI, m/z): 337 (M+H)⁺.

### [2-[4-(Benzyloxy)-7-methylindol-3-yl]ethyl]dimethylamine

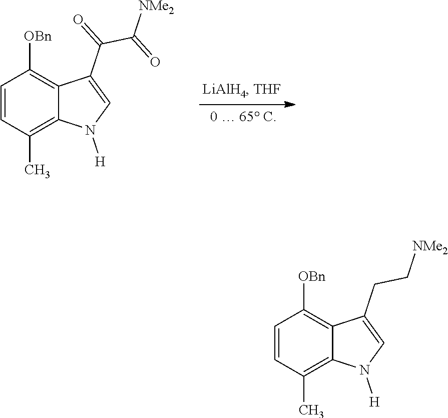 [SMILES: Cc1ccc(OCc2ccccc2)c2c(C(=O)C(=O)N(C)C)c[nH]c12 ; Cc1ccc(OCc2ccccc2)c2c(CCN(C)C)c[nH]c12]

Lithium aluminum hydride (22.6 g, 595 mmol, 10 equiv.) was added in portions to a stirred solution of 2-[4-(benzyloxy)-7-methylindol-3-yl]-N,N-dimethylglyoxylamide (20.0 g, 59.5 mmol) in THE (400 mL) at 0° C. under a N₂ atmosphere. The mixture was stirred at 65° C. for 16 hours, and then quenched by addition of Na₂SO₄·10H₂O (230 g, 714 mmol, 12 equiv.) at 0° C. in portions until bubbling ceased, and filtered. The filtrate was concentrated under vacuum. The residue was combined with those from another three batches (same scale) and purified by flash chromatography on silica gel (0-20% methanol containing 0.05% triethylamine in CH₂Cl₂ containing 0.05% triethylamine) to afford [2-[4-(benzyloxy)-7-methylindol-3-yl]ethyl]dimethylamine (40.0 g, 54%) as a brown solid. MS (ESI, m/z): 309 (M+H)⁺.

### 3-[2-(Dimethylamino)ethyl]-7-methylindol-4-ol

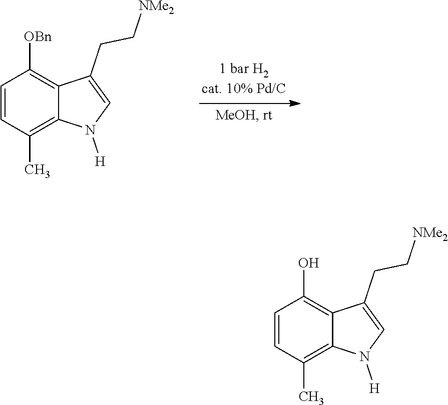 [SMILES: Cc1ccc(OCc2ccccc2)c2c(CCN(C)C)c[nH]c12 ; Cc1ccc(O)c2c(CCN(C)C)c[nH]c12]

10% Pd/C (wet, 750 mg) was added to a stirred solution of [2-[4-(benzyloxy)-7-methylindol-3-yl]ethyl]dimethylamine (5.0 g, 16.2 mmol) in methanol (50 mL) at ambient temperature, and the reaction mixture was placed under a H₂ atmosphere. The mixture was stirred at 20-25° C. for 1.5 hours and then filtered. The filtrate was concentrated under vacuum. The residue, combined with those from another six batches (same scale), was purified by reverse phase flash chromatography on C₁₈ silica gel (5-20% acetonitrile in water) and further purified by supercritical fluid chromatography to yield 3-[2-(dimethylamino)ethyl]-7-methylindol-4-ol (5.56 g, 22%) as a light yellow solid. ¹H NMR (400 MHz, DMSO-d₆) δ 10.49 (br s, 1H), 10.42 (br s, 1H), 6.92 (d, J=2.4 Hz, 1H), 6.58 (d, J=7.6 Hz, 1H), 6.18 (d, J=7.6 Hz, 1H), 2.89 (t, J=6.8 Hz, 2H), 2.54 (t, J=6.8 Hz, 2H), 2.29 (s, 3H), 2.21 (s, 6H). MS (ESI, m/z): 219 (M+H)⁺.

### Example B of Chemical Synthesis of Compound 1

Another example of a synthesis of 3-[2-(dimethylamino)ethyl]-7-methylindol-4-ol is provided as follows:

### 4-Methoxy-7-methylindole

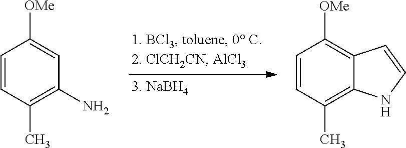

To a solution of 5-methoxy-2-methylaniline (50 g, 0.364 mol) in anhydrous CH₂Cl₂ (500 mL) was added with ice cooling and exclusion of moisture a solution of BCl₃ (1M in CH₂Cl₂, 400 mL, 1.1 equiv.). Chloroacetonitrile (48 mL, 0.73 mol, 2.0 equiv.) was then added, followed by AlCl₃ (53.3 g, 0.40 mol, 1.1 equiv.). The resulting mixture was stirred overnight at rt. Aqueous NH₄Cl solution was added, and the mixture was filtered. The filter cake was washed with CH₂Cl₂, and the phases of the filtrate were separated and the aqueous phase further extracted with CH₂Cl₂. The combined organic phases were washed with brine, dried over Na₂SO₄, and concentrated under reduced pressure. The residue was dissolved in a mixture of dioxane and H₂O (10:1), then NaBH₄ (69 g) was added. The mixture was stirred overnight at room temperature. Water was added, and the mixture was filtered. The filtrate was extracted with CH₂Cl₂. The combined organic phases were washed with brine, dried over Na₂SO₄, and concentrated under reduced pressure. The residue was purified by column chromatography on silica gel (eluent: EtOAc/petroleum ether 1:1) to afford the indole as an off-white solid (20 g, 34%). MS (ESI) calcd for C₁₀H₁₁NO: 161. found: 162 (M+H⁺). ¹H NMR (400 MHz, CDCl₃) δ 8.06 (br s, 1H), 7.12 (t, J=2.8 Hz, 1H), 6.89 (d, J=7.6 Hz, 1H), 6.67 (t, J=2.8 Hz, 1H), 6.45 (d, J=7.6 Hz, 1H), 3.94 (s, 3H), 2.43 (s, 3H).

### 2-(4-Methoxy-7-methylindol-3-yl)-N,N-dimethylglyoxylamide

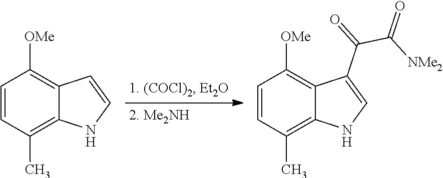 [SMILES: COc1ccc(C)c2[nH]ccc12 ; COc1ccc(C)c2[nH]cc(C(=O)C(=O)N(C)C)c12]

To a solution of 4-methoxy-7-methylindole (10.0 g, 62.1 mmol) in anhydrous diethyl ether (200 mL) was added dropwise with ice-salt cooling oxalyl chloride (15.6 g, 124 mmol, 2 equiv.). The mixture was stirred for 3 h in the cold bath, after which time period the indole was found to have been consumed. The resulting slurry was added dropwise with ice-salt cooling to a 40% aqueous solution of Me₂NH (56 mL). The resulting brown slurry was stirred for 1 h at 0° C., then warmed to room temperature and stirred overnight. The reaction mixture was filtered, and the filtrate was treated with aq. NaHCO₃, then extracted with CH₂Cl₂. The combined organic phases were washed with water and brine, dried with Na₂SO₄, and concentrated under reduced pressure. The residue was purified by column chromatography on silica gel (eluent: CH₂Cl₂/MeOH 60:1) to afford 2-(4-methoxy-7-methylindol-3-yl)-N,N-dimethylglyoxylamide as a brown solid (12.2 g, 75%). MS (ESI) calcd for C₁₄H₁₆N₂O₃: 260. found: 261 (M+H⁺). ¹H NMR (400 MHz, CDCl₃) δ 9.86 (br s, 1H), 7.63 (d, J=3.2 Hz, 1H), 6.87 (d, J=8.0 Hz, 1H), 6.52 (d, J=8.0 Hz, 1H), 3.88 (s, 3H), 3.13 (s, 3H), 3.08 (s, 3H), 2.32 (s, 3H).

### [2-(4-Methoxy-7-methylindol-3-yl)ethyl]dimethylamine

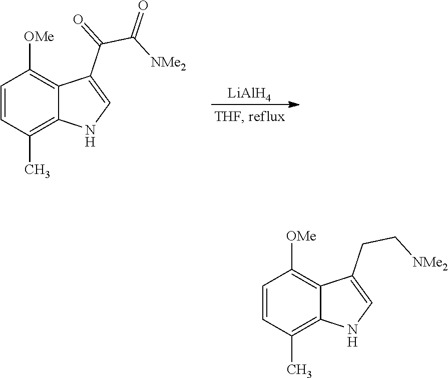 [SMILES: COc1ccc(C)c2[nH]cc(C(=O)C(=O)N(C)C)c12 ; "LiAlH4" ; COc1ccc(C)c2[nH]cc(CCN(C)C)c12]

To a slurry of LiAlH₄ (6.66 g, 176 mmol) in anhydrous THF (120 mL) was added with ice-salt cooling under a nitrogen atmosphere a slurry of 2-(4-methoxy-7-methylindol-3-yl)-N,N-dimethylglyoxylamide (12.2 g, 47 mmol) in anhydrous THF (180 mL). The mixture was heated to reflux overnight. A solution of 20 vol % water in THF was then added dropwise with ice cooling. The mixture was stirred for 0.5 h, then filtered, and the filter cake was washed with THF. The phases of the filtrate were separated and the aqueous phase further extracted with EtOAc. The combined organic phases were washed with brine, dried over NaSO₄, and concentrated under reduced pressure. The residue was purified by column chromatography on silica gel (eluent: CH₂Cl₂/3.5M NH₃ in MeOH 30:1) to afford the amine as a brown solid (8.2 g, 75%). MS (ESI) calcd for C₁₄H₂₀N₂O: 232. found: 233 (M+H⁺). ¹H NMR (400 MHz, CDCl₃) δ 7.87 (br s, 1H), 6.90 (s, 1H), 6.85 (d, J=8 Hz, 1H), 6.40 (d, J=7.6 Hz, 1H), 3.89 (s, 3H), 3.07-3.03 (m, 2H), 2.64-2.60 (m, 2H), 2.38 (s, 3H), 2.34 (s, 6H).

### 3-[2-(Dimethylamino)ethyl]-7-methylindol-4-ol

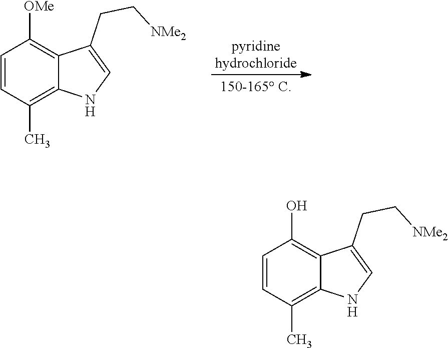 [SMILES: COc1ccc(C)c2[nH]cc(CCN(C)C)c12 ; "pyridinehydrochloride" ; Cc1ccc(O)c2c(CCN(C)C)c[nH]c12]

A mixture of [2-(4-methoxy-7-methylindol-3-yl)ethyl]dimethylamine (2.4 g, 10.3 mmol) and pyridine hydrochloride (24.0 g, 206 mmol, 20 equiv.) was placed in a 100 mL round-bottom flask and heated at 150-165° C. for 4.5 h. The reaction mixture was cooled to room temperature and dissolved in water, basified with Na₂CO₃, and extracted with EtOAc. The organic phase was washed with brine, dried over NaSO₄, and concentrated under reduced pressure. The residue was purified by column chromatography on silica gel (eluent: CH₂Cl₂/3.5M NH₃ in MeOH 40:1) to afford Compound 1 as an off-white solid (1.7 g, 75%). MS (ESI) calcd for C₁₃H₁₈N₂O: 218. found: 219 (M+H⁺). ¹H NMR (400 MHz, CDCl₃) δ 13.23 (br s, 1H), 7.80 (s, 1H), 6.86 (s, 1H), 6.85 (d, J=8.4 Hz, 1H), 6.50 (d, J=7.6 Hz, 1H), 2.95-2.93 (m, 2H), 2.70-2.68 (m, 2H), 2.37 (s, 9H).

### Example of Chemical Synthesis of Compound 7

### Compound 7: 3-[2-(Dimethylamino)ethyl]-7-(2-hydroxyethyl)indol-4-ol

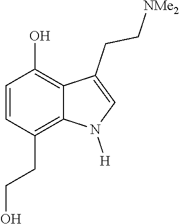 [SMILES: CN(C)CCc1c[nH]c2c(CCO)ccc(O)c12]

An example of a synthesis of 3-[2-(dimethylamino)ethyl]-7-(2-hydroxyethyl)indol-4-ol is provided as follows:

### 4-(Benzyloxy)-1-chloro-2-nitrobenzene

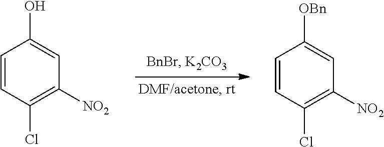 [SMILES: O=[N+]([O-])c1cc(O)ccc1Cl ; O=[N+]([O-])c1cc(OCc2ccccc2)ccc1Cl]

To a solution of 4-chloro-3-nitrophenol (22.0 g, 126 mmol) in acetone (210 mL) and DMF (210 mL) were added K₂CO₃ (27 g, 196 mmol) and benzyl chloride (20 g, 156 mmol) under nitrogen atmosphere. The mixture was stirred overnight at 20-25° C. Water was added, and the resulting mixture was extracted with CH₂Cl₂. The phases were separated, and the organic phase was washed with water and brine, dried over Na₂SO₄, and concentrated under reduced pressure. The residue was purified by column chromatography on silica gel (eluent: EtOAc/petroleum ether 1:20) to afford 4-(benzyloxy)-1-chloro-2-nitrobenzene as a light yellow oil (27.0 g, 81%).

### Diethyl 2-[4-(Benzyloxy)-2-nitrophenyl]malonate

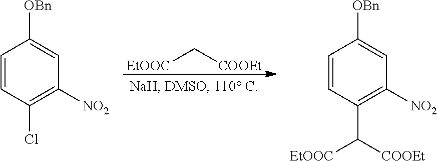 [SMILES: O=[N+]([O-])c1cc(OCc2ccccc2)ccc1Cl ; CCOC(=O)CC(=O)OCC ; CCOC(=O)C(C(=O)OCC)c1ccc(OCc2ccccc2)cc1[N+](=O)[O-]]

To a suspension of NaH (10.6 g, 267 mmol) in DMSO (345 mL) was added dropwise with ice cooling a solution of diethyl malonate (44.5 g, 278 mmol) in DMSO (20 mL), followed by a solution of 4-(benzyloxy)-1-chloro-2-nitrobenzene (27.0 g, 102 mmol) in DMSO (40 mL). The mixture was stirred overnight at 110° C. The resulting dark brown suspension was quenched with acetic acid and diluted with CH₂Cl₂ and 0.5M aqueous HCl. The phases were separated, and the mixture was further extracted with CH₂Cl₂. The combined organic phases were washed with water and brine, dried over Na₂SO₄, and concentrated under reduced pressure. The residue was purified by column chromatography on silica gel (eluent: EtOAc/petroleum ether 1:30) to afford diethyl 2-[4-(benzyloxy)-2-nitrophenyl]malonate as a yellow oil (24.5 g, 62%). ¹H NMR (400 MHz, CDCl₃) δ 7.65 (d, J=2.4 Hz, 1H), 7.44-7.36 (m, 6H), 7.23 (dd, J=2.8 and 8.8 Hz, 1H), 5.22 (s, 1H), 5.13 (s, 2H), 4.26-4.18 (m, 4H), 1.30-1.26 (m, 6H).

### 2-[4-(Benzyloxy)-2-nitrophenyl]acetic acid

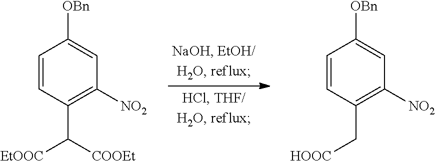 [SMILES: CCOC(=O)C(C(=O)OCC)c1ccc(OCc2ccccc2)cc1[N+](=O)[O-] ; O=C(O)Cc1ccc(OCc2ccccc2)cc1[N+](=O)[O-]]

To a solution of diethyl 2-(4-(benzyloxy)-2-nitrophenyl)malonate (20.0 g, 51.6 mmol) in EtOH (450 mL) was added 10% aqueous NaOH (600 mL) at 20-25° C. The resulting mixture was refluxed for 1.5 h. EtOH was removed under reduced pressure, and THF was added with ice cooling. Aqueous HCl (6M) was added dropwise to pH 1, then the solution was refluxed for 1 h. THF was removed, and the residue was extracted with CH₂Cl₂. The phases were separated, and the organic phase was dried over Na₂SO₄ and concentrated under reduced pressure. The residue was purified by column chromatography on silica gel (eluent: CH₂Cl₂/MeOH 10:1) to afford 2-[4-(benzyloxy)-2-nitrophenyl]acetic acid as a yellow solid (13.1 g, 88%).

### 2-[4-(Benzyloxy)-2-nitrophenyl]ethanol

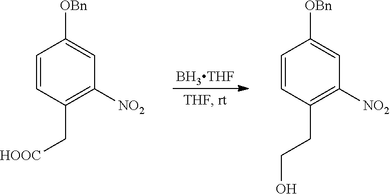 [SMILES: O=C(O)Cc1ccc(OCc2ccccc2)cc1[N+](=O)[O-] ; O=[N+]([O-])c1cc(OCc2ccccc2)ccc1CCO ; "BH3·THF"]

To a solution of 2-[4-(benzyloxy)-2-nitrophenyl]acetic acid (15.5 g, 54 mmol) in THF (150 mL) was added dropwise with ice cooling BH₃·THF solution (1M in THF; 135 mL, 2.5 equiv.). The mixture was stirred for 5 h at 20-25° C. The mixture was quenched with H₂O and extracted with EtOAc. The phases were separated, and the organic phase was dried over Na₂SO₄ and concentrated under reduced pressure. The residue was purified by column chromatography on silica gel (eluent: EtOAc/petroleum ether 1:5) to afford the title product as a brown solid (8.0 g, 54%). ¹H NMR (400 MHz, CDCl₃) δ 7.54 (d, J=2 Hz, 1H), 7.44-7.33 (m, 5H), 7.31 (d, J=8.8 Hz, 1H), 7.18 (dd, J=2.8 and 8.4 Hz, 1H), 5.11 (s, 2H), 3.91 (t, J=6.4 Hz, 2H), 3.10 (t, J=6.4 Hz, 2H).

### 4-(Benzyloxy)-1-[2-(methoxymethoxy)ethyl]-2-nitrobenzene

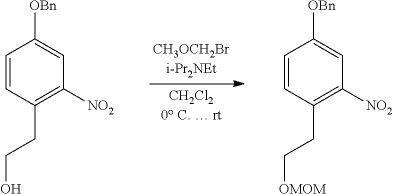

To a solution of 2-[4-(benzyloxy)-2-nitrophenyl]ethanol (2.0 g, 7.3 mmol) in CH₂Cl₂ (40 mL) were added with ice cooling ethyldiisopropylamine (2.84 g, 21.9 mmol) and bromomethyl methyl ether (1.83 g, 14.6 mmol). The mixture was warmed to 20-25° C. and was stirred at this temperature overnight. The reaction was quenched with MeOH with ice cooling and diluted with CH₂Cl₂. The solution was washed with brine. The organic phase was dried over Na₂SO₄ and concentrated under reduced pressure. The residue was purified by column chromatography on silica gel (eluent: EtOAc/petroleum ether 1:8) to afford 4-(benzyloxy)-1-[2-(methoxymethoxy)ethyl]-2-nitrobenzene as a yellow oil (2.0 g, 86%). ¹H NMR (400 MHz, CDCl₃) δ 7.53 (d, J=2.8 Hz, 1H), 7.44-7.34 (m, 5H), 7.31 (d, J=8.8 Hz, 1H), 7.14 (dd, J=2.8 and 8.4 Hz, 1H), 5.10 (s, 2H), 4.58 (s, 2H), 3.77 (t, J=6.4 Hz, 2H), 3.27 (s, 3H), 3.14 (t, J=6.4 Hz, 2H).

### 4-(Benzyloxy)-7-[2-(methoxymethoxy)ethyl]indole

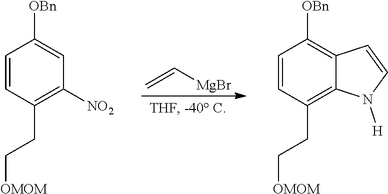

To a solution of 4-(benzyloxy)-1-(2-[methoxymethoxy)ethyl]-2-nitrobenzene (1.00 g, 3.15 mmol) in THF (16 mL) was added dropwise at −40° C. vinylmagnesium bromide solution (1.0M in THF; 11.0 mL). The resulting mixture was stirred for 4 h at −40° C. The reaction was quenched with saturated aqueous NH₄Cl solution and extracted with EtOAc. The phases were separated, and the organic phase was dried over Na₂SO₄ and concentrated under reduced pressure. The residue was purified by column chromatography on silica gel (eluent: EtOAc/petroleum ether 1:8) to afford 4-(benzyloxy)-7-[2-(methoxymethoxy)ethyl]indole as a yellow oil (385 mg, 40%). ¹H NMR (400 MHz, CDCl₃) δ 9.05 (br s, 1H), 7.50 (d, J=7.6 Hz, 2H), 7.39 (t, J=7.2 Hz, 2H), 7.33-7.30 (m, 1H), 7.14-7.13 (m, 1H), 6.86 (d, J=8 Hz, 1H), 6.71-6.70 (m, 1H), 6.50 (d, J=8 Hz, 1H), 5.22 (s, 2H), 4.66 (s, 2H), 3.86 (t, J=6 Hz, 2H), 3.28 (s, 3H), 3.08 (t, J=6 Hz, 2H).

### 2-[4-(Benzyloxy)-7-[2-(methoxymethoxy)ethyl]indol-3-yl]-N,N-dimethylglyoxylamide

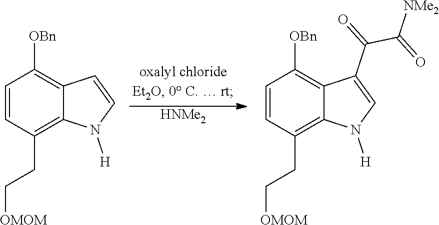

To a solution of 4-(benzyloxy)-7-[2-(methoxymethoxy)ethyl]indole (385 mg, 1.24 mmol) in diethyl ether (7 mL) was added dropwise with ice cooling oxalyl chloride (314 mg, 2.47 mmol). The mixture was stirred for 3 h and then added dropwise with ice/salt cooling into 40% aqueous Me₂NH solution (5 mL). The mixture was warmed to 20-25° C. and stirred at this temperature overnight, then was washed with water and brine. The phases were separated, and the organic phase was dried over Na₂SO₄, and concentrated under reduced pressure. The residue was purified by column chromatography on silica gel (eluent: EtOAc/petroleum ether 1:1) to afford 2-[4-(benzyloxy)-7-[2-(methoxymethoxy)ethyl]indol-3-yl]-N,N-dimethylglyoxylamide as a yellow oil (444 mg, 87%). ¹H NMR (400 MHz, CDCl₃) δ 10.09 (br s, 1H), 8.00 (d, J=3.2 Hz, 1H), 7.55-7.52 (m, 2H), 7.42-7.35 (m, 2H), 7.32-7.29 (m, 1H), 6.90 (d, J=8 Hz, 1H), 6.58 (d, J=8 Hz, 1H), 5.26 (s, 2H), 4.67 (s, 2H), 3.84 (t, J=6 Hz, 2H), 3.29 (s, 3H), 3.06 (t, J=5.2 Hz, 2H), 3.00 (s, 3H), 2.93 (s, 3H).

### [2-[4-(Benzyloxy)-7-[2-(methoxymethoxy)ethyl]indol-3-yl]ethyl]dimethylamine

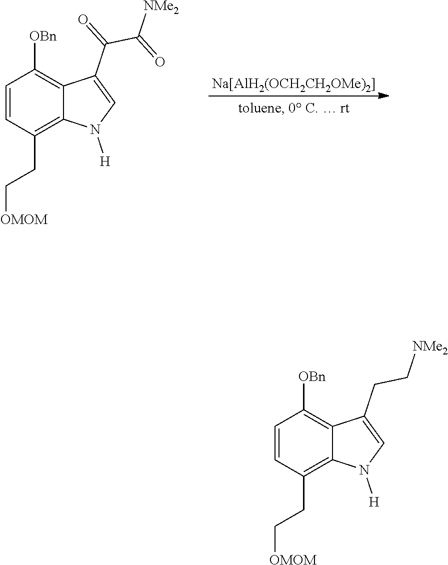

To a solution of 2-[4-(benzyloxy)-7-[2-(methoxymethoxy)ethyl]indol-3-yl]-N,N-dimethylglyoxylamide (440 mg, 1.07 mmol) in toluene (6 mL) was added dropwise with ice cooling sodium bis(2-methoxyethoxy)aluminum dihydride (70% in toluene, 2.5 g, 8.5 mmol, 8 equiv.). The mixture was warmed to 20-25° C. and stirred at this temperature overnight. The reaction was quenched with water, and 15% aqueous NaOH solution was added. The mixture was extracted with EtOAc. The organic phase was washed with water and brine, dried over Na₂SO₄, and concentrated under reduced pressure. The residue was purified by column chromatography on silica gel (eluent: CH₂Cl₂/3.5M NH₃ in MeOH 50:1) to afford [2-[4-(benzyloxy)-7-[2-(methoxymethoxy)ethyl]indol-3-yl]ethyl]dimethylamine as a light yellow solid (205 mg, 50%). MS (ESI, m/z): 383 (M+H)⁺. ¹H NMR (400 MHz, CDCl₃) δ 8.79 (br s, 1H), 7.49 (d, J=7.2 Hz, 2H), 7.38 (t, J=7.2 Hz, 2H), 7.32 (d, J=7.2 Hz, 1H), 6.91 (d, J=2.4 Hz, 1H), 6.83 (d, J=8 Hz, 1H), 6.46 (d, J=8 Hz, 1H), 5.17 (s, 2H), 4.65 (s, 2H), 3.84 (t, J=6 Hz, 2H), 3.30 (s, 3H), 3.07-3.03 (m, 4H), 2.62-2.58 (m, 2H), 2.14 (s, 6H).

### 3-[2-(Dimethylamino)ethyl]-7-[2-(methoxymethoxy)ethyl]indol-4-ol

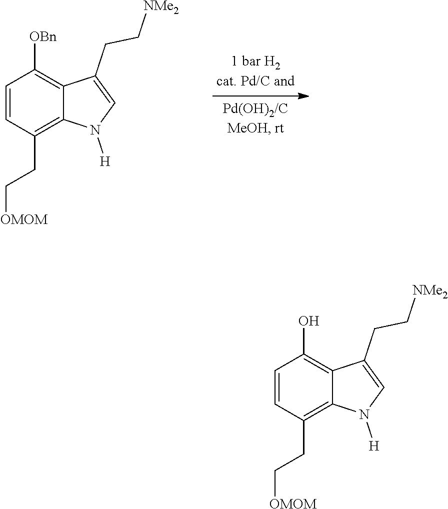

To a solution of [2-[4-(benzyloxy)-7-[2-(methoxymethoxy)ethyl]indol-3-yl]ethyl]dimethylamine (175 mg, 0.45 mmol) in MeOH (5 mL) were added 10% Pd/C (20 mg) and 20% Pd(OH)₂/C (20 mg). The mixture was stirred under a hydrogen atmosphere for 2 h at 20-25° C., then filtered. The filtrate was concentrated. The residue was purified by column chromatography on silica gel (eluent: CH₂Cl₂/3.5M NH₃ in MeOH 50:1) to afford 3-[2-(dimethylamino)ethyl]-7-[2-(methoxymethoxy)ethyl]indol-4-ol as a light yellow solid (109 mg, 82%).

### 3-[2-(Dimethylamino)ethyl]-7-[2-hydroxyethyl]indol-4-ol

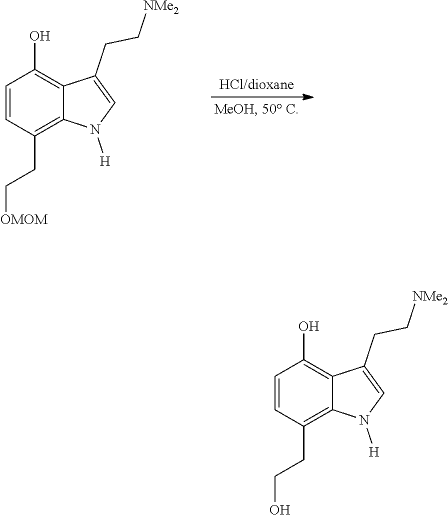

To a solution of 3-[2-(dimethylamino)ethyl]-7-[2-(methoxymethoxy)ethyl]indol-4-ol (87 mg, 0.3 mmol) in MeOH (3 mL) was added a solution of anhydrous HCl (4.0M in dioxane; 0.3 mL, 1.2 mmol, 4 equiv.). The mixture was stirred at 50° C. for 3 h, cooled to 20-25° C., basified with aqueous NaHCO₃ solution, and extracted with EtOAc. The organic phase was dried over Na₂SO₄ and concentrated under reduced pressure. The residue was purified by column chromatography on silica gel (eluent: CH₂Cl₂/3.5M NH₃ in MeOH 50:1) to afford 3-[2-(dimethylamino)ethyl]-7-[2-hydroxyethyl]indol-4-ol as a light brown solid (46 mg, 64%). MS (ESI, m/z): 249 (M+H)⁺. ¹H NMR (400 MHz, CDCl₃) δ 8.64 (br s, 1H), 6.85 (s, 1H), 6.84 (d, J=8 Hz, 1H), 6.50 (d, J=8 Hz, 1H), 3.95 (t, J=6 Hz, 2H), 2.99 (t, J=6 Hz, 2H), 2.96-2.93 (m, 2H), 2.71-2.69 (m, 2H), 2.38 (s, 6H).

### Example of Chemical Synthesis of Compound 8

### Compound 8: 3-[2-(Dimethylamino)ethyl]-7-(2-fluoroethyl)indol-4-ol

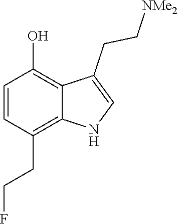 [SMILES: CN(C)CCc1c[nH]c2c(CCF)ccc(O)c12]

An example of a synthesis of 3-[2-(dimethylamino)ethyl]-7-(2-fluoroethyl)indol-4-ol is provided as follows:

### 4-(Benzyloxy)-1-(2-fluoroethyl)-2-nitrobenzene

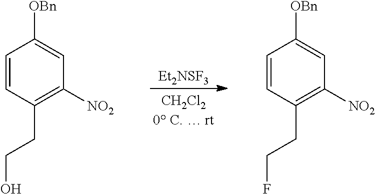 [SMILES: "Et2NSF3" ; O=[N+]([O-])c1cc(OCc2ccccc2)ccc1CCO ; O=[N+]([O-])c1cc(OCc2ccccc2)ccc1CCF]

To a solution of 2-[4-(benzyloxy)-2-nitrophenyl]ethanol (6.4 g, 22 mmol) in CH₂Cl₂ (100 mL) was added with ice cooling (diethylamino)sulfur trifluoride (DAST; 7.1 g, 44 mmol, 2 equiv.). The mixture was warmed to 20-25° C. and stirred for 5 h. The reaction was quenched with ice cooling with aqueous NaHCO₃ solution and extracted with CH₂Cl₂. The organic phase was dried over Na₂SO₄ and concentrated under reduced pressure. The residue was purified by column chromatography on silica gel (eluent: EtOAc/petroleum ether 1:20) to afford 4-(benzyloxy)-1-(2-fluoroethyl)-2-nitrobenzene as a brown oil (2.1 g, 32%). ¹H NMR (400 MHz, CDCl₃) δ 7.59 (d, J=2.4 Hz, 1H), 7.44-7.34 (m, 5H), 7.32 (d, J=8 Hz, 1H), 7.17 (dd, J=2.4 Hz and 8 Hz, 1H), 5.11 (s, 2H), 4.69 (dt, J=6 Hz (t) and 48 Hz (d), 2H), 3.24 (dt, J=6 Hz (t) and 24 Hz (d), 2H).

### 4-(Benzyloxy)-7-(2-fluoroethyl)indole

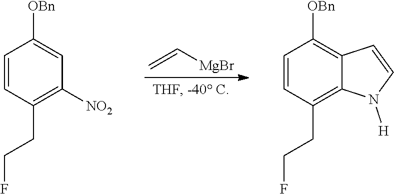

To a solution of 4-(benzyloxy)-1-(2-fluoroethyl)-2-nitrobenzene (2.1 g, 7.6 mmol) in THF (26 mL) was added dropwise at −40° C. vinylmagnesium bromide solution (1.0M in THF, 26 mL, 3.4 equiv.). The mixture was stirred for 4 h at −40° C. The reaction was quenched with saturated aqueous NH₄Cl solution and extracted with EtOAc. The organic phase was dried over Na₂SO₄ and concentrated under reduced pressure. The residue was purified by column chromatography on silica gel (eluent: EtOAc/petroleum ether 1:20) to afford 4-(benzyloxy)-7-(2-fluoroethyl)indole as a brown solid (728 mg, 35%). ¹H NMR (400 MHz, CDCl₃) δ 8.45 (br s, 1H), 7.50 (d, J=7.2 Hz, 2H), 7.39 (t, J=7.2 Hz, 2H), 7.32 (t, J=7.2 Hz, 1H), 7.15 (t, J=2.8 Hz, 1H), 6.88 (d, J=8 Hz, 1H), 6.74 (t, J=2.4 Hz, 1H), 6.53 (d, J=8 Hz, 1H), 5.22 (s, 2H), 4.75 (dt, J=6 Hz (t) and 48 Hz (d), 2H), 3.18 (dt, J=6 Hz (t) and 28 Hz (d), 2H).

### 2-[4-(Benzyloxy)-7-(2-fluoroethyl)indol-3-yl]-N,N-dimethylglyoxylamide

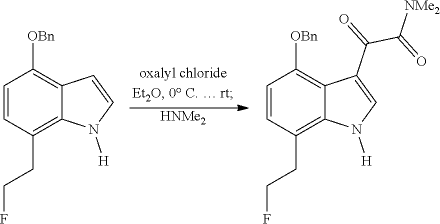 [SMILES: FCCc1ccc(OCc2ccccc2)c2cc[nH]c12 ; CN(C)C(=O)C(=O)c1c[nH]c2c(CCF)ccc(OCc3ccccc3)c12 ; "HNMe2"]

To a solution of 4-(benzyloxy)-7-(2-fluoroethyl)indole (425 mg, 1.94 mmol) in diethyl ether (15 mL) was added dropwise with ice cooling oxalyl chloride (400 mg, 3.98 mmol, 2 equiv.). The mixture was stirred for 3 h and then added dropwise with ice/salt cooling into 40% aqueous Me₂NH solution (5 mL). The mixture was warmed to 20-25° C. and stirred overnight, then washed with water and brine. The organic phase was dried over Na₂SO₄, and concentrated under reduced pressure. The residue was purified by column chromatography on silica gel (eluent: EtOAc/petroleum ether 1:3) to afford 2-[4-(benzyloxy)-7-(2-fluoroethyl)indol-3-yl]-N,N-dimethylglyoxylamide as a brown solid (540 mg, 76%). ¹H NMR (400 MHz, CDCl₃) δ 9.81 (br s, 1H), 7.86 (d, J=3.2 Hz, 1H), 7.52 (d, J=7.2 Hz, 2H), 7.37 (t, J=7.2 Hz, 2H), 7.30 (d, J=7.2 Hz, 1H), 6.89 (d, J=8 Hz, 1H), 6.58 (d, J=8 Hz, 1H), 5.24 (s, 2H), 4.66 (dt, J=6 Hz (t) and 47.2 Hz (d), 2H), 3.11 (dt, J=6 Hz (t) and 28.4 Hz (d), 2H), 2.97 (s, 3H), 2.91 (s, 3H).

### [2-[4-(Benzyloxy)-7-(2-fluoroethyl)indol-3-yl]ethyl]dimethylamine

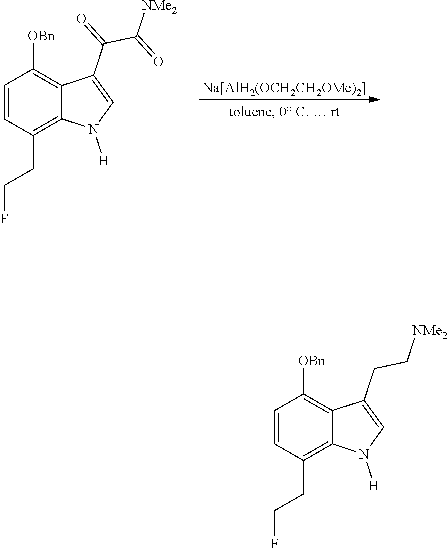

To a solution of 2-[4-(benzyloxy)-7-(2-fluoroethyl)indol-3-yl]-N,N-dimethylglyoxylamide (270 mg, 0.73 mmol) in toluene (7 mL) was added dropwise with ice cooling sodium bis(2-methoxyethoxy)aluminum dihydride solution (70% in toluene, Red-AI, 1.06 g, 3.67 mmol, 5 equiv.). The mixture was warmed to 20-25° C. and stirred overnight. The reaction was quenched with water, and 15% aqueous NaOH solution was added. The mixture was extracted with EtOAc, and the organic phase was washed with water and brine, dried over Na₂SO₄, and concentrated under reduced pressure. The residue was purified by column chromatography on silica gel (eluent: CH₂Cl₂/3.5M NH₃ in MeOH 30:1) to afford [2-[4-(benzyloxy)-7-(2-fluoroethyl)indol-3-yl]ethyl]dimethylamine as a brown solid (165 mg, 67%). MS (ESI, m/z): 341 (M+H)⁺. ¹H NMR (400 MHz, CDCl₃) δ 8.25 (br s, 1H), 7.49 (d, J=7.2 Hz, 2H), 7.40-7.30 (m, 3H), 6.92 (d, J=1.6 Hz, 1H), 6.85 (d, J=8 Hz, 1H), 6.49 (d, J=8 Hz, 1H), 5.17 (s, 2H), 4.72 (dt, J=6 Hz (t) and 47.6 Hz (d), 2H), 3.14 (dt, J=6 Hz (t) and 27.6 Hz (d), 2H), 3.08-3.04 (m, 2H), 2.64-2.60 (m, 2H), 2.15 (s, 6H).

### 3-[2-(Dimethylamino)ethyl]-7-(2-fluoroethyl)indol-4-ol

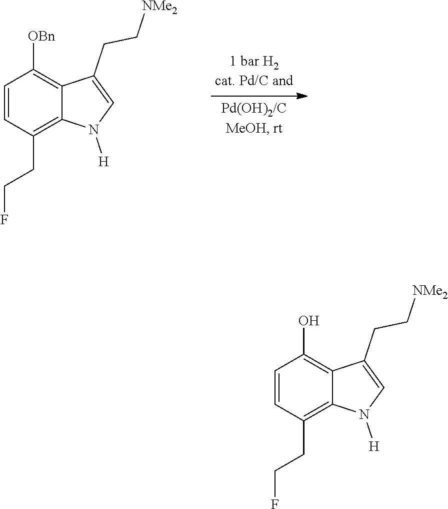 [SMILES: CN(C)CCc1c[nH]c2c(CCF)ccc(OCc3ccccc3)c12 ; CN(C)CCc1c[nH]c2c(CCF)ccc(O)c12]

To a solution of [2-[4-(benzyloxy)-7-(2-fluoroethyl)indol-3-yl]ethyl]dimethylamine (160 mg, 0.48 mmol) in MeOH (8 mL) was added 10% Pd/C (20 mg) and 20% Pd(OH)₂/C (20 mg). The mixture was stirred for 2 h at 20-25° C. under a hydrogen atmosphere, then filtered, and the filtrate was concentrated. The residue was purified by column chromatography on silica gel (eluent: CH₂Cl₂/3.5M NH₃ in MeOH 50:1) to afford 3-[2-(dimethylamino)ethyl]-7-(2-fluoroethyl)indol-4-ol as white solid (65 mg, 54%). MS (ESI, m/z): 251 (M+H)⁺. ¹H NMR (400 MHz, CDCl₃) δ 8.16 (br s, 1H), 6.86 (s, 1H), 6.85 (d, J=8 Hz, 1H), 6.51 (d, J=8 Hz, 1H), 4.73 (dt, J=6 Hz (t) and 47.2 Hz(d), 2H), 3.13 (dt, J=6 Hz (t) and 28 Hz (d), 2H), 2.96-2.94 (m, 2H), 2.72-2.70 (m, 2H), 2.38 (s, 6H).

### Example of Chemical Synthesis of Compound 9

### Compound 9: 3-[2-(Dimethylamino)ethyl]-7-ethylindol-4-ol

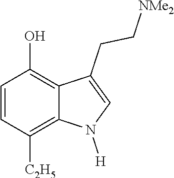 [SMILES: CCc1ccc(O)c2c(CCN(C)C)c[nH]c12]

An example of a synthesis of 3-[2-(dimethylamino)ethyl]-7-ethylindol-4-ol is provided as follows:

### [2-[4-(Benzyloxy)-7-ethylindol-3-yl]ethyl]dimethylamine

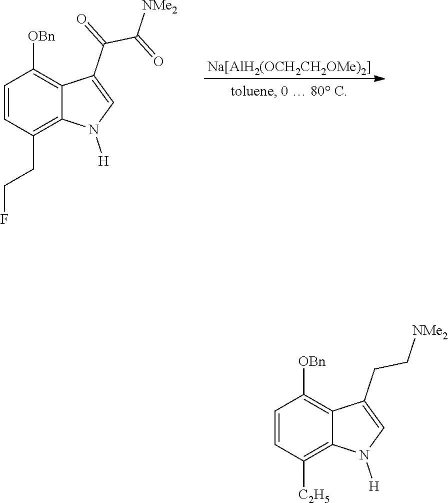

To a solution of 2-[4-(benzyloxy)-7-(2-fluoroethyl)indol-3-yl]-N,N-dimethylglyoxylamide (220 mg, 0.59 mmol) in toluene (5 mL) was added dropwise with ice cooling sodium bis(2-methoxyethoxy)aluminum dihydride solution (70% in toluene, Red-AI, 0.97 g, 4.8 mmol, 6 equiv.). The mixture was heated to 80° C. and stirred at this temperature overnight. The reaction was quenched with H₂O, and 15% aqueous NaOH solution was added. The mixture was extracted with EtOAc. The organic phase was washed with water and brine, dried over Na₂SO₄, and concentrated under reduced pressure. The residue was purified by column chromatography on silica gel (eluent: CH₂Cl₂/3.5M NH₃ in MeOH 30:1) to afford [2-[4-(benzyloxy)-7-ethylindol-3-yl]ethyl]dimethylamine as a brown solid (76 mg, 39%). MS (ESI, m/z): 323 (M+H)⁺. ¹H NMR (400 MHz, CDCl₃) δ 7.94 (br s, 1H), 7.50 (d, J=7.2 Hz, 2H), 7.38 (t, J=7.6 Hz, 2H), 7.31-7.29 (m, 1H), 6.90 (d, J=2.0 Hz, 1H), 6.87 (d, J=7.6 Hz, 1H), 6.50 (d, J=8 Hz, 1H), 5.17 (s, 2H), 3.06 (t, J=8 Hz, 2H), 2.76 (q, J=7.6 Hz, 2H), 2.63-2.59 (m, 2H), 2.15 (s, 6H), 1.32 (t, J=8 Hz, 3H).

### 3-[2-(Dimethylamino)ethyl]-7-ethylindol-4-ol

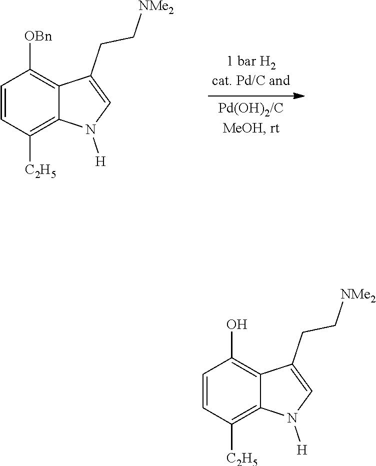 [SMILES: CCc1ccc(OCc2ccccc2)c2c(CCN(C)C)c[nH]c12 ; CCc1ccc(O)c2c(CCN(C)C)c[nH]c12]

To a solution of [2-[4-(benzyloxy)-7-ethylindol-3-yl]ethyl]dimethylamine (70 mg, 0.21 mmol) in MeOH (4 mL) was added Pd/C (20 mg) and Pd(OH)₂/C (20 mg) at hydrogen atmosphere. The resulting mixture was stirred for 2 h at 20-25° C. under a hydrogen atmosphere, then filtered. The filtrate was concentrated. The residue was purified by column chromatography on silica gel (eluent: CH₂Cl₂/3.5M NH₃ in MeOH 50:1) to afford 3-[2-(dimethylamino)ethyl]-7-ethylindol-4-ol as a white solid (30 mg, 50%). MS (ESI, m/z): 233 (M+H)⁺. ¹H NMR (400 MHz, CDCl₃) δ 7.84 (br s, 1H), 6.88 (d, J=8.0 Hz, 1H), 6.85 (d, J=2.0 Hz, 1H), 6.53 (d, J=8 Hz, 1H), 2.96-2.94 (m, 2H), 2.75 (q, J=7.6 Hz, 2H), 2.71-2.69 (m, 2H), 2.37 (s, 6H), 1.33 (t, J=8 Hz, 3H).

### Example of Chemical Synthesis of Compound 10

### Compound 10: 3-[2-(Dimethylamino)ethyl]-7-isopropylindol-4-ol

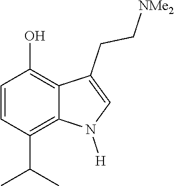 [SMILES: CC(C)c1ccc(O)c2c(CCN(C)C)c[nH]c12]

An example of a synthesis of 3-[2-(dimethylamino)ethyl]-7-isopropylindol-4-ol is provided as follows:

### 4-(Benzyloxy)-1-bromo-2-nitrobenzene

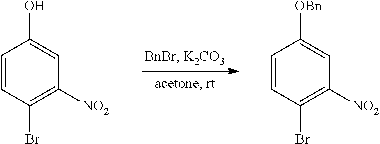 [SMILES: O=[N+]([O-])c1cc(O)ccc1Br ; O=[N+]([O-])c1cc(OCc2ccccc2)ccc1Br]

A mixture of 4-bromo-3-nitrophenol (20.0 g, 92.2 mmol), benzyl bromide (23.5 g, 138 mmol, 1.5 equiv.), K₂CO₃ (38.2 g, 277 mmol, 3.0 equiv.), and acetone (200 mL) was stirred at 20-25° C. for 16 hours, and then filtered. The filtrate was concentrated under vacuum. The residue was purified by flash chromatography on silica gel (0-20% EtOAc in petroleum ether) to yield 4-(benzyloxy)-1-bromo-2-nitrobenzene (26.8 g, 94%) as a yellow solid. ¹H NMR (300 MHz, DMSO-d₆) δ 7.76 (d, J=9.0 Hz, 1H), 7.72 (d, J=3.0 Hz, 1H), 7.48-7.32 (m, 5H), 7.26 (dd, J=9.0, 3.0 Hz, 1H), 5.18 (s, 2H).

### 4-(Benzyloxy)-7-bromoindole

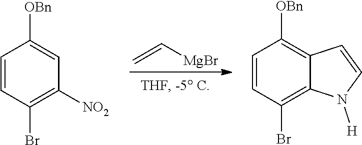

Vinylmagnesium bromide (1.0M in THF, 261 mL, 4.0 equiv.) was added dropwise to a stirred solution of 4-(benzyloxy)-1-bromo-2-nitrobenzene (20.0 g, 32.5 mmol) in THF (400 mL) at −5° C. under a N₂ atmosphere. The resulting mixture was stirred at −5° C. for 1 hour and then quenched with NH₄Cl solution and extracted three times with EtOAc. The combined organic layers were washed with water and brine, dried over Na₂SO₄, and concentrated under vacuum. The residue was purified by flash chromatography on silica gel (0-20% EtOAc in petroleum ether) to yield 4-(benzyloxy)-7-bromo-1H-indole (6.1 g, 31%) as a yellow oil. MS (ESI, m/z): 302, 304 (M+H)⁺.

### 2-[4-(Benzyloxy)-7-bromoindol-3-yl)-N,N-dimethylglyoxylamide

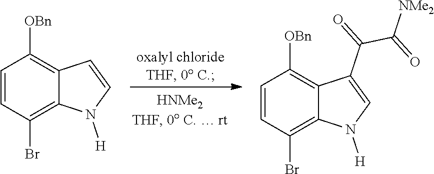 [SMILES: Brc1ccc(OCc2ccccc2)c2cc[nH]c12 ; CN(C)C(=O)C(=O)c1c[nH]c2c(Br)ccc(OCc3ccccc3)c12]

Oxalyl chloride (3.8 g, 30 mmol, 3.0 equiv.) was added dropwise to a stirred solution of 4-(benzyloxy)-7-bromoindole (3.0 g, 10.0 mmol) in THF (30 mL) at 0° C. The mixture was stirred at 20-25° C. for 16 hours. A solution of dimethylamine in THF (2.0M, 30 mL, 60 mmol, 6.0 equiv.) was added dropwise at 0° C. The resulting mixture was stirred at 20-25° C. for an additional 1 hour and then diluted with water and extracted three times with EtOAc. The combined organic layers were washed with water and brine, dried over Na₂SO₄, and concentrated under vacuum. The residue was purified by flash chromatography on silica gel (0-50% EtOAc in petroleum ether) to yield 2-[4-(benzyloxy)-7-bromoindol-3-yl]-N,N-dimethylglyoxylamide (2.0 g, 50%) as a yellow solid. MS (ESI, m/z): 401, 403 (M+H)⁺.

### 2-[4-(Benzyloxy)-7-(2-propenyl)indol-3-yl]-N,N-dimethylglyoxylamide

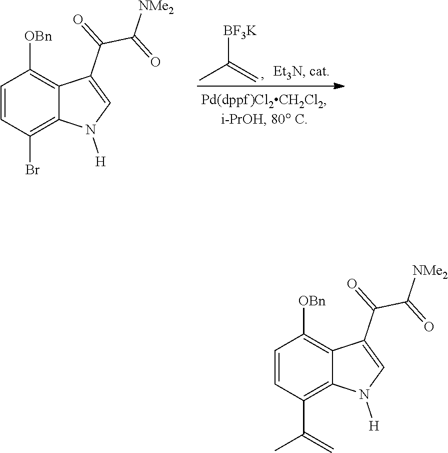

A mixture of 2-[4-(benzyloxy)-7-bromoindol-3-yl]-N,N-dimethylglyoxylamide (2.0 g, 5.0 mmol), potassium trifluoro(2-propenyl)borate (1.5 g, 10 mmol, 2.0 equiv.), Pd(dppf)Cl₂—CH₂Cl₂ (0.40 g, 0.50 mmol, 0.10 equiv.), triethylamine (1.5 g, 15 mmol, 3.0 equiv.), and isopropyl alcohol (50 mL) was stirred at 80° C. under a N₂ atmosphere for 3 hours and then filtered. The filtrate was diluted with water and extracted three times with EtOAc. The combined organic layers were washed with water and brine, dried over Na₂SO₄, and concentrated under vacuum. The residue was purified by flash chromatography on silica gel (0-80% EtOAc in petroleum ether) to yield 2-[4-(benzyloxy)-7-(2-propenyl)indol-3-yl]-N,N-dimethylglyoxylamide (1.5 g, 82%) as a brown solid. MS (ESI, m/z): 363 (M+H)⁺.

### [2-[4-(Benzyloxy)-7-(2-propenyl)indol-3-yl]ethyl]dimethylamine

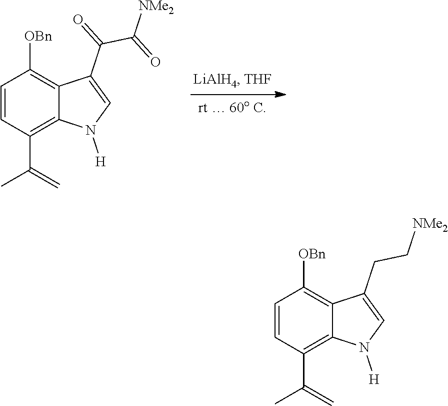 [SMILES: C=C(C)c1ccc(OCc2ccccc2)c2c(C(=O)C(=O)N(C)C)c[nH]c12 ; C=C(C)c1ccc(OCc2ccccc2)c2c(CCN(C)C)c[nH]c12]

Lithium aluminum hydride (524 mg, 13.8 mmol, 10 equiv.) was added in portions to a stirred solution of 2-[4-(benzyloxy)-7-(2-propenyl)indol-3-yl]-N,N-dimethylglyoxylamide (500 mg, 1.38 mmol) in THE (10 mL). The resulting mixture was stirred at 60° C. for 3 hours and then quenched at 0° C. with Na₂SO₄·10H₂O and filtered. The filter cake was washed three times with CH₂Cl₂. The combined filtrate and washings were concentrated under vacuum. The residue was purified by reverse phase flash chromatography on C₁₈ silica gel (5-50% acetonitrile in water containing 0.05% NH₄HCO₃) to yield [2-[4-(benzyloxy)-7-(2-propenyl)indol-3-yl]ethyl]dimethylamine (270 mg, 58%) as a brown oil. MS (ESI, m/z): 335 (M+H)⁺.

### 3-[2-(Dimethylamino)ethyl]-7-isopropylindol-4-ol

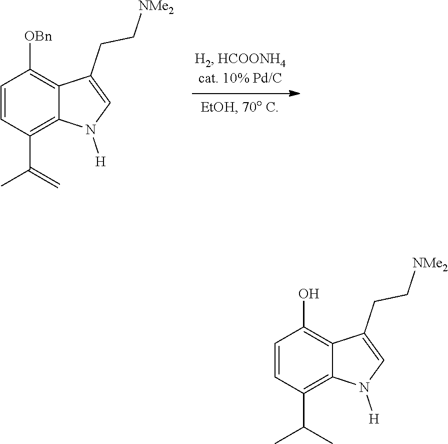 [SMILES: C=C(C)c1ccc(OCc2ccccc2)c2c(CCN(C)C)c[nH]c12 ; CC(C)c1ccc(O)c2c(CCN(C)C)c[nH]c12]

A mixture of [2-[4-(benzyloxy)-7-(2-propenyl)indol-3-yl]ethyl]dimethylamine (250 mg, 0.81 mmol), 10% Pd/C (dry, 25 mg), ammonium formate (153 mg, 2.43 mmol, 3.0 equiv.), and EtOH (8 mL) was stirred at 70° C. under a H₂ atmosphere for 3 hours and then filtered. The filtrate was concentrated under vacuum. The residue was purified by reverse phase flash chromatography on C₁₈ silica gel (5-50% acetonitrile in water containing 0.05% NH₄HCO₃) to yield 3-[2-(dimethylamino)ethyl]-7-isopropylindol-4-ol (63 mg, 34%) as a colorless solid. ¹H NMR (300 MHz, DMSO-d₆) δ 10.49 (br s, 1H), 10.34 (br s, 1H), 6.90 (d, J=2.4 Hz, 1H), 6.65 (d, J=7.8 Hz, 1H), 6.22 (d, J=7.8 Hz, 1H), 3.19 (m, 1H), 2.87 (t, J=6.6 Hz, 2H), 2.55-2.53 (m, 2H), 2.21 (s, 6H), 1.22 (d, J=6.6 Hz, 6H). MS (ESI, m/z): 247 (M+H)⁺.

### Example of Chemical Synthesis of Compound 11

### Compound 11: 3-[2-(Dimethylamino)ethyl]-7-isopropyl-1-methylindol-4-ol

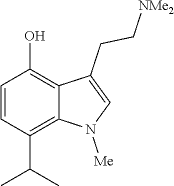 [SMILES: CC(C)c1ccc(O)c2c(CCN(C)C)cn(C)c12]

An example of a synthesis of 3-[2-(dimethylamino)ethyl]-7-isopropyl-1-methylindol-4-ol is provided as follows:

### [2-[4-(Benzyloxy)-1-methyl-7-(2-propenyl)indol-3-yl]ethyl]dimethylamine

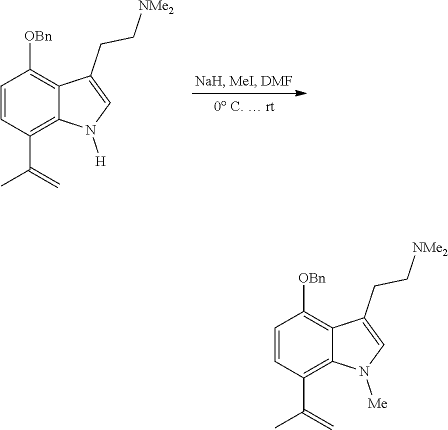 [SMILES: C=C(C)c1ccc(OCc2ccccc2)c2c(CCN(C)C)c[nH]c12 ; C=C(C)c1ccc(OCc2ccccc2)c2c(CCN(C)C)cn(C)c12]

NaH (60% in mineral oil, 216 mg, 5.4 mmol, 2.0 equiv.) was added in portions to a stirred solution of [2-[4-(benzyloxy)-7-(2-propenyl)indol-3-yl]ethyl]dimethylamine (900 mg, 2.69 mmol) in DMF (20 mL) at 0° C. The mixture was stirred at 0° C. for 10 minutes, then methyl iodide (344 mg, 2.43 mmol, 0.90 equiv.) was added. The mixture was stirred at 20-25° C. for 1 hour and then quenched with NH₄Cl solution and extracted three times with EtOAc. The combined organic layers were washed with water and brine, dried over Na₂SO₄, and concentrated under vacuum. The residue was purified by flash chromatography on silica gel (0-20% methanol in CH₂Cl₂) to yield [2-[4-(benzyloxy)-1-methyl-7-(2-propenyl)indol-3-yl]ethyl]dimethylamine (560 mg, 59%) as a yellow oil. MS (ESI, m/z): 349 (M+H)⁺.

### 3-[2-(Dimethylamino)ethyl]-7-isopropyl-1-methylindol-4-ol

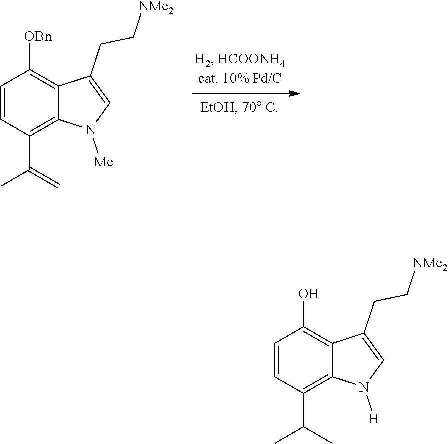 [SMILES: C=C(C)c1ccc(OCc2ccccc2)c2c(CCN(C)C)cn(C)c12 ; CC(C)c1ccc(O)c2c(CCN(C)C)c[nH]c12]

A mixture of [2-[4-(benzyloxy)-1-methyl-7-(2-propenyl)indol-3-yl]ethyl]dimethylamine (280 mg, 0.80 mmol, 1.0 equiv.), 10% Pd—C(dry, 28 mg), ammonium formate (152 mg, 2.41 mmol, 3.0 equiv.), and ethanol (10 mL) was stirred at 70° C. for 16 hours under a H₂ atmosphere and then filtered. The filtrate i was concentrated under vacuum. The residue was purified by flash chromatography on silica gel (0-30% methanol in CH₂Cl₂), and the product was isolated by trituration with EtOAc to yield 3-[2-(dimethylamino)ethyl]-7-isopropyl-1-methylindol-4-ol (11.4 mg, 5%) as an off-white solid. ¹H NMR (400 MHz, DMSO-d₆) δ 10.13 (br s, 1H), 6.79 (s, 1H), 6.75 (d, J=8.0 Hz, 1H), 6.26 (d, J=7.6 Hz, 1H), 3.87 (s, 3H), 3.66 (m, 1H), 2.85 (t, J=6.8 Hz, 2H), 2.54-2.50 (m, 2H), 2.20 (s, 6H), 1.24 (d, J=6.8 Hz, 6H). MS (ESI, m/z): 261 (M+H)⁺.

### Example of Chemical Synthesis of Compound 16

### Compound 16: 3-[2-[Isopropyl(methyl)amino]ethyl]-7-methylindol-4-ol

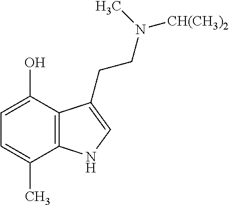 [SMILES: Cc1ccc(O)c2c(CCN(C)C(C)C)c[nH]c12]

An example of a synthesis of 3-[2-[isopropyl(methyl)amino]ethyl]-7-methylindol-4-ol is provided as follows:

### 2-[4-(Benzyloxy)-7-methylindol-3-yl]-N-isopropyl-N-methylglyoxylamide

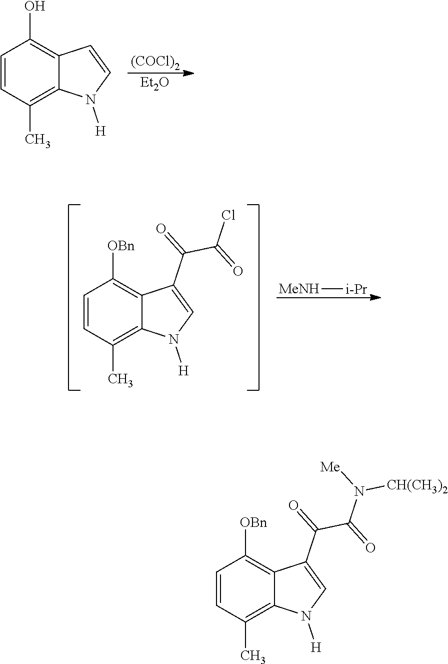

To a solution of 4-(benzyloxy)-7-methylindole (500 mg, 2.1 mmol) in dry Et₂O (10 mL) was added dropwise with ice cooling oxalyl chloride (535 mg, 4.2 mmol). The resulting brown to dark green slurry was stirred for 3 h with ice cooling. After the indole was consumed, this slurry was added dropwise with ice cooling to a solution of N-methylisopropylamine (462 mg, 6.3 mmol) and triethylamine (638 mg, 6.3 mmol) in dry Et₂O. CH₂Cl₂ (2 mL) was added. The mixture was stirred for 2 h at 0° C., warmed to room temperature, stirred overnight, and filtered. The filtrate was basified with aqueous NaHCO₃ solution, then extracted with CH₂Cl₂. The organic phase was washed with H₂O and brine, dried over Na₂SO₄, and concentrated under reduced pressure. The residue was purified by column chromatography on silica gel (eluent: CH₂Cl₂/MeOH 60:1) to afford 2-[4-(benzyloxy)-7-methylindol-3-yl]-N-isopropyl-N-methylglyoxylamide as a yellow solid (637 mg, 85%). MS (ESI, m/z): 365 (M+H)⁺.

### N-[2-[4-(Benzyloxy)-7-methylindol-3-yl]ethyl]-N-methylisopropylamine

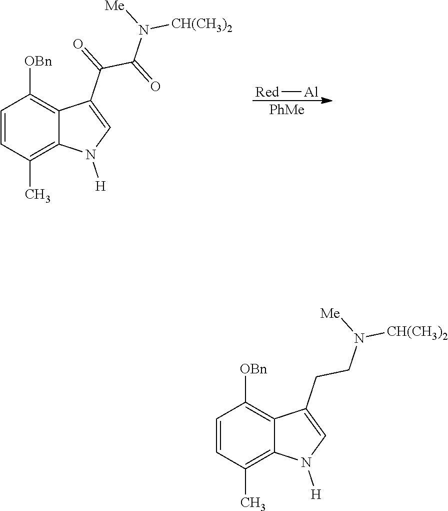

To a solution of 2-[4-(benzyloxy)-7-methylindol-3-yl]-N-isopropyl-N-methylglyoxylamide (300 mg, 0.82 mmol) in toluene (6 mL) was added dropwise with ice cooling 70% sodium bis(2-methoxyethoxy)aluminum hydride (Red-AI; 1.2 g, 4.12 mmol). The mixture was heated to 80° C. and stirred overnight. The reaction was quenched by dropwise addition of 15% aqueous NaOH solution with ice cooling. The mixture was extracted with CH₂Cl₂. The organic phase was washed with H₂O and brine, dried with Na₂SO₄, and concentrated under reduced pressure. The residue was purified by column chromatography on silica gel (CH₂Cl₂/3.5M NH₃ in MeOH 40:1) to afford N-[2-[4-(benzyloxy)-7-methylindol-3-yl]ethyl]-N-methylisopropylamine as a brown solid (251 mg, 90%). ¹H NMR (400 MHz, CDCl₃) δ 7.86 (br s, 1H), 7.48 (d, J=6.8 Hz, 2H), 7.36 (t, J=7.2 Hz, 2H), 7.30 (t, J=7.2 Hz, 1H), 6.93 (d, J=2 Hz, 1H), 6.83 (d, J=8 Hz, 1H), 6.45 (d, J=8 Hz, 1H), 5.17 (s, 2H), 3.08-3.04 (m, 2H), 2.81-2.75 (m, 1H), 2.71-2.69 (m, 2H), 2.38 (s, 3H), 2.11 (s, 3H), 0.93 (d, J=6.4 Hz, 6H).

### 3-[2-[Isopropyl(methyl)amino]ethyl]-7-methylindol-4-ol

To a solution of N-[2-[4-(benzyloxy)-7-methylindol-3-yl]ethyl]-N-methylisopropylamine (251 mg, 0.75 mmol) in MeOH (10 mL) were added 10% Pd/C (25 mg) and 20% Pd(OH)₂/C (25 mg). The reaction mixture was stirred under a hydrogen atmosphere for 3 h, then filtered, and the filtrate was concentrated under reduced pressure. The residue was purified by column chromatography on silica gel (CH₂Cl₂/3.5M NH₃ in MeOH 40:1) to afford 3-[2-[isopropyl(methyl)amino]ethyl]-7-methylindol-4-ol as a brown solid (130 mg, 71%). MS (ESI, m/z): 247 (M+H)⁺. ¹H NMR (400 MHz, CDCl₃) δ 7.77 (br s, 1H), 6.85 (d, J=2.4 Hz, 1H), 6.83 (d, J=7.6 Hz, 1H), 6.48 (d, J=7.6 Hz, 1H), 2.96-2.93 (m, 2H), 2.87 (quint, J=6.8 Hz, 1H), 2.72-2.69 (m, 2H), 2.37 (s, 3H), 2.34 (s, 3H), 0.98 (d, J=6.8 Hz, 6H).

### Example of Chemical Synthesis of Compound 18

### Compound 18: 3-[2-(Dimethylamino)ethyl]-7-methylindol-4-yl Dihydrogen Phosphate

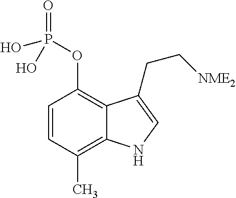

An example of a synthesis of 3-[2-(dimethylamino)ethyl]-7-methylindol-4-yl dihydrogen phosphate is provided as follows:

### Benzyl 3-[2-(Benzyldimethylammonio)ethyl]-7-methylindol-4-yl Phosphate

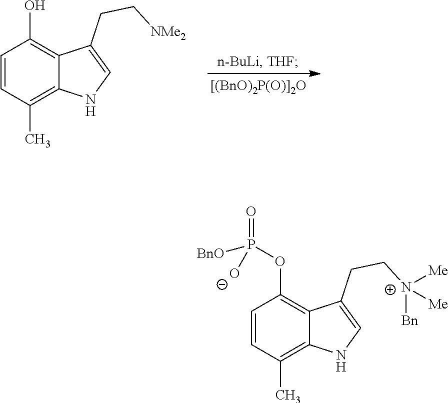 [SMILES: Cc1ccc(O)c2c(CCN(C)C)c[nH]c12 ; Cc1ccc(OP(=O)([O-])OCc2ccccc2)c2c(CC[N+](C)(C)Cc3ccccc3)c[nH]c12]

In a 1 L 3-necked flask with stir bar, Ar balloon, septum, and internal thermometer was placed 3-[2-(dimethylamino)ethyl]-7-methylindol-4-ol (9.51 g, 43.6 mmol). Addition of anhydrous THE (360 mL) produced an amber-colored solution, which was cooled in an acetone/CO₂ bath. After the internal temperature reached −70° C., a 2.5M solution of n-butyllithium in hexane (20.9 mL, 52.3 mmol, 1.2 equiv.) was added dropwise in 25 min, maintaining the internal temperature at or below −70° C. The mixture was stirred in the cold bath for 10 min, then the septum was briefly removed under a countercurrent of Ar, and tetrabenzyl pyrophosphate (25.8 g, 47.9 mmol, 1.1 equiv.) was added as a solid all at once. The flask was closed again, and the mixture was stirred for 2 h at or below −70° C. Subsequently, the temperature was allowed to rise to −30° C. over a period of 70 min, then kept in the −30 to −20° C. range for another 50 min by occasional addition of dry ice to the cold bath. Ethyl acetate (400 mL) and amino-functionalized silica gel (Supelco No. 59791; 26.5 g) were added, and the viscous mixture was kept at 20-25° C. with occasional swirling for 10 min before being filtered with suction over a layer of celite in a 600 mL fritted funnel (time-consuming because of sludgy nature of the solids). The filter residue was washed with EtOAc (4×100 mL). The combined filtrates were evaporated to obtain 23.4 g of an amber oil (containing trace amounts of a solid), which was taken up in CH₂Cl₂ (80 mL). This solution was kept with a low-intensity heat gun at its boiling point for 5 min, then was allowed to cool to 20-25° C., whereon a precipitate formed gradually. After standing overnight in a refrigerator at +5° C., the material was filtered over a 60 mL medium-porosity frit, and the fine precipitate was washed with cold CH₂Cl₂ (3×15 mL). After drying in a membrane pump vacuum (nominally 6 torr), the light-grey solid (designated fraction 1) weighed 10.5 g. Analysis by reverse-phase HPLC (column: Restek Ultra AQ C₁₈, 250×4.6 mm, 5 μm particle size. Solvent A: water with 0.05% (v/v) CF₃COOH; solvent B: CH₃CN with 0.05% (v/v) CF₃COOH. Flow: 0.8 mL/min. Gradient: 0-5 min, 50% B; 5-17 min, 50-100% B; 17-35 min, 100% B) showed a major product (t_{R} 13.9 min; 94.7 area %) and several UV-absorbing impurities, the largest of which eluted before the desired product at t_{R} 12.4 min and integrated for 4.0%. Concentration of the mother liquor to approx. 25 mL followed by storage at +5° C. gave additional precipitate, which was filtered off and was extensively washed with CH₂Cl₂ to remove most of the purple color, which had formed in the course of the handling of the mother liquor. Drying under vacuum gave another 5.2 g of a light-grey powder (designated fraction 2) with a purplish tint; total crude yield 15.7 g (75%). The HPLC purity of fraction 2 was 92.8%, with the polar, UV-absorbing impurity amounting to 5.3%.

Further purification of the above crude material could be affected in several ways. A 2.46 g sample of fraction 2 was dissolved with warming in CH₂Cl₂ (5 mL) and filtered over a 0.45 μm syringe filter followed by a 0.5 mL rinse. This solution was injected in 25 portions (each approx. 0.3 mL) onto a preparative HPLC column (same conditions as for analytical runs, but column diameter 21.2 mm and flow 17 mL/min). Each run was stopped after full elution of the product peak to conserve time and solvent. Under these conditions, the product eluted at t_{R} 12.4 min with a tail extending over several min while the major UV-absorbing impurity formed a conspicuous polar shoulder, which was rejected. The product-containing eluate was concentrated to approx. ¼ of its volume (with some CH₃CN still remaining), whereon the product initially oiled out, but subsequently began to crystallize, eventually forming star-shaped aggregates of nearly colorless needles. Swirling gradually induced remaining amorphous material to crystallize. Filtration, washing with water, and drying under vacuum gave 1.75 g of faintly purplish crystals, which were essentially pure by HPLC (above conditions). However, the ¹H and ¹³C NMR spectra (in CD₃OD) of this material displayed a conspicuous non-UV-active aliphatic/alicyclic impurity, tentatively identified as dicyclohexylurea (introduced from the tetrabenzyl pyrophosphate, as a 1H NMR spectrum of this reagent indicated). Of this partially purified material, 1.56 g was dissolved in boiling CH₃CN/water (19:1; 130 mL). On cooling to 20-25° C., crystallization set in and was completed at +5° C. overnight. Suction filtration, washing with CH₃CN, and drying under vacuum led to the recovery of 1.32 g of the product in the form of colorless crystals of 100% purity by analytical HPLC (280 nm) and lacking the putative dicyclohexylurea impurity as shown by ¹H NMR.

Alternatively, the first step of the purification sequence can be conducted by normal-phase column chromatography on silica gel. Thus, 1.70 g of fraction 2 of the crude product was dissolved in MeOH and adsorbed on silica gel (10 g). The resulting solid was placed on the top of a column of silica gel (26×4.3 cm), prepared in CHCl₃/MeOH/water/AcOH (66:29:4:1), and elution was effected with the same solvent mixture. The product spot was preceded by minor impurities and was followed by the major UV-absorbing byproduct. Analytical HPLC (280 nm) of the pooled product-containing eluate indicated a purity of 98.8 area %. The eluate was evaporated under vacuum, with the bath temperature raised to 50° C. towards the end to remove as much water and AcOH as practical, to obtain a reddish, sticky glass (2.1 g). This material was dissolved with warming in CH₃CN/water (19:1; 80 mL). Full dissolution occurred well below the boiling point, but crystallization then set in rapidly and was completed at +5° C. overnight. Isolation as above yielded 1.22 g of tan crystals of 99.8% purity by analytical HPLC (280 nm), which were lacking the putative dicyclohexylurea impurity as shown by ¹H NMR.

Recrystallization under the above conditions without prior chromatography resulted in material of lower purity than that achieved by a combination of both techniques. Thus, 0.42 g of the crude product (fraction 1) was recrystallized from CH₃CN/water (19:1; 35 mL) to recover 0.35 g of pale-grey crystals of 98.5% purity by analytical HPLC (280 nm), still retaining 1.3% of the major UV-absorbing byproduct.

Benzyl 3-[2-(benzyldimethylammonio)ethyl]-7-methylindol-4-yl phosphate: 1H NMR (CD₃OD, TMS, 500 MHz) δ 7.36-7.44 (m, 5H), 7.29-7.20 (m, 5H), 7.11 (s, 1H), 7.00, 6.79 (ABq, 2H, J=7.8 Hz, high-field part slightly broadened), 4.96 (d, 2H, J_{P-H}=5.9 Hz), 4.53 (s, 2H), 3.61, 3.45 (AA′XX′ multiplet, 4H, J_{AX}+J_{AX′}=16.9 Hz), 3.05 (s, 6H), 2.40 (s, 3H). ¹³C NMR (CD₃OD, TMS, 125 MHz) δ 145.96 (d, J_{C-P}=7.1 Hz), 139.44, 139.37 (d, J_{C-P}=8.5 Hz), 134.18 (2C), 131.75, 130.23 (2C), 129.34 (2C), 129.03, 128.76, 128.66 (2C), 124.43, 123.41, 119.80 (d, J_{C-P}=7.1 Hz), 117.58, 110.25 (d, J_{C-P}=2.4 Hz), 109.46, 69.13, 69.07 (d, J_{C-P}=5.5 Hz), 67.54, 50.29, 21.49, 16.39.

### 3-[2-(Dimethylamino)ethyl]-7-methylindol-4-yl Dihydrogen Phosphate

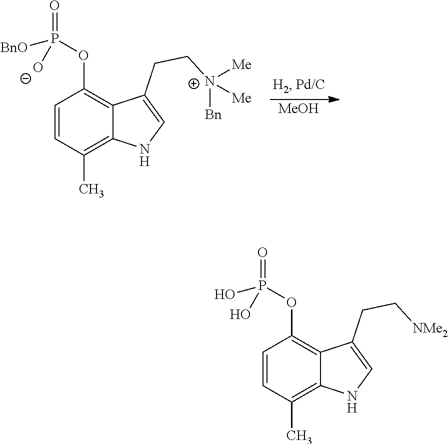 [SMILES: "MeOH" ; Cc1ccc(OP(=O)([O-])OCc2ccccc2)c2c(CC[N+](C)(C)Cc3ccccc3)c[nH]c12 ; Cc1ccc(OP(=O)(O)O)c2c(CCN(C)C)c[nH]c12]

To a solution of 2.86 g (5.98 mmol) of benzyl 3-[2-(benzyldimethylammonio)ethyl]-7-methylindol-4-yl phosphate in MeOH (140 mL) was added 10% Pd/C (wet; Oakwood No. 023236; 283 mg). The atmosphere was replaced with H₂, and the mixture was stirred under a H₂ atmosphere (balloon) at 20-25° C. for 2 h. The atmosphere was then replaced with Ar, and the suspension was filtered over a 0.45 μm PTFE membrane. Analytical HPLC (column: Restek Ultra AQ C₁₈, 250×4.6 mm, 5 μm particle size. Solvent A: water with 0.05% (v/v) CF₃COOH; solvent B: CH₃CN with 0.05% (v/v) CF₃COOH. Flow: 0.8 mL/min. Gradient: 0-5 min, 10% B; 5-25 min, 10-100% B; 25-40 min, 100% B. UV detection at 280 nm) indicated a single major product (t_{R} 13.6 min, 99.6 area %). The solution was evaporated under vacuum at a bath temperature of up to 45° C. to obtain 3-[2-(dimethylamino)ethyl]-7-methylindol-4-yl dihydrogen phosphate a brittle, off-white foam (1.73 g, 97%). This material was pure by ¹H NMR except for a small content of methanol (0.56 equiv.). ¹H NMR (CD₃OD, TMS, 500 MHz) δ 7.08 (s, 1H), 6.93, 6.81 (ABq, 2H, J=7.8 Hz, low-field part d with J=0.6 Hz), 3.40, 3.29 (AA′XX′ multiplet, 4H, J_{AX}+J_{AX′}=15.6 Hz), 2.88 (s, 6H), 2.40 (s, 3H). ¹H NMR (D₂O at lower concentration, HDO signal set to δ 4.80, 500 MHz) δ 7.23 (s, 1H), 6.98, 6.95 (ABq, 2H, J=8.0 Hz, high-field part d with J=1.1 Hz), 3.45, 3.31 (AA′XX′ multiplet, 4H, J_{AX}+J_{AX′}=14.9 Hz), 2.91 (s, 6H), 2.44 (s, 3H); ¹H NMR (D₂O at higher concentration, HDO signal set to δ 4.80, 500 MHz) δ 7.06 (s, 1H), 6.92, 6.89 (ABq, 2H, J=8.1 Hz), 3.24, 3.14 (AA′XX′ multiplet, 4H, J_{AX}+J_{AX′}=15.2 Hz), 2.79 (s, 6H), 2.36 (s, 3H). ¹³C NMR (CD₃OD, TMS, 125 MHz) δ 145.56, 139.51, 124.41, 123.29, 119.81, 118.01, 110.17 (presumably 2C coinciding), 60.44, 43.43, 23.26, 16.38. ¹³C NMR (D₂O, no standard, default calibration by software; 125 MHz) δ 143.64 (d, J_{C-P}=6.7 Hz), 137.64, 123.89, 122.27, 117.81 (d, J_{C-P}=6.7 Hz), 117.58, 108.84 (d, J_{C-P}=2.6 Hz), 108.35, 58.75, 21.46, 15.23.

### Example of Chemical Synthesis of Compound 20

### Compound 20: 3-[2-(Dimethylamino)ethyl]-7-methylindol-4-yl Acetate

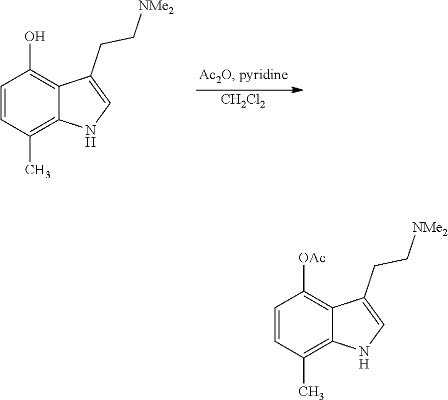 [SMILES: Cc1ccc(O)c2c(CCN(C)C)c[nH]c12 ; "CH2Cl2" ; CC(=O)Oc1ccc(C)c2[nH]cc(CCN(C)C)c12]

An example of a synthesis of 3-[2-(dimethylamino)ethyl]-7-methylindol-4-yl acetate is provided as follows:

A solution of Compound 1 (0.23 mmol) and pyridine (0.30 mmol) in CH₂Cl₂ (1 mL) is cooled with an ice bath. Acetic anhydride (255 μmol) is added. The mixture is stirred at room temperature for 2 h, or until the reaction is completed. Volatiles are removed under vacuum. The residue is purified by preparative HPLC, and the eluate evaporated, to obtain Compound 20.

### Example of Chemical Synthesis of Compound 26

### Compound 26: [2-(4-Methoxy-7-methylindol-3-yl)ethyl]dimethylamine

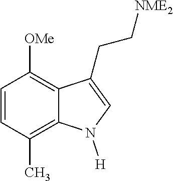

An example of a synthesis of [2-(4-methoxy-7-methylindol-3-yl)ethyl]dimethylamine is provided as follows:

### 4-Methoxy-7-methylindole

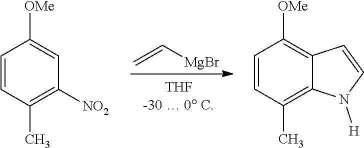

Vinylmagnesium bromide (1.0M in THF, 120 mL, 120 mmol, 4.0 equiv.) was added dropwise to a stirred solution of 4-methoxy-1-methyl-2-nitrobenzene (5.0 g, 29.9 mmol) in THE (100 mL) at −30° C. under a N₂ atmosphere. The resulting mixture was stirred at 0° C. for 2 hours and then quenched with NH₄Cl solution. The organic layer was separated and concentrated under vacuum. The residue was purified by flash chromatography on silica gel (0-30% EtOAc in petroleum ether) to yield 4-methoxy-7-methylindole (1.2 g, 24%) as a light yellow solid. MS (ESI, m/z): 162 (M+H)⁺.

### 2-[4-(Methoxy)-7-methylindol-3-yl]-N,N-dimethylglyoxylamide

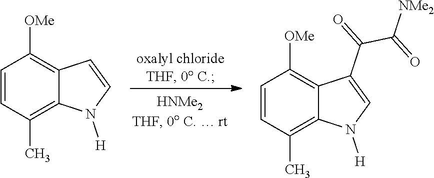 [SMILES: COc1ccc(C)c2[nH]ccc12 ; COc1ccc(C)c2[nH]cc(C(=O)C(=O)N(C)C)c12]

Oxalyl chloride (2.0 g, 9.3 mmol, 3.0 equiv.) was added dropwise to a stirred solution of 4-methoxy-7-methylindole (500 mg, 3.11 mmol) in THF (10 mL) at 0° C. under a N₂ atmosphere. The resulting mixture was stirred at 0° C. for 2 hours, and a solution of dimethylamine in THF (2.0M, 9.3 mL, 18.7 mmol, 6.0 equiv.) was added dropwise. The mixture was stirred at 20-25° C. for an additional hour and then diluted with water and extracted three times with EtOAc. The combined organic layers were washed with brine, dried over Na₂SO₄, and concentrated under vacuum. The residue was purified by flash chromatography on silica gel (0-90% EtOAc containing 0.05% triethylamine in petroleum ether containing 0.05% triethylamine) to yield 2-(4-methoxy-7-methylindol-3-yl)-N,N-dimethylglyoxylamide (500 mg, 61%) as a yellow solid. MS (ESI, m/z): 261 (M+H)⁺.

### [2-(4-Methoxy-7-methylindol-3-yl)ethyl]dimethylamine

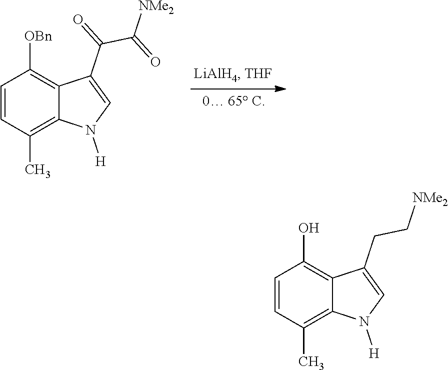 [SMILES: Cc1ccc(OCc2ccccc2)c2c(C(=O)C(=O)N(C)C)c[nH]c12 ; Cc1ccc(O)c2c(CCN(C)C)c[nH]c12]

Lithium aluminum hydride (1.46 g, 38.4 mmol, 20 equiv.) was added in portions to a stirred solution of 2-(4-methoxy-7-methylindol-3-yl)-N,N-dimethylglyoxylamide (500 mg, 1.92 mmol) in THF (20 mL) at 0° C. The resulting mixture was stirred at 65° C. for 2 hours, and then quenched at 0° C. with Na₂SO₄·10H₂O and filtered. The filtrate was concentrated under vacuum. The residue was purified by reverse phase flash chromatography on C₁₈ silica gel (0-70% acetonitrile in water) to yield [2-(4-methoxy-7-methylindol-3-yl)ethyl]dimethylamine (72 mg, 16%) as a light yellow solid. ¹H NMR (300 MHz, DMSO-d₆) δ 10.64 (br s, 1H), 6.95 (d, J=2.4 Hz, 1H), 6.72 (d, J=7.5 Hz, 1H), 6.31 (d, J=7.8 Hz, 1H), 3.78 (s, 3H), 2.94-2.82 (m, 2H), 2.47-2.39 (m, 2H), 2.32 (s, 3H), 2.19 (s, 6H). MS (ESI, m/z): 233 (M+H)⁺.

### Example of Chemical Synthesis of Compound 33

### Compound 33: 3-[2-(Dimethylamino)ethyl]-6-fluoro-7-methylindol-4-ol

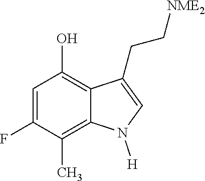

An example of a synthesis of 3-[2-(dimethylamino)ethyl]-6-fluoro-7-methylindol-4-ol is provided as follows:

### 4-Bromo-6-fluoro-7-methylindole

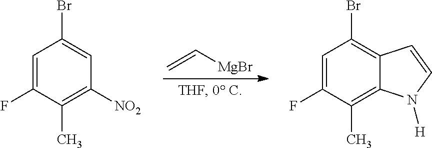

Vinylmagnesium bromide (1.0M in THF, 170 mL, 0.17 mol, 4.0 equiv.) was added dropwise to a stirred mixture of 5-bromo-1-fluoro-2-methyl-3-nitrobenzene (10.0 g, 42.7 mmol) in THF (100 mL) at 0° C. The mixture is stirred at 0° C. for 1 hour and then quenched with NH₄Cl solution. The mixture was extracted three times with EtOAc. The organic layers were combined and concentrated under vacuum. The residue was purified by flash chromatography on silica gel (0-25% EtOAc in petroleum ether) to yield 4-bromo-6-fluoro-7-methylindole (2.6 g, 24%) as a brown oil. MS (ESI, m/z): 226, 228 (M−H)⁺.

### 2-(4-Bromo-6-fluoro-7-methylindol-3-yl)-N,N-dimethylglyoxylamide

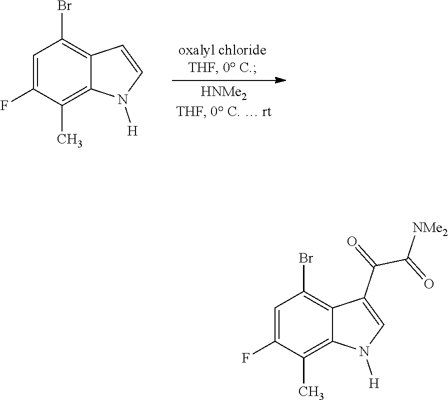 [SMILES: Cc1c(F)cc(Br)c2cc[nH]c12 ; Cc1c(F)cc(Br)c2c(C(=O)C(=O)N(C)C)c[nH]c12]

Oxalyl chloride (4.3 g, 34.2 mmol, 3.0 equiv.) was added dropwise to a stirred solution of 4-bromo-6-fluoro-7-methyl-1H-indole (2.6 g, 11.4 mmol) in THF (50 mL) at 0° C. under a N₂ atmosphere. The mixture was stirred at 0° C. for 2 h, and a solution of dimethylamine in THF (2.0M, 34 mL, 68 mmol, 6.0 equiv.) was added dropwise. The mixture was stirred at 20-25° C. for an additional hour and then quenched with NH₄Cl solution and extracted three times with EtOAc. The combined organic layers were washed with brine, dried over Na₂SO₄, and concentrated under vacuum. The residue was purified by reverse phase flash chromatography on C₁₈ silica gel (5-70% acetonitrile in water containing 0.5% NH₄HCO₃) to yield 2-(4-bromo-6-fluoro-7-methylindol-3-yl)-N,N-dimethylglyoxylamide (1.9 g, 46%) as a yellow solid. MS (ESI, m/z): 327, 329 (M+H)⁺.

### 2-(6-Fluoro-4-hydroxy-7-methylindol-3-yl)-N,N-dimethylglyoxylamide

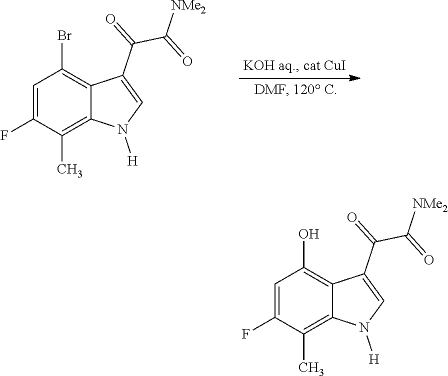 [SMILES: Cc1c(F)cc(Br)c2c(C(=O)C(=O)N(C)C)c[nH]c12 ; Cc1c(F)cc(O)c2c(C(=O)C(=O)N(C)C)c[nH]c12]

A mixture of 2-(4-bromo-6-fluoro-7-methylindol-3-yl)-N,N-dimethylglyoxylamide (4.0 g, 12.2 mmol), KOH (3.4 g as a 30% aqueous solution, 61 mmol, 5.0 equiv.), CuI (0.70 g, 3.7 mmol, 0.30 equiv.), and DMF (50 mL) was stirred at 120° C. for 1 hour under a N₂ atmosphere and then filtered. The filtrate was concentrated under vacuum. The residue was purified by reverse phase flash chromatography on C₁₈ silica gel (5-40% acetonitrile in water containing 0.5% trifluoroacetic acid) to yield 2-(6-fluoro-4-hydroxy-7-methylindol-3-yl)-N,N-dimethylglyoxylamide (0.80 g, 23%) as a yellow solid. MS (ESI, m/z): 265 (M+H)⁺.

### 3-[2-(Dimethylamino)ethyl]-6-fluoro-7-methylindol-4-ol

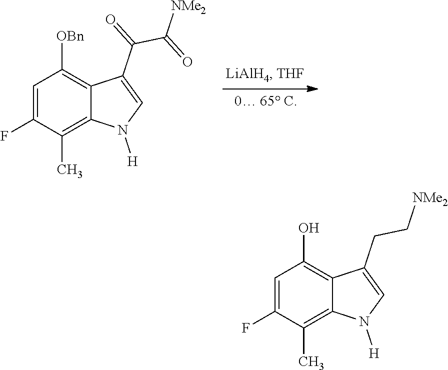 [SMILES: Cc1c(F)cc(OCc2ccccc2)c2c(C(=O)C(=O)N(C)C)c[nH]c12 ; Cc1c(F)cc(O)c2c(CCN(C)C)c[nH]c12]

A mixture of 2-(6-fluoro-4-hydroxy-7-methylindol-3-yl)-N,N-dimethylglyoxylamide (0.80 g, 3.02 mmol) and LiAlH₄ (1.1 g, 30 mmol, 10 equiv.) in THE (30 mL) was stirred at 65° C. for 16 hours. After cooling, the mixture was diluted with CH₂O2, quenched with Na₂SO₄·10H₂O, and filtered. The filtrate was concentrated under vacuum. The residue was purified by reverse phase flash chromatography on C₁₈ silica gel (5-40% acetonitrile in water containing 0.5% NH₄HCO₃) to yield 3-[2-(dimethylamino)ethyl]-6-fluoro-7-methylindol-4-ol (218 mg, 29%) as a white solid. ¹H NMR (300 MHz, DMSO-d₆): δ 11.40 (br s, 1H), 10.65 (br s, 1H), 6.93 (s, 1H), 6.10 (d, J_{H-F}=12.0 Hz, 1H), 2.87-2.83 (m, 2H), 2.55-2.51 (m, 2H), 2.22 (s, 6H), 2.19-2.18 (narrow m, 3H). ¹⁹F NMR (282 MHz, DMSO-d₆): δ −128.0. MS (ESI, m/z): 237 (M+H)⁺.

### Example of Chemical Synthesis of Compound 34

### Compound 34: [2-(6-Fluoro-4-methoxy-7-methylindol-3-yl)ethyl]dimethylamine

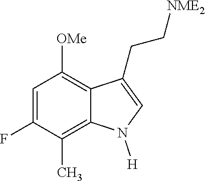

An example of a synthesis of [2-(6-fluoro-4-methoxy-7-methylindol-3-yl)ethyl]dimethylamine is provided as follows:

### 2-(6-Fluoro-4-methoxy-7-methylindol-3-yl)-N,N-dimethylglyoxylamide

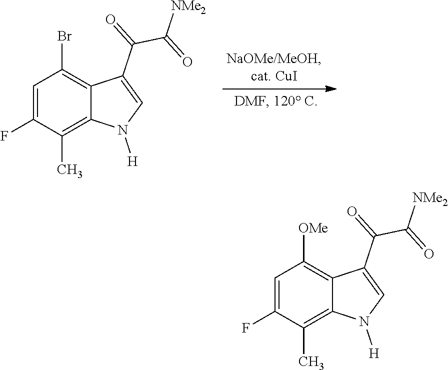 [SMILES: Cc1c(F)cc(Br)c2c(C(=O)C(=O)N(C)C)c[nH]c12 ; COc1cc(F)c(C)c2[nH]cc(C(=O)C(=O)N(C)C)c12]

A mixture of 2-(4-bromo-6-fluoro-7-methylindol-3-yl)-N,N-dimethylglyoxylamide (1.90 g, 5.80 mmol), sodium methoxide (30% solution in methanol, 3.1 g, 17.4 mmol, 3.0 equiv.), CuI (220 mg, 1.16 mmol, 0.20 equiv.), and DMF (20 mL) was stirred at 120° C. for 1 h under a N₂ atmosphere and then filtered. The filtrate was concentrated under vacuum. The residue was purified by flash chromatography on silica gel (0-10% methanol in CH₂Cl₂) to yield 2-(6-fluoro-4-methoxy-7-methylindol-3-yl)-N,N-dimethylglyoxylamide (0.80 g, 43%) as a yellow solid. MS (ESI, m/z): 279 (M+H)⁺.

### [2-(6-Fluoro-4-methoxy-7-methylindol-3-yl)ethyl]dimethylamine

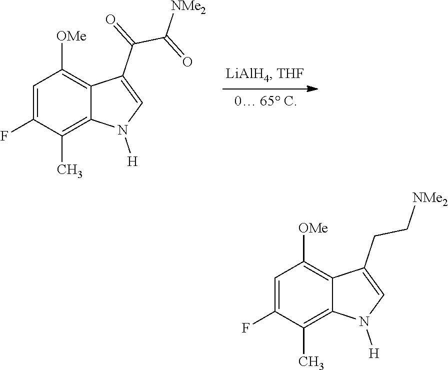 [SMILES: COc1cc(F)c(C)c2[nH]cc(C(=O)C(=O)N(C)C)c12 ; COc1cc(F)c(C)c2[nH]cc(CCN(C)C)c12]

A mixture of 2-(6-fluoro-4-methoxy-7-methylindol-3-yl)-N,N-dimethylglyoxylamide (0.80 g, 2.87 mmol), LiAlH₄ (1.1 g, 29 mmol, 10 equiv.), and THE (30 mL) was stirred at 65° C. for 16 hours. After cooling, the mixture was diluted with CH₂Cl₂, quenched with Na₂SO₄·10H₂O at 20-25° C., and filtered. The filtrate was concentrated under vacuum. The residue was purified by reverse phase flash chromatography on C₁₈ silica gel (5-40% acetonitrile in water containing 0.5% NH₄HCO₃) to yield [2-(6-fluoro-4-methoxy-7-methylindol-3-yl)ethyl]dimethylamine (55 mg, 7%) as a colorless solid. ¹H NMR (400 MHz, DMSO-d₆): δ 10.82 (brs, 1H), 6.96 (s, 1H), 6.33 (d, J=12.4 Hz, 1H), 3.80 (s, 3H), 2.87-2.83 (m, 2H), 2.47-2.43 (m, 2H), 2.24-2.23 (m, 3H), 2.19 (s, 6H). MS (ESI, m/z): 251 (M+H)⁺.

### Example of Chemical Synthesis of Compound 36

### Compound 36: 3-[2-(Dimethylamino)ethyl]-6-fluoroindol-4-ol

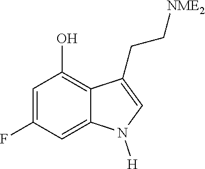

An example of a synthesis of 3-[2-(dimethylamino)ethyl]-6-fluoroindol-4-ol is provided as follows:

### 2-(Benzyloxy)-4-fluorobenzaldehyde

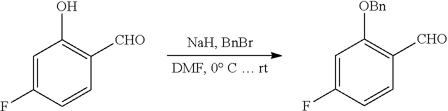 [SMILES: O=Cc1ccc(F)cc1O ; O=Cc1ccc(F)cc1OCc1ccccc1]

NaH (60% in mineral oil, 12.9 g, 536 mmol, 5 equiv.) was added in portions to a stirred solution of 4-fluoro-2-hydroxybenzaldehyde (15.0 g, 107 mmol) in DMF (150 mL) at 0° C. The mixture was stirred at 0° C. for 10 minutes. Benzyl bromide (36.4 g, 214 mmol, 2.0 equiv.) was added to the mixture. The mixture was stirred at 20-25° C. for 16 h and then quenched with NH₄Cl solution and extracted three times with EtOAc. The combined organic layers were washed with water and brine, dried over Na₂SO₄, and concentrated under vacuum. The residue was purified by flash chromatography on silica gel (0-75% EtOAc in petroleum ether) to afford 2-(benzyloxy)-4-fluorobenzaldehyde (22.0 g, 89%) as a yellow oil. MS (ESI, m/z): 231 (M+H)⁺.

### Ethyl (2Z)-2-Azido-2′-(benzyloxy)-4′-fluorocinnamate

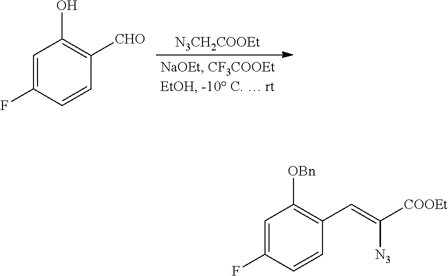 [SMILES: "N3CH2COOEt" ; O=Cc1ccc(F)cc1O ; CCOC(=O)/C(=C/c1ccc(F)cc1OCc1ccccc1)N=[N+]=[N-]]

Na metal (6.0 g, 261 mmol, 4.0 equiv.) was added with stirring and in portions to anhydrous ethanol (300 mL) at −10° C. under a N₂ atmosphere. The mixture was stirred until the Na metal was completely dissolved. To this sodium ethoxide solution, a solution of 2-(benzyloxy)-4-fluorobenzaldehyde (15.0 g, 65.2 mmol) and ethyl 2-azidoacetate (33.7 g, 261 mmol, 4.0 equiv.) in ethanol (150 mL) was added dropwise at −10° C. over a 10 minute period. The mixture was stirred for another 10 minutes, and ethyl trifluoroacetate (18.5 g, 130 mmol, 2.0 equiv.) was then added. The resulting mixture was stirred at 20-25° C. for 16 hours, quenched with NH₄Cl solution, and extracted three times with EtOAc. The combined organic layers were washed with brine, dried over Na₂SO₄, and concentrated under vacuum. The residue was purified by flash chromatography on silica gel (0-70% EtOAc in petroleum ether) to afford ethyl (2Z)-2-azido-2′-(benzyloxy)-4′-fluorocinnamate (11.0 g, 49%) as a yellow solid. MS (ESI, m/z): 341 (M+H)⁺.

### Ethyl 4-(Benzyloxy)-6-fluoroindole-2-carboxylate

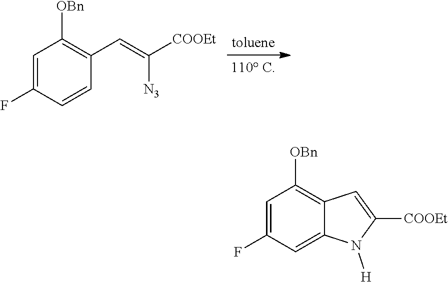 [SMILES: CCOC(=O)/C(=C/c1ccc(F)cc1OCc1ccccc1)N=[N+]=[N-] ; CCOC(=O)c1cc2c(OCc3ccccc3)cc(F)cc2[nH]1]

A solution of ethyl (2Z)-2-azido-2′-(benzyloxy)-4′-fluorocinnamate (11.0 g, 32.3 mmol) in toluene (110 mL) was stirred at 110° C. for 16 hours and then concentrated under vacuum. The residue was purified by flash chromatography on silica gel (0-85% EtOAc in petroleum ether) to yield ethyl 4-(benzyloxy)-6-fluoroindole-2-carboxylate (7.0 g, 69%) as a yellow solid. MS (ESI, m/z): 314 (M+H)⁺.

### 4-(Benzyloxy)-6-fluoroindole-2-carboxylic acid

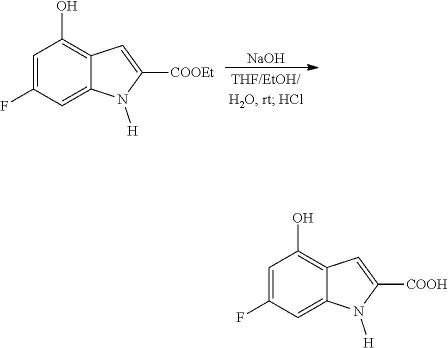 [SMILES: CCOC(=O)c1cc2c(O)cc(F)cc2[nH]1 ; "NaOH" ; O=C(O)c1cc2c(O)cc(F)cc2[nH]1]

A solution of sodium hydroxide (3.83 g, 96 mmol, 5.0 equiv.) in water (18 mL) was added to a stirred solution of ethyl 4-(benzyloxy)-6-fluoroindole-2-carboxylate (6.0 g, 19.2 mmol) in THE (18 mL) and EtOH (18 mL) at 20-25° C. The mixture was stirred at 20-25° C. for 3 hours and then acidified with HCl (1N) to pH 2-3 and extracted three times with EtOAc. The combined organic layers were washed with brine, dried over Na₂SO₄, and concentrated under vacuum to yield crude 4-(benzyloxy)-6-fluoroindole-2-carboxylic acid (4.5 g) as a yellow solid, which was used in the next step directly without further purification. MS (ESI, m/z): 286 (M+H)⁺.

### 4-(Benzyloxy)-6-fluoroindole

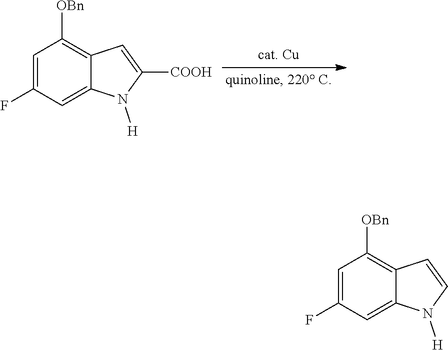 [SMILES: O=C(O)c1cc2c(OCc3ccccc3)cc(F)cc2[nH]1 ; Fc1cc(OCc2ccccc2)c2cc[nH]c2c1]

A mixture of 4-(benzyloxy)-6-fluoroindole-2-carboxylic acid (4.5 g, 15.8 mmol) and copper powder (0.30 g, 4.7 mmol, 0.30 equiv.) in quinoline (45 mL) was stirred at 220° C. for 3 days under a N₂ atmosphere and then filtered. The filter cake was washed with EtOAc. The combined filtrates were washed with HCl (1N) and brine, dried over Na₂SO₄, and then concentrated under vacuum. The residue was purified by flash chromatography on silica gel (0-25% methanol in CH₂Cl₂) to yield 4-(benzyloxy)-6-fluoroindole (500 mg, 13%) as a brown solid. MS (ESI, m/z): 242 (M+H)⁺.

### 2-[4-(Benzyloxy)-6-fluoroindol-3-yl]-N,N-dimethylglyoxylamide

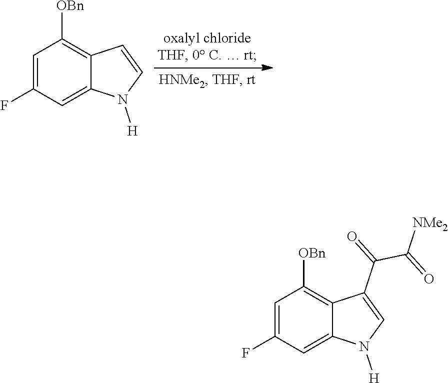 [SMILES: Fc1cc(OCc2ccccc2)c2cc[nH]c2c1 ; CN(C)C(=O)C(=O)c1c[nH]c2cc(F)cc(OCc3ccccc3)c12]

Oxalyl chloride (0.79 g, 6.2 mmol, 3.0 equiv.) was added dropwise to a stirred solution of 4-(benzyloxy)-6-fluoroindole (500 mg, 2.07 mmol) in THF (5 mL) at 0° C. The resulting mixture was stirred at 20-25° C. for 4 hours. A solution of dimethylamine in THF (2.0M, 6.2 mL, 12.4 mmol, 6.0 equiv.) was added. The mixture was stirred at 20-25° C. for 1 hour and then diluted with water and extracted three times with EtOAc. The combined organic layers were washed with water and brine, dried over Na₂SO₄, and concentrated under vacuum. The residue was purified by flash chromatography on silica gel (0-10% methanol in CH₂Cl₂) to afford 2-[4-(benzyloxy)-6-fluoroindol-3-yl]-N,N-dimethylglyoxylamide (620 mg, 85%) as a yellow solid. MS (ESI, m/z): 341 (M+H)⁺.

### [2-[4-(Benzyloxy)-6-fluoroindol-3-yl]ethyl]dimethylamine

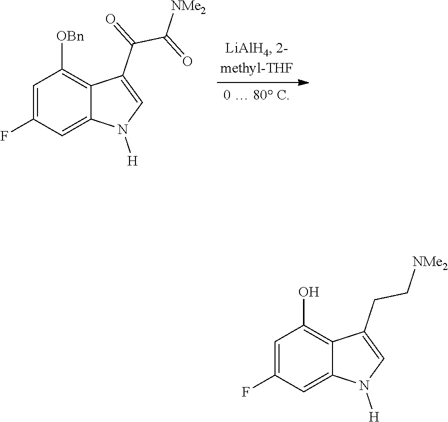 [SMILES: CN(C)C(=O)C(=O)c1c[nH]c2cc(F)cc(OCc3ccccc3)c12 ; CN(C)CCc1c[nH]c2cc(F)cc(O)c12]

Lithium aluminum hydride (1.1 g, 29 mmol, 20 equiv.) was added in portions to a stirred solution of 2-[4-(benzyloxy)-6-fluoroindol-3-yl]-N,N-dimethylglyoxylamide (500 mg, 1.47 mmol) in 2-methyltetrahydrofuran (5 mL) at 0° C. The resulting solution was stirred at 80° C. for 16 hours under a N₂ atmosphere and then quenched with Na₂SO₄·10H₂O and filtered. The filter cake was washed three times with CH₂Cl₂. The combined filtrate and washings were concentrated under vacuum to yield [2-[4-(benzyloxy)-6-fluoroindol-3-yl]ethyl]dimethylamine (500 mg crude) as a brown oil, which was used in the next step directly without further purification. MS (ESI, m/z): 313 (M+H)⁺.

### 3-[2-(Dimethylamino)ethyl]-6-fluoroindol-4-ol

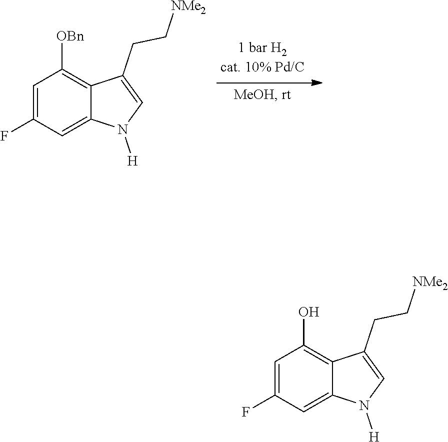 [SMILES: CN(C)CCc1c[nH]c2cc(F)cc(OCc3ccccc3)c12 ; CN(C)CCc1c[nH]c2cc(F)cc(O)c12]

A mixture of [2-[4-(benzyloxy)-6-fluoroindol-3-yl]ethyl]dimethylamine (450 mg, 1.44 mmol), 10% Pd/C (wet, 135 mg), and methanol (4.5 mL) was stirred at 20-25° C. for 3 hours under a H₂ atmosphere and then filtered. The filtrate was concentrated under vacuum. The residue was purified by reverse phase flash chromatography on C₁₈ silica gel (5-40% acetonitrile in water containing 0.05% NH₄HCO₃) to yield 3-[2-(dimethylamino)ethyl]-6-fluoroindol-4-ol (36 mg, 11%) as a colorless solid. ¹H NMR (400 MHz, DMSO-d₆) δ 10.66 (br s, 1H), 6.90 (d, J=2.0 Hz, 1H), 6.49 (dd, J=9.6, 2.4 Hz, 1H), 6.09 (dd, J=12.0, 2.4 Hz, 1H), 2.84 (t, J=6.4 Hz, 2H), 2.55 (t, J=6.4 Hz, 2H), 2.23 (s, 6H). ¹⁹F NMR (376 MHz, DMSO-d₆) δ −121.4. MS (ESI, m/z): 223 (M+H)⁺.

### Example of Chemical Synthesis of Compound 37

### Compound 37: 3-[2-(Dimethylamino)ethyl]-6-methylindol-4-ol

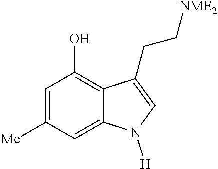

An example of a synthesis of 3-[2-(dimethylamino)ethyl]-6-methylindol-4-ol is provided as follows:

### 2-(Benzyloxy)-4-methylbenzaldehyde

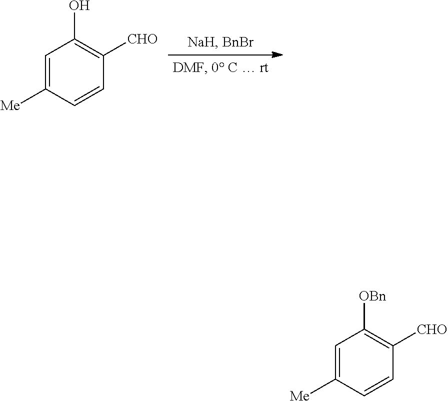 [SMILES: Cc1ccc(C=O)c(O)c1 ; Cc1ccc(C=O)c(OCc2ccccc2)c1]

NaH (60% in mineral oil, 3.5 g, 88 mmol, 1.2 equiv.) was added in portions to a stirred solution of 2-hydroxy-4-methylbenzaldehyde (10.0 g, 73.5 mmol) in DMF (100 mL) at 0° C. The mixture was stirred at 0° C. for 10 minutes, and benzyl bromide (13.8 g, 81 mmol, 1.1 equiv.) was then added. The mixture was stirred at 20-25° C. for 1 hour and then quenched with NH₄Cl solution and extracted three times with EtOAc. The combined organic layers were washed with water and brine, dried over Na₂SO₄, and concentrated under vacuum. The residue was purified by flash chromatography on silica gel (0-20% EtOAc in petroleum ether) to yield 2-(benzyloxy)-4-methylbenzaldehyde (12.2 g, 73%) as a light yellow solid. MS (ESI, m/z): 227 (M+H)⁺.

### Ethyl (2Z)-2-Azido-2′-(benzyloxy)-4′-methylcinnamate

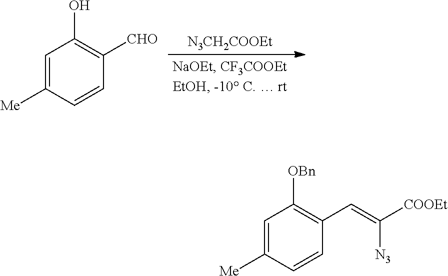 [SMILES: "N3CH2COOEt" ; Cc1ccc(C=O)c(O)c1 ; CCOC(=O)/C(=C/c1ccc(C)cc1OCc1ccccc1)N=[N+]=[N-]]

Na metal (4.1 g, 177 mmol, 4.0 equiv.) was added with stirring and in portions to anhdrous ethanol (200 mL) at −10° C. under a N₂ atmosphere. The mixture was stirred at −10° C. for 4 hours. To this sodium ethoxide solution was added a mixture of 2-(benzyloxy)-4-methylbenzaldehyde (10.0 g, 44.3 mmol), ethyl 2-azidoacetate (22.8 g, 177 mmol, 4.0 equiv.), and ethyl trifluoroacetate (12.5 g, 88 mmol, 2.0 equiv.). The reaction mixture was stirred at 20-25° C. for 2 days and then quenched with NH₄Cl solution and extracted three times with EtOAc. The combined organic layers were concentrated under vacuum. The residue was purified by flash chromatography on silica gel (0-20% EtOAc in petroleum ether) to yield ethyl (2Z)-2-azido-2′-(benzyloxy)-4′-methylcinnamate (5.7 g, 38%) as a yellow solid. MS (ESI, m/z): 338 (M+H)⁺.

### Ethyl 4-(Benzyloxy)-6-methylindole-2-carboxylate

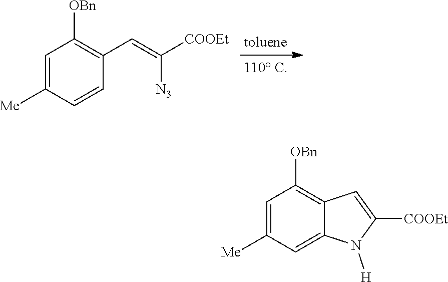 [SMILES: CCOC(=O)/C(=C/c1ccc(C)cc1OCc1ccccc1)N=[N+]=[N-] ; CCOC(=O)c1cc2c(OCc3ccccc3)cc(C)cc2[nH]1]

A solution of ethyl (2Z)-2-azido-2′-(benzyloxy)-4′-methylcinnamate (5.7 g, 16.9 mmol) in toluene (30 mL) was stirred at 100° C. for 16 hours and then concentrated under vacuum. The residue was purified by flash chromatography on silica gel (0-80% EtOAc in petroleum ether) to yield ethyl 4-(benzyloxy)-6-methylindole-2-carboxylate (5.0 g, 95%) as an off-white solid. MS (ESI, m/z): 310 (M+H)⁺.

### 4-(Benzyloxy)-6-methylindole-2-carboxylic acid

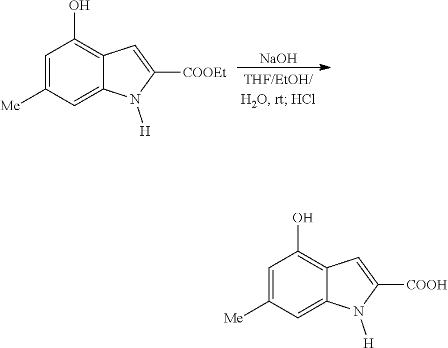 [SMILES: CCOC(=O)c1cc2c(O)cc(C)cc2[nH]1 ; "NaOH" ; Cc1cc(O)c2cc(C(=O)O)[nH]c2c1]

A solution of NaOH (1.3 g, 32 mmol, 2.0 equiv.) in water (10 mL) was added dropwise to a stirred solution of ethyl 4-(benzyloxy)-6-methylindole-2-carboxylate (5.0 g, 16.2 mmol) in THE (10 mL) and EtOH (10 mL) at 20-25° C. The resulting mixture was stirred at 20-25° C. for 16 hours and then diluted with water. The mixture was acidified with HCl (1M) to pH 1 and extracted three times with EtOAc. The combined organic layers were washed with water and brine, dried over Na₂SO₄, and concentrated under vacuum to yield crude 4-(benzyloxy)-6-methylindole-2-carboxylic acid (4.5 g, 99%) as an off-white solid, which was used for the next step directly without further purification. MS (ESI, m/z): 282 (M+H)⁺.

### 4-(Benzyloxy)-6-methylindole

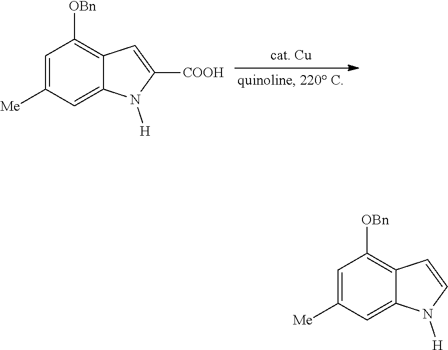 [SMILES: Cc1cc(OCc2ccccc2)c2cc(C(=O)O)[nH]c2c1 ; Cc1cc(OCc2ccccc2)c2cc[nH]c2c1]

A mixture of 4-(benzyloxy)-6-methylindole-2-carboxylic acid (2.0 g, 7.1 mmol), copper powder (0.1 g, 2 mmol, 0.3 equiv.), and quinoline (8 mL) was stirred at 220° C. for 6 days under a N₂ atmosphere. The mixture was diluted with EtOAc and then filtered. The filtrate was washed with HCl (1 M) and brine and then concentrated under vacuum. The residue was purified by reverse phase flash chromatography on C₁₈ silica gel (5-50% acetonitrile in water containing 0.05% NH₄HCO₃) to yield 4-(benzyloxy)-6-methylindole (860 mg, 50%) as a yellow solid. MS (ESI, m/z): 238 (M+H)⁺.

### 2-[4-(Benzyloxy)-6-methylindol-3-yl]-N,N-dimethylglyoxylamide

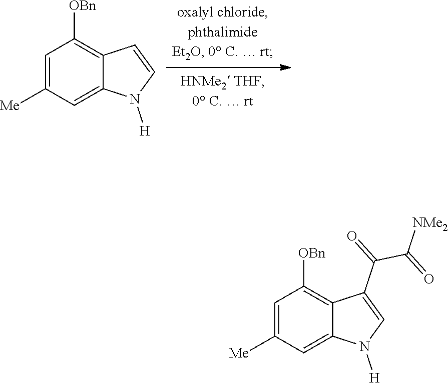 [SMILES: Cc1cc(OCc2ccccc2)c2cc[nH]c2c1 ; Cc1cc(OCc2ccccc2)c2c(C(=O)C(=O)N(C)C)c[nH]c2c1]

Oxalyl chloride (1.38 g, 10.9 mmol, 3.0 equiv.) was added dropwise to a stirred mixture of 4-(benzyloxy)-6-methylindole (860 mg, 3.63 mmol), phthalimide (347 mg, 2.36 mmol, 0.65 equiv.), and diethyl ether (110 mL) at 0° C. The mixture was stirred at 20-25° C. for 1 hour; then, a solution of dimethylamine in THF (2.0M, 10.9 mL, 21.8 mmol, 6.0 equiv.) was added dropwise at 0° C. The mixture was stirred at 20-25° C. for an additional hour and then diluted with water and extracted three times with EtOAc. The combined organic layers were washed with water and brine, dried over Na₂SO₄, and concentrated under vacuum. The residue was purified by reverse phase flash chromatography on C₁₈ silica gel (5-50% acetonitrile in water containing 0.05% NH₄HCO₃) to yield 2-[4-(benzyloxy)-6-methylindol-3-yl]-N,N-dimethylglyoxylamide (300 mg, 26%) as a yellow solid. MS (ESI, m/z): 337 (M+H)⁺.

### [2-[4-(Benzyloxy)-6-methylindol-3-yl]ethyl]dimethylamine

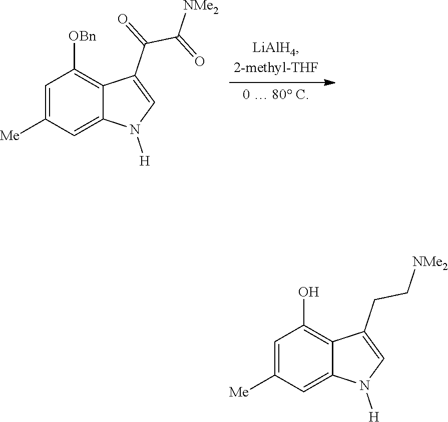 [SMILES: Cc1cc(OCc2ccccc2)c2c(C(=O)C(=O)N(C)C)c[nH]c2c1 ; Cc1cc(O)c2c(CCN(C)C)c[nH]c2c1]

Lithium aluminum hydride (0.34 g, 8.9 mmol, 10 equiv.) was added to a stirred solution of 2-[4-(benzyloxy)-6-methylindol-3-yl]-N,N-dimethylglyoxylamide (300 mg, 0.89 mmol) in 2-methyltetrahydrofuran (15 mL) at 0° C. The mixture was stirred at 80° C. for 1 hour, quenched at 0° C. with Na₂SO₄·10H₂O, and then filtered. The filtrate was concentrated under vacuum. The residue was purified by reverse phase flash chromatography on C₁₈ silica gel (5-80% acetonitrile in water containing 0.05% NH₄HCO₃) to yield [2-[4-(benzyloxy)-6-methylindol-3-yl]ethyl]dimethylamine (160 mg, 58%) as a yellow oil. MS (ESI, m/z): 309 (M+H)⁺.

### 3-[2-(Dimethylamino)ethyl]-6-methylindol-4-ol

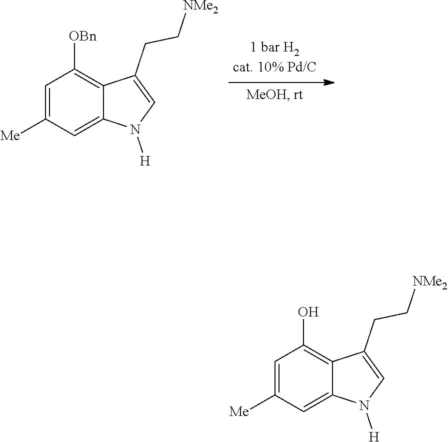 [SMILES: Cc1cc(OCc2ccccc2)c2c(CCN(C)C)c[nH]c2c1 ; Cc1cc(O)c2c(CCN(C)C)c[nH]c2c1]

A mixture of [2-[4-(benzyloxy)-6-methylindol-3-yl]ethyl]dimethylamine (160 mg, 0.52 mmol), 10% Pd—C (dry, 24 mg), and MeOH (10 mL) was stirred at 20-25° C. for 3 hours under a H₂ atmosphere and then filtered. The filtrate was concentrated under vacuum. The residue was purified by reverse phase flash chromatography on C₁₈ silica gel (5-50% acetonitrile in water containing 0.05% NH₄HCO₃) to yield 3-[2-(dimethylamino)ethyl]-6-methylindol-4-ol (23 mg, 20%) as a light yellow solid. ¹H NMR (300 MHz, DMSO-d₆) δ 10.42 (br s, 1H), 6.82 (s, 1H), 6.55 (s, 1H), 6.12 (s, 1H), 2.87-2.83 (m, 2H), 2.52-2.50 (m, 2H), 2.26-2.23 (m, 9H). MS (ESI, m/z): 219 (M+H)⁺.

### Example of Chemical Synthesis of Compound 39

### Compound 39: Dimethyl[2-[7-methyl-4-[4-(trifluoromethyl)benzyloxy]indol-3-yl]ethyl]amine

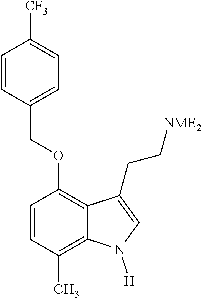

An example of a synthesis of dimethyl[2-[7-methyl-4-[4-(trifluoromethyl)benzyloxy]indol-3-yl]ethyl]amine is provided as follows:

### 2-(4-Hydroxy-7-methylindol-3-yl)-N,N-dimethylglyoxylamide

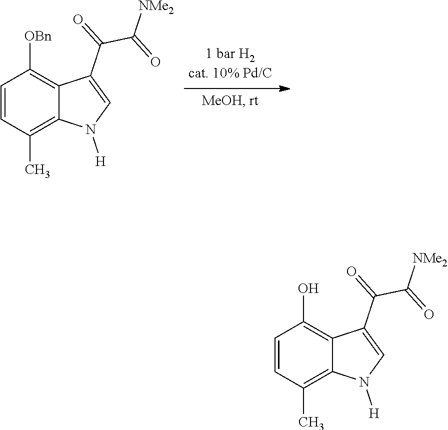 [SMILES: Cc1ccc(OCc2ccccc2)c2c(C(=O)C(=O)N(C)C)c[nH]c12 ; Cc1ccc(O)c2c(C(=O)C(=O)N(C)C)c[nH]c12]

A mixture of 2-[4-(benzyloxy)-7-methylindol-3-yl]-N,N-dimethylglyoxylamide (900 mg, 2.68 mmol), 10% Pd/C (wet, 90 mg), and MeOH (9 mL) was stirred at 20-25° C. for 2 hours under a H₂ atmosphere and then filtered. The filtrate was concentrated under vacuum to yield crude 2-(4-hydroxy-7-methylindol-3-yl)-N,N-dimethylglyoxylamide (750 mg) as a yellow solid, which was used for the next step without further purification. MS (ESI, m/z): 247 (M+H)⁺.

### N,N-Dimethyl-2-[7-methyl-4-[4-(trifluoromethyl)benzyloxy]indol-3-yl]glyoxylamide

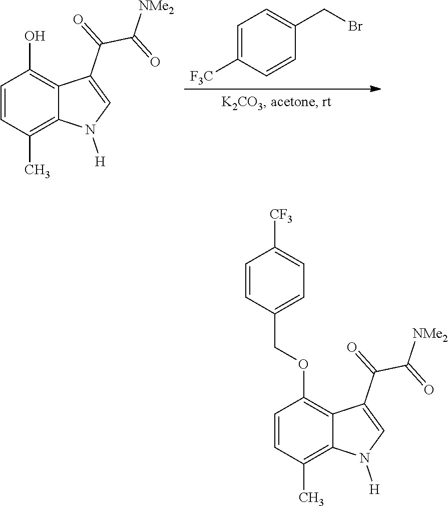 [SMILES: Cc1ccc(O)c2c(C(=O)C(=O)N(C)C)c[nH]c12 ; FC(F)(F)c1ccc(CBr)cc1 ; Cc1ccc(OCc2ccc(C(F)(F)F)cc2)c2c(C(=O)C(=O)N(C)C)c[nH]c12]

A mixture of 2-(4-hydroxy-7-methylindol-3-yl)-N,N-dimethylglyoxylamide (750 mg, 3.05 mmol), 1-(bromomethyl)-4-(trifluoromethyl)benzene (1.10 g, 4.57 mmol, 1.5 equiv.), K₂CO₃ (842 mg, 6.10 mmol, 2.0 equiv.), and acetone (4.0 mL) was stirred at 20-25° C. for 8 hours and then filtered. The filtrate was concentrated under vacuum. The residue was purified by flash chromatography on silica gel (0-50% EtOAc in petroleum ether) to yield N,N-dimethyl-2-[7-methyl-4-[4-(trifluoromethyl)benzyloxy]indol-3-yl]glyoxylamide (560 mg, 37%) as a yellow solid. MS (ESI, m/z): 405 (M+H)⁺.

### Dimethyl[2-[7-methyl-4-[4-(trifluoromethyl)benzyloxy]indol-3-yl]ethyl]amine

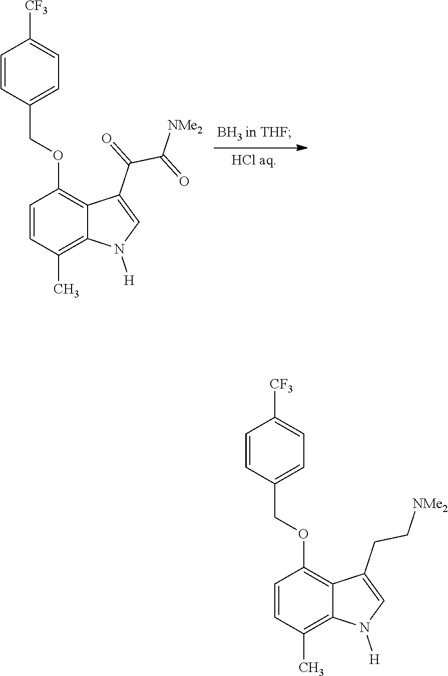 [SMILES: Cc1ccc(OCc2ccc(C(F)(F)F)cc2)c2c(C(=O)C(=O)N(C)C)c[nH]c12 ; Cc1ccc(OCc2ccc(C(F)(F)F)cc2)c2c(CCN(C)C)c[nH]c12]

BH₃·THF (1M in THF, 5.0 mL, 5.0 mmol, 5.0 equiv.) was added dropwise to a stirred solution of N,N-dimethyl-2-[7-methyl-4-[4-(trifluoromethyl)benzyloxy]indol-3-yl]glyoxylamide (400 mg, 0.99 mmol) in THF (8 mL) at 0° C. The mixture was stirred at 20-25° C. for 16 hours and then quenched with MeOH at 0° C. and extracted three times with CH₂Cl₂. The combined organic layers were washed with brine, dried over Na₂SO₄, and concentrated under vacuum. The residue was re-dissolved in THF (8 mL), and to this solution 1M aqueous HCl (4.0 mL) was added dropwise. The mixture was stirred at 50° C. for 2 days and then concentrated under vacuum. The residue was purified by preparative HPLC on C₁₈ silica gel (gradient of acetonitrile in water containing NH₄HCO₃) to afford dimethyl[2-[7-methyl-4-[4-(trifluoromethyl)benzyloxy]indol-3-yl]ethyl]amine (29 mg, 7%) as an off-white solid. ¹H NMR (400 MHz, DMSO-d₆) δ 10.23 (br s, 1H), 7.66 (d, J=8.0 Hz, 2H), 7.03-7.02 (m, 3H), 6.51 (d, J=7.6 Hz, 1H), 6.21 (d, J=7.6 Hz, 1H), 5.60 (s, 2H), 2.92 (t, J=7.2 Hz, 2H), 2.56 (t, J=7.2 Hz, 2H), 2.25 (s, 3H), 2.22 (s, 6H). MS (ESI, m/z): 377 (M+H)⁺.

### Example of Chemical Synthesis of Compound 64

### Compound 64: 3-[2-(Dimethylamino)ethyl]-4-hydroxyindole-7-carbonitrile

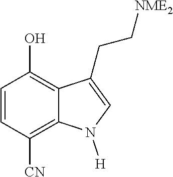

An example of a synthesis of 3-[2-(dimethylamino)ethyl]-4-hydroxyindole-7-carbonitrile is provided as follows:

### 4-(Benzyloxy)-7-bromoindole-3-carboxaldehyde

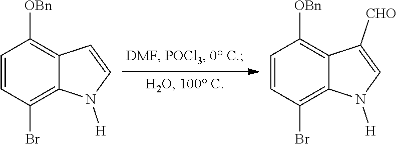 [SMILES: Brc1ccc(OCc2ccccc2)c2cc[nH]c12 ; O=Cc1c[nH]c2c(Br)ccc(OCc3ccccc3)c12]

POCl₃ (3.3 g, 22 mmol, 2.2 equiv.) was added to DMF (6.4 g, 44 mmol, 4.4 equiv.) at 0° C. The mixture was stirred at this temperature for 20 minutes. A solution of 4-(benzyloxy)-7-bromoindole (3.0 g, 10.0 mmol) in DMF (30 mL) was then added to the foregoing mixture. The resulting mixture was stirred at 0° C. for 30 minutes; water (300 mL) was then added. The resulting mixture was stirred at 100° C. for 1 hour and then quenched with aqueous NaOH solution and extracted three times with EtOAc. The combined organic layers were washed with brine and concentrated under vacuum to yield crude 4-(benzyloxy)-7-bromoindole-3-carboxaldehyde (1.6 g, 48%) as a yellow solid, which was used directly for the next step without further purification. MS (ESI, m/z): 330, 332 (M+H)⁺.

### (E)-4-(Benzyloxy)-7-bromo-3-(2-nitrovinyl)indole

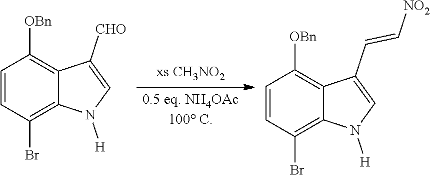 [SMILES: O=Cc1c[nH]c2c(Br)ccc(OCc3ccccc3)c12 ; O=[N+]([O-])/C=C/c1c[nH]c2c(Br)ccc(OCc3ccccc3)c12]

NH₄OAc (0.19 g, 2.4 mmol, 0.5 equiv.) was added to a stirred solution of 4-(benzyloxy)-7-bromoindole-3-carboxaldehyde (1.6 g, 4.9 mmol) in nitromethane (16 mL) at 20-25° C. under a N₂ atmosphere. The mixture was stirred at 100° C. for 2 hours and then concentrated under vacuum. The residue was purified by flash chromatography on silica gel (0-50% EtOAc in petroleum ether) to yield (E)-4-(benzyloxy)-7-bromo-3-(2-nitrovinyl)indole (1.7 g, 93%) as a yellow solid. MS (ESI, m/z): 373, 375 (M+H)⁺.

### 2-[4-(Benzyloxy)-7-bromoindol-3-yl]ethylamine

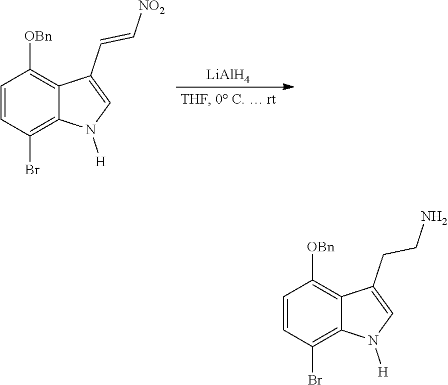 [SMILES: O=[N+]([O-])/C=C/c1c[nH]c2c(Br)ccc(OCc3ccccc3)c12 ; "LiAlH4" ; NCCc1c[nH]c2c(Br)ccc(OCc3ccccc3)c12]

Lithium aluminum hydride (0.36 g, 9.4 mmol, 5.0 equiv.) was added in portions to a stirred solution of (E)-4-(benzyloxy)-7-bromo-3-(2-nitrovinyl)indole (700 mg, 1.88 mmol) in THE (7 mL) at 0° C. and under a N₂ atmosphere. The resulting mixture was stirred at 20-25° C. for 1 hour and then quenched at 0° C. with Na₂SO₄·10H₂O and filtered. The filtrate was concentrated under vacuum to yield crude 2-[4-(benzyloxy)-7-bromoindol-3-yl]ethylamine (670 mg) as a brown solid, which was used directly for the next step without further purification. MS (ESI, m/z): 345, 347 (M+H)⁺.

### [2-[4-(Benzyloxy)-7-bromoindol-3-yl]]dimethylethylamine

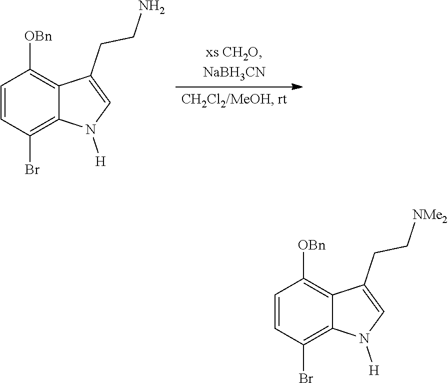 [SMILES: NCCc1c[nH]c2c(Br)ccc(OCc3ccccc3)c12 ; CN(C)CCc1c[nH]c2c(Br)ccc(OCc3ccccc3)c12]

A 30% aqueous formaldehyde solution (13.4 mL) was added to a stirred solution of 2-[4-(benzyloxy)-7-bromoindol-3-yl]ethylamine (670 mg, 1.95 mmol) in CH₂Cl₂ (8.0 mL) and MeOH (2.0 mL) at 0° C. The mixture was stirred at 20-25° C. for 10 minutes; NaBH(OAc)₃ (1.65 g, 7.8 mmol, 4.0 equiv.) was then added. The mixture was stirred at 20-25° C. for an additional 1 hour and then quenched with NaHCO₃ solution and extracted three times with EtOAc. The combined organic layers were washed three times with brine, dried over Na₂SO₄, and concentrated under vacuum. The residue was twice purified by reverse phase flash chromatography on C₁₈ silica gel (0-20% acetonitrile in water) to yield [2-[4-(benzyloxy)-7-bromoindol-3-yl]ethyl]dimethylamine (360 mg, 49%) as a brown solid. MS (ESI, m/z): 373, 375 (M+H)⁺.

### 4-(Benzyloxy)-3-[2-(dimethylamino)ethyl]indole-7-carbonitrile

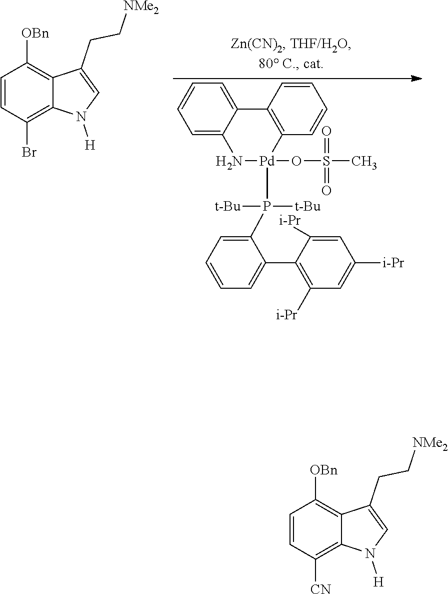

tBuXphos-Pd-G3 ([(2-di-tert-butylphosphino-2′,4′,6′-triisopropyl-1,1′-biphenyl)(2′-amino-1,1′-biphenyl-2-yl)]palladium(II) methanesulfonate, CAS registry number 1447963-75-8; 79 mg, 0.10 mmol, 0.10 equiv.) was added to a stirred solution of [2-[4-(benzyloxy)-7-bromoindol-3-yl]ethyl]dimethylamine (360 mg, 0.97 mmol) and Zn(CN)₂ (225 mg, 1.94 mmol, 2.0 equiv.) in THE (2 mL) and water (10 mL) at 20-25° C. under a N₂ atmosphere. The resulting mixture was stirred at 80° C. for 16 hours and then diluted with water and extracted three times with EtOAc. The combined organic layers were washed three times with brine, dried over Na₂SO₄, and concentrated under vacuum. The residue was purified by reverse phase flash chromatography on C₁₈ silica gel (0-60% acetonitrile in water) to yield 4-(benzyloxy)-3-[2-(dimethylamino)ethyl]indole-7-carbonitrile (170 mg, 55%) as a light yellow solid. MS (ESI, m/z): 320 (M+H)⁺.

### 3-[2-(Dimethylamino)ethyl]-4-hydroxyindole-7-carbonitrile

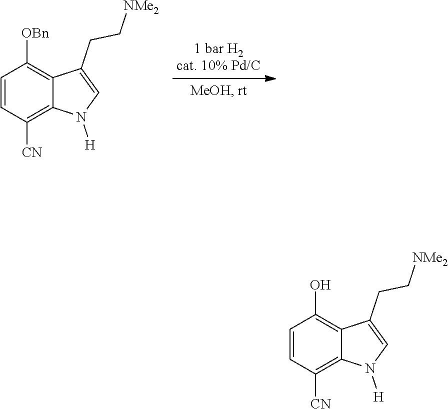 [SMILES: CN(C)CCc1c[nH]c2c(C#N)ccc(OCc3ccccc3)c12 ; CN(C)CCc1c[nH]c2c(C#N)ccc(O)c12]

A mixture of 4-(benzyloxy)-3-[2-(dimethylamino)ethyl]indole-7-carbonitrile (170 mg, 0.53 mmol) and 10% Pd/C (dry, 34 mg) in methanol (3.0 mL) was stirred at 20-25° C. for 16 hours under a H₂ atmosphere and then filtered. The filtrate was concentrated under vacuum. The residue was purified by preparative TLC on silica gel (20% methanol in CH₂Cl₂) to yield 3-[2-(dimethylamino)ethyl]-4-hydroxyindole-7-carbonitrile (48 mg, 39%) as an off-white solid. ¹H NMR (400 MHz, DMSO-d₆) δ 11.37 (br s, 1H), 7.32 (d, J=8.0 Hz, 1H), 7.05 (d, J=2.4 Hz, 1H), 6.35 (d, J=8.0 Hz, 1H), 2.91 (t, J=6.0 Hz, 2H), 2.65 (t, J=6.0 Hz, 2H), 2.30 (s, 6H). MS (ESI, m/z): 230 (M+H)⁺.

### Example of Chemical Synthesis of Compound 65

### Compound 65: 3-[2-(Dimethylamino)ethyl]-4-hydroxyindole-7-carboxamide

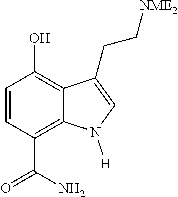

An example of a synthesis of 3-[2-(dimethylamino)ethyl]-4-hydroxyindole-7-carboxamide is provided as follows:

### 4-(Benzyloxy)-3-[2-(dimethylamino)ethyl]indole-7-carboxamide

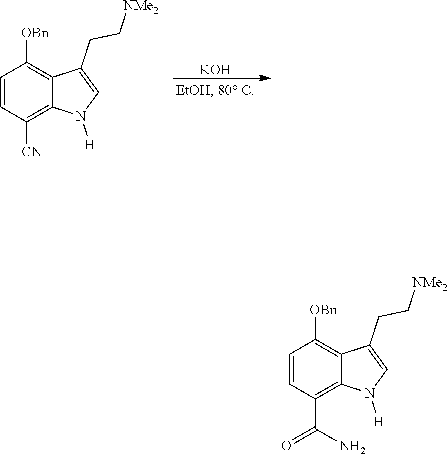 [SMILES: CN(C)CCc1c[nH]c2c(C#N)ccc(OCc3ccccc3)c12 ; "KOH" ; CN(C)CCc1c[nH]c2c(C(N)=O)ccc(OCc3ccccc3)c12]

A mixture of 4-(benzyloxy)-3-[2-(dimethylamino)ethyl]indole-7-carbonitrile (250 mg, 0.78 mmol), KOH (130 mg, 2.34 mmol, 3.0 equiv.), and EtOH (5 mL) was stirred at 80° C. for 4 hours and then filtered. The filtrate was concentrated under vacuum. The residue was purified by flash chromatography on silica gel (0-15% MeOH in CH₂Cl₂) to afford 4-(benzyloxy)-3-[2-(dimethylamino)ethyl]indole-7-carboxamide (200 mg, 69%) as a yellow solid. MS (ESI, m/z): 338 (M+H)⁺.

### 3-[2-(Dimethylamino)ethyl]-4-hydroxyindole-7-carboxamide

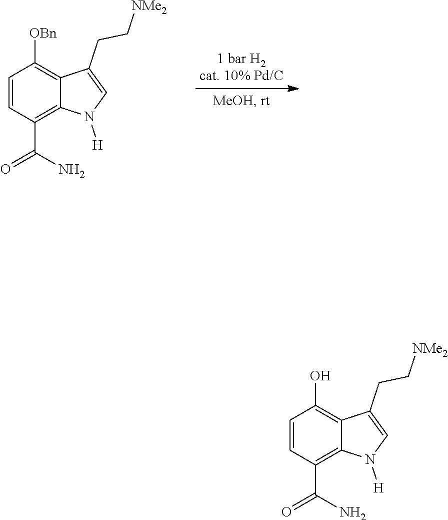 [SMILES: CN(C)CCc1c[nH]c2c(C(N)=O)ccc(OCc3ccccc3)c12 ; CN(C)CCc1c[nH]c2c(C(N)=O)ccc(O)c12]

A mixture of 4-(benzyloxy)-3-[2-(dimethylamino)ethyl]indole-7-carboxamide (190 mg, 0.56 mmol) and 10% Pd/C (wet, 57 mg) in MeOH (10 mL) was stirred at 20-25° C. for 1 hour under a H₂ atmosphere and then filtered. The filtrate was concentrated under vacuum. The residue was purified by reverse phase flash chromatography on C₁₈ silica gel (5-30% acetonitrile in water containing 0.5% NH₄HCO₃) to yield 3-[2-(dimethylamino)ethyl]-4-hydroxyindole-7-carboxamide (68 mg, 48%) as a yellow solid. ¹H NMR (300 MHz, DMSO-d₆/D₂O): δ 7.50 (d, J=8.1 Hz, 1H), 6.99 (s, 1H), 6.32 (d, J=8.1 Hz, 1H), 2.93-2.88 (m, 2H), 2.62-2.57 (m, 2H), 2.26 (s, 6H). MS (ESI, m/z): 248 (M+H)⁺.

### Example of Chemical Synthesis of Compound 77

### Compound 77: 7-Methyl-3-[((S)-1-methylazetidin-2-yl)methyl]indol-4-ol

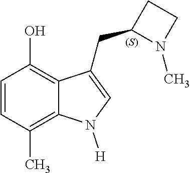

An example of a synthesis of 7-methyl-3-[((S)-1-methylazetidin-2-yl)methyl]indol-4-ol is provided as follows:

### tert-Butyl (R)-2-[4-(Benzyloxy)-7-methylindole-3-carbonyl]azetidine-1-carboxylate

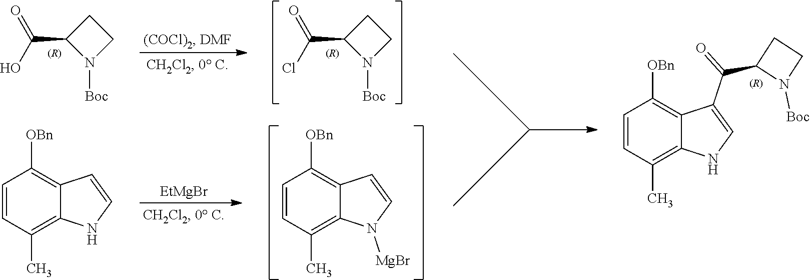

To a stirred solution of (R)-1-(tert-butoxycarbonyl)azetidine-2-carboxylic acid (916 mg, 4.55 mmol, 0.9 equiv.) in CH₂Cl₂ (10 mL) was added dropwise at 0° C. oxalyl chloride (770 mg, 6.07 mmol, 1.2 equiv.). The mixture was stirred at 0° C. for 10 min, then DMF (0.1 mL) was added. The mixture was stirred for another 1 h and then concentrated under vacuum to afford crude tert-butyl (R)-2-(chlorocarbonyl)azetidine-1-carboxylate (1.1 g) as a yellow oil.

To a stirred solution of 4-(benzyloxy)-7-methylindole (1.20 g, 5.06 mmol, 1 equiv.) in CH₂Cl₂ (12 mL) was added dropwise at 0° C. EtMgBr (2M in Et₂O, 3.0 mL, 6.0 mmol, 1.2 equiv.) under a N₂ atmosphere. The mixture was stirred at 0° C. for 0.5 h, then a solution of the above crude tert-butyl (R)-2-(chlorocarbonyl)azetidine-1-carboxylate (1.1 g) in CH₂Cl₂ (10 mL) was added. The mixture was stirred at 0° C. for another 0.5 h and then quenched with NaHCO₃ solution and extracted three times with EtOAc. The combined organic layers were washed with water and brine, dried over Na₂SO₄, and concentrated under vacuum. The residue was purified by flash chromatography on silica gel (0-80% EtOAc in petroleum ether) to afford tert-butyl (R)-2-[4-(benzyloxy)-7-methylindole-3-carbonyl]azetidine-1-carboxylate (490 mg, 23%) as a yellow solid. MS (ESI, m/z): 421 (M+H)⁺.

### 4-(Benzyloxy)-7-methyl-3-[((S)-1-methylazetidin-2-yl)methyl]indole

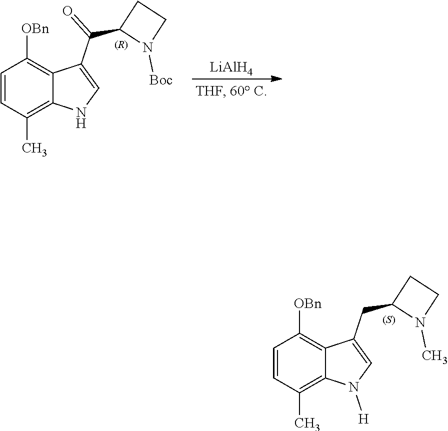

To a stirred solution of tert-butyl (R)-2-[4-(benzyloxy)-7-methylindole-3-carbonyl]azetidine-1-carboxylate (490 mg, 1.17 mmol) in THE (25 mL) at 60° C. was added in portions under N₂ atmosphere lithium aluminum hydride (442 mg, 11.7 mmol, 10.0 equiv.). The mixture was stirred at 60° C. for 16 h, cooled to 20-25° C. and quenched with Na₂SO₄·10H₂O, and filtered. The filtrate was concentrated under vacuum. The residue was purified by flash chromatography on silica gel (0-30% MeOH in CH₂Cl₂, both containing 0.05% triethylamine) to afford 4-(benzyloxy)-7-methyl-3-[((S)-1-methylazetidin-2-yl)methyl]indole (250 mg, 67%) as a yellow oil. MS (ESI, m/z): 321 (M+H)⁺.

### 7-Methyl-3-[((S)-1-methylazetidin-2-yl)methyl]indol-4-ol

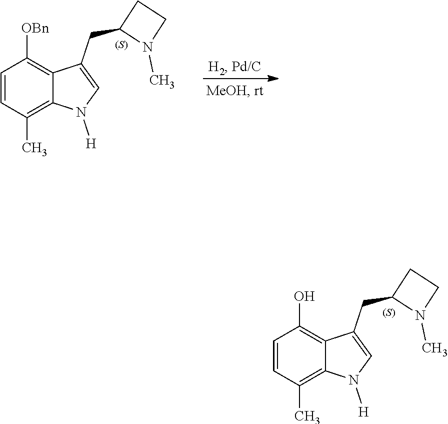

A mixture of 4-(benzyloxy)-7-methyl-3-[((S)-1-methylazetidin-2-yl)methyl]indole (250 mg, 0.78 mmol) and 10% Pd/C (wet, 75 mg) in MeOH (25 mL) was stirred at 20-25° C. for 2 h under a H₂ atmosphere. The mixture was filtered, and the filtrate was concentrated under vacuum. The residue was purified by reverse phase flash chromatography on C₁₈ silica gel (5-60% acetonitrile in water) to afford 7-methyl-3-[((S)-1-methylazetidin-2-yl)methyl]indol-4-ol (21 mg, 12%) as a grey solid. MS (ESI, m/z): 231 (M+H)⁺. ¹H NMR (300 MHz, DMSO-d₆) δ 11.76 (br s, 1H), 10.57 (br s, 1H), 6.92 (d, J=2.4 Hz, 1H), 6.60 (dd, J=7.5, 0.9 Hz, 1H), 6.19 (d, J=7.5 Hz, 1H), 3.43-3.26 (m, 2H), 3.04-2.78 (m, 3H), 2.29 (s, 3H), 2.09-2.00 (m, 4H), 1.87-1.72 (m, 1H).

### Example of Chemical Synthesis of Compound 135

### Compound 135: [2-(2,3-Dihydro-[1,4]oxazino[2,3,4-hi]indol-6-yl)ethyl]dimethylamine Hydrochloride

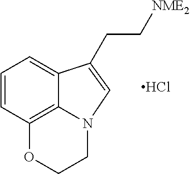

An example of a synthesis of [2-(2,3-dihydro-[1,4]oxazino[2,3,4-hi]indol-6-yl)ethyl]dimethylamine hydrochloride is provided as follows:

### 2-Hydroxyethyl 4-Toluenesulfonate

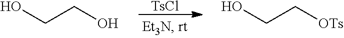

4-Toluenesulfonyl chloride (3.0 g, 17.3 mmol) was added to ethylene glycol (40 mL) at 20-25° C. After stirring for 30 min, triethylamine (2.4 mL, 17.3 mmol) was added. The mixture was stirred overnight at 20-25° C., then partitioned between EtOAc and water. The organic phase was washed with water and brine, dried over Na₂SO₄, and concentrated under reduced pressure. The residue was purified by column chromatography on silica gel (eluent: EtOAc/petroleum ether 1:3) to afford 2-hydroxyethyl 4-toluenesulfonate as a colorless liquid (3.0 g, 84%).

### 2-[7-(Benzyloxy)indol-1-yl]ethanol

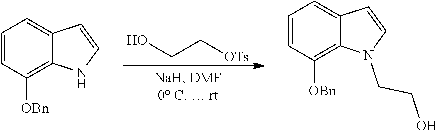 [SMILES: c1ccc(COc2cccc3cc[nH]c23)cc1 ; Cc1ccc(S(=O)(=O)OCCO)cc1 ; OCCn1ccc2cccc(OCc3ccccc3)c21]

To a solution of 7-(benzyloxy)indole (5.0 g, 22.4 mmol) in DMF (40 mL) was added with ice cooling NaH (60% dispersion in oil; 1.8 g, 45 mmol), and the resulting mixture was stirred for 30 min. A solution of 2-hydroxymethyl 4-toluenesulfonate (9.7 g, 44.8 mmol) in DMF (26 mL) was then added dropwise with ice cooling. The mixture was warmed to 20-25° C. and stirred overnight. The reaction was quenched with water, and the mixture was partitioned between water and EtOAc. The organic phase was washed with water and brine, dried over Na₂SO₄, and concentrated under reduced pressure. The residue was purified by column chromatography on silica gel (eluent: EtOAc/petroleum ether 1:6) to afford 2-[7-(benzyloxy)indol-1-yl]ethanol as a brown oil (4.3 g, 70%). ¹H NMR (400 MHz, CDCl₃) δ 7.48-7.35 (m, 5H), 7.24 (d, J=8.0 Hz, 1H), 7.03 (d, J=2.8 Hz, 1H), 7.00 (t, J=8.0 Hz, 1H), 6.72 (d, J=7.6 Hz, 1H), 6.45 (d, J=3.2 Hz, 1H), 5.17 (s, 2H), 4.46 (t, J=5.2 Hz, 2H), 3.84 (q, J=6.8 Hz, 2H), 1.44 (t, J=6.4 Hz, 1H).

### 2-[7-(Benzyloxy)indol-1-yl]ethyl Methanesulfonate

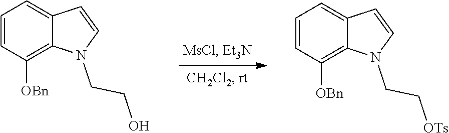 [SMILES: OCCn1ccc2cccc(OCc3ccccc3)c21 ; Cc1ccc(S(=O)(=O)OCCn2ccc3cccc(OCc4ccccc4)c32)cc1]

To a solution of 2-[7-(benzyloxy)indol-1-yl]ethanol (3.9 g, 14.6 mmol) and triethylamine (2.22 g, 21.9 mmol) in CH₂Cl₂ (75 mL) was added with ice cooling methanesulfonyl chloride (2.17 g, 19 mmol). The resulting orange solution was stirred at 20-25° C. for 0.5 h, after which time period the starting material was found to have been consumed. The reaction was quenched with H₂O, and the product was extracted into CH₂Cl₂. The organic phase was dried and concentrated under reduced pressure. The residue was purified by column chromatography on silica gel (eluent: EtOAc/petroleum ether 1:10) to afford 2-[7-(benzyloxy)indol-1-yl]ethyl methanesulfonate as a yellow oil (3.4 g, 68%).

### 2-(7-Hydroxyindol-1-yl)ethyl Methanesulfonate

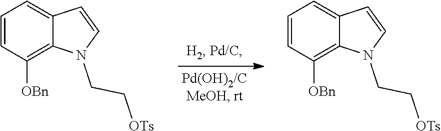 [SMILES: Cc1ccc(S(=O)(=O)OCCn2ccc3cccc(OCc4ccccc4)c32)cc1 ; Cc1ccc(S(=O)(=O)OCCn2ccc3cccc(OCc4ccccc4)c32)cc1]

To a solution of 2-[7-(benzyloxy)indol-1-yl]ethyl methanesulfonate (1.6 g, 4.6 mmol) in MeOH (45 mL) and EtOAc (10 mL) was added 10% Pd/C (250 mg) and 10% Pd(OH)₂/C (250 mg) under a nitrogen atmosphere. The reaction mixture was degassed with hydrogen and then stirred at 20-25° C. for 2 h under a hydrogen atmosphere. The catalysts were filtered off, and the filtrate was concentrated under vacuum. The residue was purified by column chromatography silica gel (eluent: EtOAc/petroleum ether 1:10) to afford 2-(7-hydroxyindol-1-yl)ethyl methanesulfonate as an off-white solid (3.0 g, 99%). ¹H NMR (400 MHz, CDCl₃) δ 7.19 (d, J=8.0 Hz, 1H), 7.04 (d, J=2.8 Hz, 1H), 6.91 (t, J=8.0 Hz, 1H), 6.52 (d, J=7.6 Hz, 1H), 6.44 (d, J=3.2 Hz, 1H), 4.70 (t, J=4.2 Hz, 2H), 4.59 (t, J=4.2 Hz, 2H), 2.39 (s, 3H).

### 2,3-Dihydro-[1,4]oxazino[2,3,4-hi]indole

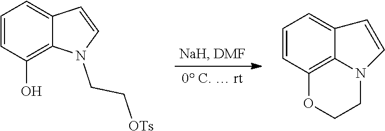 [SMILES: Cc1ccc(S(=O)(=O)OCCn2ccc3cccc(O)c32)cc1 ; c1cc2c3c(c1)ccn3CCO2]

To a solution of 2-(7-hydroxyindol-1-yl)ethyl methanesulfonate (3.1 g, 12.2 mmol) in DMF (61 mL) under ice-water bath cooling was added with ice cooling NaH (60% dispersion in oil, 0.97 g, 24.3 mmol), and the resulting mixture was stirred for 1 h, after which time period the starting material was found to have been consumed. The reaction was quenched with water, and the mixture was extracted with EtOAc. The organic phase was washed with water and brine, dried over Na₂SO₄, and concentrated under reduced pressure. The residue was purified by column chromatography on silica gel (eluent: EtOAc/petroleum ether 1:10) to afford 2,3-dihydro-[1,4]oxazino[2,3,4-hi]indole as a colorless oil (1.7 g, 88%).

### (2,3-Dihydro-[1,4]oxazino[2,3,4-hi]indol-6-yl)-N,N-dimethylglyoxylamide

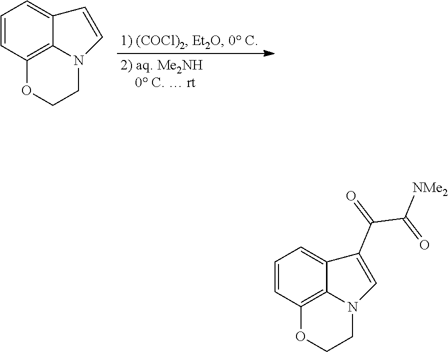 [SMILES: c1cc2c3c(c1)ccn3CCO2 ; CN(C)C(=O)C(=O)c1cn2c3c(cccc13)OCC2]

To a solution of 2,3-dihydro-[1,4]oxazino[2,3,4-hi]indole (1.70 g, 10.7 mmol) in Et₂O (50 mL) was added dropwise with ice cooling a solution of oxalyl chloride (2.7 g, 21.4 mmol) in diethyl ether (10 mL). The resulting orange slurry was stirred for 3 h with ice cooling, and was then added dropwise with ice cooling into 40% aqueous Me₂NH solution (20 mL). The resulting yellow slurry was warmed to 20-25° C. and stirred overnight. The mixture was partitioned between 10% MeOH/CH₂Cl₂ and aqueous NaHCO₃ solution. The organic phase was washed with water and brine, dried over Na₂SO₄, and concentrated under reduced pressure. The residue was purified by column chromatography on silica gel (eluent: CH₂Cl₂/3.5M NH₃ in MeOH 100:1) to afford (2,3-dihydro-[1,4]oxazino[2,3,4-hi]indol-6-yl)-N,N-dimethylglyoxylamide as a white solid (1.98 g, 72%). ¹H NMR (400 MHz, CDCl₃) δ 7.87 (s, 1H), 7.77 (d, J=8.0 Hz, 1H), 7.20 (t, J=8.0 Hz, 1H), 6.79 (d, J=7.6 Hz, 1H), 4.50 (t, J=5.2 Hz, 2H), 4.33 (t, J=5.2 Hz, 2H), 3.11 (s, 3H), 3.08 (s, 3H).

### [2-(2,3-Dihydro-[1,4]oxazino[2,3,4-hi]indol-6-yl)ethyl]dimethylamine

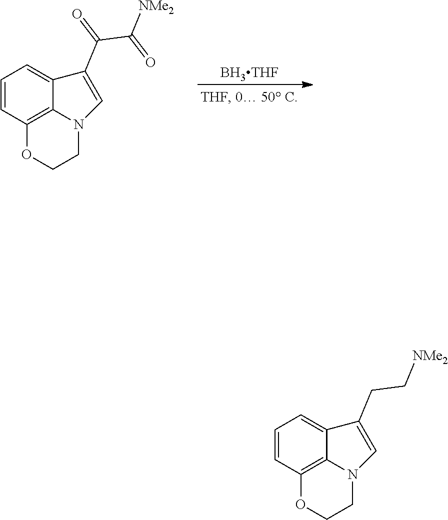 [SMILES: CN(C)C(=O)C(=O)c1cn2c3c(cccc13)OCC2 ; CN(C)CCc1cn2c3c(cccc13)OCC2 ; "BH3·THF"]

To a solution of (2,3-dihydro-[1,4]oxazino[2,3,4-hi]indol-6-yl)-N,N-dimethylglyoxylamide (200 mg, 0.78 mmol) in dry THF (7 mL) was added dropwise with ice cooling BH₃·THF solution (1.0M in THF; 3.1 mL, 4 equiv.). The mixture was heated to 50° C. and stirred overnight, then cooled to 20-25° C. MeOH was added dropwise with ice cooling. and the mixture was stirred for 2 h under reflux. The solvent was evaporated under reduced pressure, and the residue was purified by column chromatography on silica gel (eluent: CH₂Cl₂/3.5M NH₃ in MeOH 100:1) to afford [2-(2,3-dihydro-[1,4]oxazino[2,3,4-hi]indol-6-yl)ethyl]dimethylamine as a yellow oil (72 mg, 40%). MS (ESI, m/z): 231 (M+H)⁺. ¹H NMR (400 MHz, CDCl₃) δ 7.17 (d, J=8.0 Hz, 1H), 6.95 (t, J=8.0 Hz, 1H), 6.90 (s, 1H), 6.63 (d, J=7.6 Hz, 1H), 4.50 (t, J=4.4 Hz, 2H), 4.20 (t, J=4.4 Hz, 2H), 2.96-2.90 (m, 2H), 2.67-2.61 (m, 2H), 2.33 (s, 6H).

### [2-(2,3-Dihydro-[1,4]oxazino[2,3,4-hi]indol-6-yl)ethyl]dimethylamine Hydrochloride

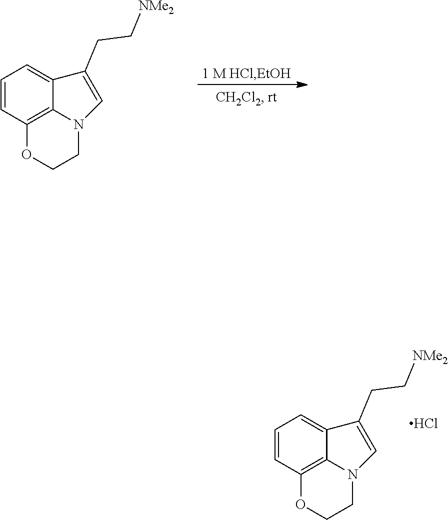

To a solution of [2-(2,3-dihydro-[1,4]oxazino[2,3,4-hi]indol-6-yl)ethyl]dimethylamine (68 mg, 0.30 mmol) in CH₂Cl₂ (1 mL) was added a solution of anhydrous HCl in EtOH (1.0M; 0.45 mL, 1.5 equiv.). The mixture was stirred at room temperature for 0.5 h. The solvent was removed under reduced pressure, and the residue was triturated with diethyl ether to afford [2-(2,3-dihydro-[1,4]oxazino[2,3,4-hi]indol-6-yl)ethyl]dimethylamine hydrochloride as a gray solid (73 mg, 93%). MS (ESI, m/z): 231 (M+H)⁺. ¹H NMR (400 MHz, CD₃OD) δ 7.20 (s, 1H), 7.19 (d, J=8.0 Hz, 1H), 6.96 (t, J=7.6 Hz, 1H), 6.59 (d, J=7.6 Hz, 1H), 4.49 (t, J=5.2 Hz, 2H), 4.27 (t, J=5.2 Hz, 2H), 3.48 (t, J=8.0 Hz, 2H), 3.23 (t, J=8.0 Hz, 2H), 2.96 (s, 6H).

### Method of Use

Indole compounds described herein are believed to be useful in the treatment of drug resistant depression based on several clinical trials that have been reported using psilocybin itself.

A US STAR*D study has reported that more than half of all patients recruited through primary care and psychiatric clinics fail to achieve remission after first-line antidepressant treatment, and one-third were unable to experience remission after four courses of acute treatment (Rush A J, Trivedi M H, Wisniewski S R, Nierenberg A A, Stewart J W, Warden D, et al. Acute and longer-term outcomes in depressed outpatients requiring one or several treatment steps: a STAR*D report. Am. J. Psychiatry 2006; 163:1905-17).

In addition to the potential use of these analogs in the treatment of depression, other studies by third party groups of human volunteers have revealed that psilocybin can be used to treat tobacco and alcohol addiction. Moreover, in a controlled clinical environment, psilocybin was safely administered to subjects with OCD, and this drug treatment was found to lead to acute reductions in core OCD symptoms in several subjects (Moreno, F. A., Wiegand, C. B., Taitano, E. K., and Delgado, P. L. “Safety, tolerability, and efficacy of psilocybin in 9 patients with obsessive-compulsive disorder” J. Clin. Psychiatry 2006, 67, 1735-1740).

Another potential use of these analogs is in the treatment of seizure disorders, including but not limited to infantile seizure disorders such as but not limited to Dravet syndrome (Sourbon, J. et al. “Serotonergic Modulation as Effective Treatment for Dravet Syndrome in a Zebrafish Mutant Model”, ACS Chem. Neurosci. 2016, 7, 588-598).

The indole compounds described herein are believed to be safer than psilocybin, given their lack of at least some of the undesirable characteristics of 5-HT_{2B}-agonist related activities.

### Methods of Administration

As contemplated herein, a therapeutically effective amount of an indole compound described herein is administered to a subject in need thereof. Whether such treatment is indicated depends on the subject case, and is further subject to medical assessment (diagnosis) that takes into consideration signs, symptoms, and/or malfunctions that are present, the risks of developing particular signs, symptoms and/or malfunctions, and other factors.

As contemplated herein, an indole compound described herein may be administered by any suitable route known in the art. Such routes include, but are not limited to, oral, buccal, inhalation, topical, sublingual, rectal, vaginal, intracisternal or intrathecal through lumbar puncture, transurethral, nasal, percutaneous, transdermal, and parenteral administration (including intravenous, intramuscular, subcutaneous, intracoronary, intradermal, intramammary, intraperitoneal, intraarticular, intrathecal, retrobulbar, intrapulmonary injection and/or surgical implantation at a particular site). Parenteral administration may be accomplished using a needle and syringe or using a high pressure technique.

Pharmaceutical compositions include those wherein an indole compound described herein is present in a sufficient amount to be administered in an effective amount to achieve its intended purpose. The exact formulation, route of administration, and dosage is determined by a qualified medical practitioner in view of the diagnosed condition or disease. Dosage amount and interval can be adjusted individually to provide levels of an indole compound described herein that is sufficient to maintain the desired therapeutic effects. It is possible that the indole compound described herein may only require infrequent administration (e.g. monthly, as opposed to daily) to achieve the desired therapeutic effect.

As contemplated herein, a therapeutically effective amount of an indole compound described herein adapted for use in therapy varies with the nature of the condition being treated, the length of time that activity is desired, and the age and the condition of the patient, and ultimately is determined by the attendant physician. Dosage amounts and intervals can be adjusted individually to provide plasma levels of the indole compound that are sufficient to maintain the desired therapeutic effects. The desired dose conveniently may be administered in a single dose, or as multiple doses administered at appropriate intervals, for example as one, two, three, four, or more subdoses per day. Multiple doses often may be desired or required. For example, an indole compound described herein may be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days' rest, and another five daily doses (5/2/5); or, any dose regimen determined to be appropriate for the circumstance.

As contemplated herein, the indole compounds described herein may be administered in admixture with a pharmaceutical carrier selected with regard to the intended route of administration and standard pharmaceutical practice. Pharmaceutical compositions for use in accordance with the indole compounds described herein are formulated in a conventional manner using one or more physiologically acceptable carriers comprising excipients and auxiliaries that facilitate processing of the compounds described herein.

Water is a preferred carrier when an indole compounds described herein is administered intravenously. Saline solutions and aqueous dextrose and glycerol solutions may also be used as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The present compositions, if desired, can also contain minor amounts of wetting or emulsifying agents, or pH buffering agents.

These pharmaceutical compositions may be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of an indole compound described herein is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of an indole compound described herein. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a compound described herein.

When a therapeutically effective amount of an indole compound described herein described herein is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains an isotonic vehicle. An indole compound described herein described herein can be infused with other fluids over a 10-30 minute span or over several hours.

The indole compounds described herein may be readily combined with pharmaceutically acceptable carriers well-known in the art. Such carriers enable the active agents to be formulated as tablets, pills, dragees, capsules, liquids, gels, syrups, slurries, suspensions and the like, for oral ingestion by a patient to be treated.

Pharmaceutical preparations for oral use can be obtained by adding an indole compound described herein to a solid excipient, with or without grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

An indole compound described herein may be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of an indole compounds described herein can be prepared as appropriate oily injection suspensions. Suitable lipophilic solvents or vehicles include fatty oils or synthetic fatty acid esters. Aqueous injection suspensions can contain substances which increase the viscosity of the suspension.

In some embodiments, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

An indole compound described herein also may be formulated in rectal compositions, such as suppositories or retention enemas, e.g., containing conventional suppository bases. In addition to the formulations described previously, an indole compound described herein also can be formulated as a depot preparation. Such long-acting formulations can be administered by implantation (for example, subcutaneously or intramuscularly) or by intramuscular injection. Thus, for example, an indole compound described herein may be formulated with suitable polymeric or hydrophobic materials (for example, as an emulsion in an acceptable oil) or ion exchange resins.

An indole compound described herein may be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The indole compounds described herein also may be injected parenterally, for example, intravenously, intramuscularly, subcutaneously, or intracoronarily. For parenteral administration, the indole compounds described herein may be best used in the form of a sterile aqueous solution which can contain other substances, for example, salts or monosaccharides, such as mannitol or glucose, to make the solution isotonic with blood. At least in some embodiments, indole compounds described herein are psilocybin analogs.

### General:

It is contemplated that any part of any aspect or embodiment discussed in this specification may be implemented or combined with any part of any other aspect or embodiment discussed in this specification. While particular embodiments have been described in the foregoing, it is to be understood that other embodiments are possible and are intended to be included herein. It will be clear to any person skilled in the art that modification of and adjustment to the foregoing embodiments, not shown, is possible.

Unless defined otherwise, all technical and scientific terms used herein have the same meaning as is commonly understood by one of ordinary skill in the art to which this invention belongs. In addition, any citation of references herein is not to be construed nor considered as an admission that such references are prior art to the present invention.

The scope of the claims should not be limited by the example embodiments set forth herein, but should be given the broadest interpretation consistent with the description as a whole.

1-20. (canceled)

21. A chemical compound of Formula I: 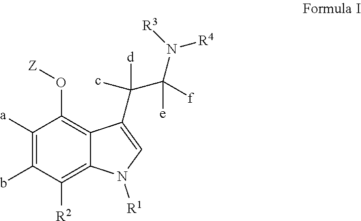
wherein:
R¹: (i) is selected from the group consisting of H and C₁-C₆ alkyl; or (ii) together with R² form a chain of 2 to 4 carbon atoms to which are attached substituents independently selected from the group consisting of H, C₁-C₆ alkyl, aryl, heteroaryl, and any combination thereof;
R²: (i) is selected from the group consisting of C₁-C₆ alkyl, aryl, heteroaryl, CN, C(O)NH₂, C(O)NH(C₁-C₆ alkyl), C(O)N(C₁-C₃ alkyl)(C₁-C₆ alkyl), C(═NOH)(C₁-C₆ alkyl), and C(═NOH)(C₁-C₆ substituted alkyl); or (ii) together with R¹ form a chain of 2 to 4 carbon atoms to which are attached substituents independently selected from the group consisting of H, C₁-C₆ alkyl, aryl, and heteroaryl; or (iii) together with b form a chain of 3 or 4 atoms, one atom of which is selected from the group consisting of C, N, and O, while the remainder are carbon, which chain contains 0, 1, or 2 double bonds, and to which chain are attached substituents independently selected from the group consisting of H, halogen, OH, C₁-C₆ alkoxy, C₁-C₆ alkyl, C₁-C₆ substituted alkyl, CF₃, and cyano; or (iv) is selected from the group consisting of H and C₁-C₆ alkyl, if b is halogen, CHF₂, CF₃, OCH₃, or OCHF₂;
a: (i) is selected from the group consisting of H and halogen; or (ii) together with Z form one of (A) a saturated chain of one oxygen and one carbon atom with oxygen connected to the 5-position of the indole ring of Formula I, and (B) a chain of 2 or 3 carbon atoms, to which chain are attached substituents independently selected from the group consisting of H and halogen, and (C) a chain of 2 or 3 carbon atoms containing one double bond;
b: (i) is selected from a group consisting of H, halogen, CH₃, CHF₂, CF₃, OCH₃, and cyano; or (ii) together with R² form a chain of 3 or 4 atoms, one atom of which is selected from the group consisting of C, N, and O, while the remainder are carbon, which chain contains 0, 1, or 2 double bonds, and to which chain are attached substituents independently selected from the group consisting of H, halogen, OH, C₁-C₆ alkoxy, C₁-C₆ alkyl, CF₃, and cyano;
R³: (i) is selected from the group consisting of H and C₁-C₆ alkyl; or (ii) together with R⁴ and the N atom to which they are attached form a 4-7 membered heterocyclyl ring; or (iii) together with f and the N atom to which R³ is attached form an azetidine or pyrrolidine ring; and
R⁴: (i) is selected from the group consisting of H and C₁-C₆ alkyl; or (ii) together with R³ and the N atom to which they are attached form a 4-7 membered heterocyclyl ring;
wherein:
c, d, e, and f are each H; or three of c, d, e, and f are H and the remaining substituent is a lower alkyl group; or c and f are each H, and d and e together are —CH₂— or —CH₂CH₂—, thereby giving rise to a cyclopropane or cyclobutane ring; or c, d, and e are each H, and f, R³, and the N atom to which R³ is attached form together an azetidine or pyrrolidine ring, such ring carrying substituents independently selected from the group consisting of H, aryl, heteroaryl, C₁-C₆ alkyl, and C₃-C₆ cycloalkyl; or d, e, and f are each H, and c, R³, and the N atom to which R³ is attached form together an azetidine or pyrrolidine ring, such ring carrying substituents independently selected from the group consisting of H, aryl, heteroaryl, halogen, C₁-C₆ alkyl, and C₃-C₆ cycloalkyl; or d, e, and f are each H, and c and Z together comprise 1 or 2 carbon atoms so as to give rise to a pyran or oxepan ring, such ring carrying substituents independently selected from the group consisting of H, halogen, C₁-C₆ alkyl, and C₃-C₆ cycloalkyl; and
wherein:
Z: (i) is selected from the group consisting of H, R⁵, (R⁶)(R⁷)N—C(O)—, C₁-C₆ alkyl-C(O), C₃-C₆ cycloalkyl-C(O), aryl-C(O), and heteroaryl-C(O), wherein R⁵ is selected from the group consisting of C₁-C₆ alkyl, and wherein R⁶ and R⁷ are each independently selected from the group consisting of H and C₁-C₄ alkyl; or (ii) is (R⁸O)(R⁹O)P(O)—, wherein R⁸ and R⁹ are each independently H or a cationic counterion of a phosphate salt form; or (iii) together with c form a linkage that gives rise to a pyran or oxepan ring; or (iv) together with a form one of (A) a saturated chain of one oxygen and one carbon atom (with oxygen connected to the 5-position of the indole ring of Formula I), and (B) a chain of 2 or 3 carbon atoms, and (C) a chain of 2 or 3 carbon atoms containing one double bond; and
any isotopologue and any pharmaceutically acceptable salt thereof.

22. The chemical compound as claimed in claim 21, wherein: (i) R¹ is selected from the group consisting of H and C₁-C₆ alkyl; and (ii) R² is C₁-C₆ alkyl, C₁-C₆ substituted alkyl, and CN;

23. The chemical compound as claimed in claim 22, wherein: (i) a is H; and (ii) b is selected from the group consisting of H and halogen;

24. The chemical compound as claimed in claim 23, wherein each of c, d, e, and f is H.

25. The chemical compound as claimed in claim 24, wherein Z is selected from the group consisting of H, C₁-C₆ alkyl, and (HO)(HO)P(O)—.

26. The chemical compound as claimed in claim 25, wherein each of R³ and R⁴ is C₁-C₆alkyl.

27. The chemical compound as claimed in claim 26, wherein: (i) R² is selected from the group consisting of methyl and ethyl; and (ii) b is selected from the group consisting of H and F.

28. The chemical compound as claimed in claim 27, wherein R¹ is H.

29. The chemical compound as claimed in claim 28, wherein each of R³ and R⁴ is methyl.

30. The chemical compound as claimed in claim 21, wherein: (i) R¹ is H; (ii) each of R², R³, and R⁴ is CH₃; and (iii) each of a, b, c, d, e, and f is H.

31. The chemical compound as claimed in claim 21, wherein: (i) R¹ is H; (ii) each of R², R³, and R⁴ is CH₃; (iii) each of a, c, d, e, and f is H; and (iv) b is F.

32. A method of treating a disorder comprising administering to a patient an effective amount of the compound as claimed in claim 30.

33. The method as claimed in claim 32, wherein the disorder is selected from the group consisting of major depressive disorder, drug resistant depression, and psychotic depression, addiction including alcoholism, tobacco addiction, cocaine addiction, and opioid addiction, pain indications including neuropathic pain, pain from chemotherapy associated neuropathy, phantom limb pain and fibromyalgia, inflammation (including chronic and acute), eating disorders including anorexia, autism, cluster headaches, migraines, dementia including Alzheimer's dementia, Parkinson's disease dementia, and Lewy body dementia, post-traumatic stress disorder, emotional distress associated with cancer, Fragile-X syndrome, autism spectrum disorder, bipolar disease, obsessive compulsive disease, and Rett syndrome.

34. A chemical compound of Formula II: 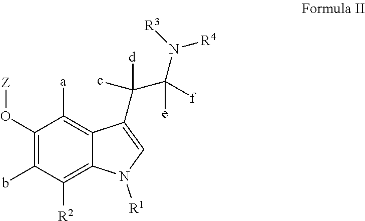
wherein:
R¹: (i) is selected from the group consisting of H, C₁-C₆ alkyl, and C₁-C₆ substituted alkyl; or (ii) together with R² form a chain of 2 to 4 carbon atoms;
R²: (i) is selected from the group consisting of C₁-C₆ alkyl; or (ii) together with R¹ form a chain of 2 to 4 carbon atoms; or (iv) is selected from the group consisting of H and C₁-C₆ alkyl, if b is halogen, CHF₂ or CF₃;
a: (i) is selected from the group consisting of H and halogen; or (ii) together with Z form one of (A) a saturated chain of one oxygen and one carbon atom, and (B) a chain of 2 or 3 carbon atoms, to which chain are attached substituents independently selected from the group consisting of H and halogen, and (C) a chain of 2 or 3 carbon atoms containing one double bond;
b is selected from a group consisting of H, halogen, CHF₂, and CF₃;
R³: (i) is selected from the group consisting of H and C₁-C₆ alkyl; or (ii) together with R⁴ and the N atom to which they are attached form a 4-7 membered heterocyclyl ring; or (iii) together with f and the N atom to which R³ is attached form an azetidine or pyrrolidine ring; or (iv) together with d form a —CH₂CH₂— linkage, or —CH₂— linkage;
R⁴: (i) is selected from the group consisting of H, C₁-C₆ alkyl, cyclopropyl, phenyl, and substituted phenyl; or (ii) together with R³ and the N atom to which they are attached form a 4-7 membered heterocyclyl ring; or (iii) together with f form a —CH₂CH₂— linkage;
c, d, e, and f are each H; or three of c, d, e, and f are H and the remaining substituent is a lower alkyl group; or c and f are each H, and d and e together are —CH₂— or —CH₂CH₂—, thereby giving rise to a cyclopropane or cyclobutane ring; or c, d, and e are each H, and f, R³, and the N atom to which R³ is attached form together an azetidine or pyrrolidine ring; or d, e, and f are each H, and c and a together are —CH₂CH₂—, —CH₂CH₂CH₂—, or —CH₂OCH₂—; and
Z: (i) is selected from the group consisting of H, phenyl, —OOCC(CH₃)₃, acetyl, —C(O)N(H)-i-Pr, —C(O)NMe₂; or (ii) is (R⁸O)(R⁹O)P(O)—, wherein R⁸ and R⁹ are each independently H or a cationic counterion of a phosphate salt form; or (iii) together with a form a —CH₂CH₂— linkage, —CH(Me)CH₂— linkage, —CH₂CH₂CH₂-linkage, or —CH═CH— linkage; and
any isotopologue and any pharmaceutically acceptable salt thereof.

35. A chemical compound of Formula III: 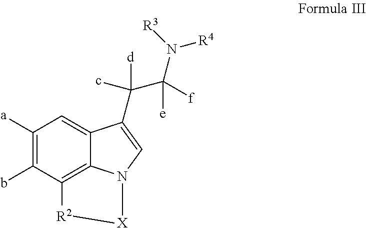
wherein:
R² and X form a —OCH₂CH₂— linkage, a —OCH₂CH₂CH₂— linkage, a —SCH₂CH₂— linkage, or a —SCH₂CH₂CH₂— linkage;
a, b, c, d, e, and f are each H; and
R³ and R⁴ are each methyl; and
any isotopologue and any pharmaceutically acceptable salt thereof.
